(19) United States
(12) Patent Application Publication
(10) Pub. No.: US 2026/0263350 A1
(43) Pub. Date: Sep. 10, 2026

(54) COMPOSITIONS

(71) Applicant: Estrenue Pty Ltd, North Adelaide, South Australia (AU)

(72) Inventors: Tarl Prow, York (GB); Stephen Nigel Birrell, Piccadilly, South Australia (AU)

(21) Appl. No.: 18/873,686

(22) PCT Filed: Jun. 9, 2023
(86) PCT No.: PCT/AU2023/050511

§ 371 Date: Dec. 10, 2024

(30) Foreign Application Priority Data
Jun. 10, 2022 (AU) 2022901602
Mar. 28, 2023 (AU) 2023900867

(51) Int. Cl. A61K 9/00; A61K 9/16; A61K 9/50; A61K 31/565

(52) U.S. Cl. CPC A61K 9/0014; A61K 9/1611; A61K 9/1617; A61K 9/1652; A61K 9/5015; A61K 31/565

(57) ABSTRACT

Disclosed herein are compositions that are formulated for topical use. The compositions may comprise microparticles, at least one bioactive compound, and optionally a penetration enhancer. Also disclosed herein are uses of the compositions for a method of treatment, comprising the administration of the composition to at least a portion of skin on a subject in need thereof. The compositions may be formulated for cosmeceutical purposes excluding skin malignancies.

### CROSS-REFERENCE TO RELATED APPLICATIONS

The present application claims priority from Australian Provisional Patent Application No. 2022901602, filed on 10 Jun. 2022, and Australian Provisional Patent Application No. 2023900867, filed on 28 Mar. 2023, the contents of each are incorporated herein by reference in their entirety.

### TECHNICAL FIELD

The present disclosure generally relates to compositions and methods for topical delivery of bioactive compounds. More specifically, the present disclosure relates to compositions comprising bioactive compounds formulated for topical administration, methods of application and/or administration, and uses thereof. The compositions may comprise microparticles, at least one bioactive compound, and optionally a penetration enhancer. The compositions may comprise bioactive compounds such as hormonal modulators, and the compositions may be formulated for cosmetic or pharmaceutical purposes, excluding skin malignancies.

### BACKGROUND

Skin is a structurally complex, relatively thick membrane that provides an effective barrier to the entry of any substances into the body. In order to be delivered into or through the skin, a drug molecule must first penetrate the stratum corneum and any material on its surface. The molecule must then at least penetrate the epidermis, while reaching the blood stream or lymph channels would require passing the dermis and/or the capillary walls. To be so absorbed, molecules must overcome a different resistance to penetration in each type of tissue. Transport across the skin membrane is thus a complex phenomenon. However, it is the cells of the stratum corneum that present the primary barrier to absorption of topical compositions. The stratum corneum is a thin layer of dense, highly keratinized cells, approximately 10-15 microns thick, over most of the body.

One technique for delivering bioactive compounds across the skin is transdermal delivery. The term ‘transdermal delivery’ is used for the diffusion of a solute through the different layers of the skin to reach the systemic circulation and elicit a therapeutic effect. Active and passive approaches have been used to optimise transdermal delivery. In the active approach, drug delivery is enhanced using physical methods. In the passive approach, the drug or the formulation is optimised to improve skin permeation. However, for a drug to be delivered through the skin by passive transport, it must be lipophilic and have a molecular weight <500 Da, which can limit the passive transdermal delivery to smaller drugs. Transdermal delivery of drugs offers certain advantages including eliminating the need for intravenous, subcutaneous or intramuscular injections, avoidance of drug degradation in the gastrointestinal tract, retention of the drug at the therapeutic concentration for a prolonged period, minimisation of dosing frequency, improved patient compliance, and potential ability to terminate the drug delivery by removing a transdermal patch.

Commonly used transdermal delivery systems include topical creams and hypodermic needles (which can be used to deliver a drug directly to the dermis). However, topical creams have limited penetration, while the hypodermic needles generally cause pain, may cause damage to skin or bleeding at the site of insertion, and are therefore less accepted by patients.

Recent techniques for transdermal delivery include transdermal patches and microneedle arrays. However, and transdermal patches may be limited to delivery of small molecules as they have to diffuse through several layers before reaching the systemic circulation, while microneedle drug delivery systems require supporting structures such as arrays, which add to the complexity and cost of manufacture and use of such devices. Another disadvantage of transdermal patches and microneedle arrays is that their size and shape limits the delivery of bioactive compounds to accessible areas of skin with pre-determined surface area, and reduced patient comfort due to wearing of the patch or microneedle array.

In particular, microneedle arrays may present discomfort and poor patient acceptance of this mode of delivery in patients with sensitive or very thin skin, or in case of administration to sensitive skin areas such as genitalia.

Further, delivery via transdermal patches is particularly unsuited for avoiding systemic administration of bioactive compounds, as the slow diffusion of deliverable small molecules results in these drugs reaching systemic circulation instead of predominantly saturating the receptors that are localised in the intracutaneous regions of drug penetration and deposition at the application site, which can lead to unwanted systemic side effects.

Accordingly, there is a need for means of topical delivery of bioactive compounds across the stratum corneum that deliver the compounds primarily locally rather than systemically.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The present disclosure relates to compositions that may provide for the advantageous topical delivery of certain bioactive compounds. The compositions disclosed herein comprise one or more bioactive compounds and microparticles, which may be engineered to preferentially penetrate outer parts of a biological barrier such as the skin, for example the stratum corneum of the skin, to enable topical administration of the one or more bioactive compound for a local effect, and minimise or avoid a systemic effect. The compositions disclosed herein can be administered, for example, by applying a force to a composition on a surface of the skin so that at least a fraction of the composition penetrates the stratum corneum of the skin to facilitate a local delivery of the one or more bioactive compound. The compositions of the present disclosure may enable enhanced topical delivery of the one or more bioactive compounds with limited or reduced systemic entry.

Accordingly, in one aspect of the disclosure, there is provided a composition comprising: a plurality of microparticles, at least one bioactive compound, wherein the at least one bioactive compound is optionally a coating on at least a portion of the plurality of microparticles, and an optional penetration enhancer, wherein the composition is formulated for topical application. In one embodiment, the plurality of microparticles, the at least one bioactive compound, and the optional penetration enhancer may be applied topically individually or in combinations thereof, in situ, for example applied to a subject.

There is also provided a composition comprising: a plurality of microparticles, at least one bioactive compound, wherein the at least one bioactive compound is a coating on at least a portion of the plurality of microparticles, and an optional penetration enhancer, wherein the composition is formulated for topical application.

There is also provided a composition comprising: a plurality of microparticles; at least one bioactive compound, wherein the at least one bioactive compound is optionally a coating on at least a portion of the plurality of microparticles, and optionally a penetration enhancer, wherein the composition is formulated for topical application, and with the proviso that no bioactive compound, or substantially no bioactive compound, is delivered to the bloodstream following application of the composition to a portion of skin on a subject.

There is also provided a composition comprising: a plurality of microparticles; at least one bioactive compound, wherein the at least one bioactive compound is optionally a coating on at least a portion of the plurality of microparticles, and a penetration enhancer, wherein the composition is formulated for topical application, and with the proviso that no bioactive compound, or substantially no bioactive compound, is delivered to the bloodstream following application of the composition to a portion of skin on a subject.

There is also provided a composition for topical application, comprising a plurality of microparticles, optionally a penetration enhancer, and at least one bioactive compound that is a ligand of the nuclear receptor superfamily. In some embodiments, the composition further comprises a penetration enhancer.

In some embodiments, the composition is formulated for application to skin, optionally the stratum corneum of the skin.

In some embodiments, the at least one bioactive compound is selected from the group consisting of: a therapeutic compound, a cosmeceutical, a pharmaceutical, a nutraceutical, a diagnostic agent, and mixtures thereof.

In some embodiments, the at least one bioactive compound is: a steroid or a corticosteroid, optionally selected from the group consisting of: estradiol, estrone, estriol, 5α-dihydrotestosterone, finasteride, cortisone, spironolactone, or a mixture thereof, minoxidil, minoxidil sulphate, or a derivative or a mixture thereof, a vitamin, optionally selected from vitamin A, B, C, D3, and/or E; or a retinoid, optionally selected from the group consisting of: retinoic acid, tretinoin, and mixtures thereof. Other bioactive compounds may include: 5-aminolevulinic acid hydrochloride, diclofenac sodium, sodium fluorescein, or mixtures thereof.

In some embodiments, the composition comprises a penetration enhancer selected from the group selected from: non-polar solvents, polar protic solvents, aprotic polar solvents, alcohols (optionally polyols), ionic surfactants, non-ionic surfactants solvents, aliphatic hydrocarbons, fatty acids, and mixtures thereof, wherein the penetration enhancer is optionally selected from the group selected from: ethanol, dimethyl sulfoxide, propylene glycol, isopropanol, polyoxyethylene (20) sorbitan monooleate, urea, sodium docecyl sulfate, menthol, limonene, oleic acid, isopropyl myristate, undecanoic acid, and mixtures thereof.

In some embodiments, the microparticles comprise or consist of: silicon, silica, and mixtures thereof. In one embodiment the microparticles do not comprise or consist of titanium. In one embodiment the microparticles do not comprise or consist of a silicon-titanium composite.

In some embodiments, the microparticles are coated with a material, wherein the material is optionally a dry coating and is optionally selected from the group consisting of: alginate, cross-linked alginate, carboxymethyl cellulose, and mixtures thereof.

In some embodiments, the microparticles possess at least one of the following characteristics:
the microparticles are cylindrical, or substantially cylindrical; and/or
the microparticles are elongate, optionally with an aspect ratio of from about 2:1 to about 50:1, or about 5:1 to about 50:1, optionally from about 10:1 to about 20:1; and/or
the microparticles are etched; and/or
the microparticles are hollow, solid or a mixture thereof, and/or
at least a portion of the microparticles have one or more substantially flat end; and/or
at least a portion of the microparticles have one or more substantially convex ends; and/or
the strength of the microparticles is such as to withstand applied forces of at least 0.1 Newtons; and/or
the microparticles are substantially uniform in size.

In some embodiments, the composition is in the form of a liquid, gel, ointment, cream, lotion, paste, salve, spray, or any mixture thereof.

In some embodiments, the composition further comprises:
at least one solvent, optionally selected from the group consisting of: water, ethanol, and mixtures thereof, and/or
gelling and/or crosslinking agent, optionally selected from the group consisting of: carboxymethylcellulose, a salt, and mixtures thereof, and wherein the salt of optionally selected from the group consisting of: calcium chloride, and mixtures thereof.

In some embodiments, the composition as defined herein is provided with the proviso that no bioactive compound, or substantially no bioactive compound, is delivered to the bloodstream following application of the composition to a portion of skin on a subject.

In some embodiments, a composition, a pharmaceutical composition and/or a cosmeceutical composition, as described here, if formulated for local delivery.

There is also provided a pharmaceutical composition comprising the composition as defined herein, and at least one pharmaceutically acceptable excipient.

There is also provided a cosmeceutical composition comprising the composition as defined herein, and at least one excipient, optionally at least one pharmaceutically acceptable excipient.

There is also provided an applicator adapted to administer a composition as defined herein, a pharmaceutical composition as defined herein, or a cosmeceutical composition as defined herein. In some embodiments, the applicator includes a microtextured surface. In some embodiments, the microtextured surface includes angled faces that define spaces for receiving the microparticles during use of the applicator.

There is also provided a method of treating and/or preventing at least one menopause symptom in a subject, the method comprising administering a composition as defined herein, a pharmaceutical composition as defined herein, and/or a cosmeceutical composition as defined herein, to the subject.

There is also provided a use of a composition as defined herein, a pharmaceutical composition as defined herein, or a cosmeceutical composition as defined herein, in the manufacture of a medicament for the treatment and/or prevention of at least one menopause symptom in a subject.

There is also provided a composition as defined herein, a pharmaceutical composition as defined herein, or a cosmeceutical composition as defined herein, for use in the treatment and/or prevention of at least one menopause symptom in a subject.

In some embodiments, the at least one menopause symptom is optionally selected from the group consisting of: hot flushes, night sweats, crawling or itching sensations under the skin, headache, tiredness, urinary frequency, vaginal dryness, and mixtures thereof.

There is also provided a method of hydrating and/or increasing collagen formation in at least a portion of skin on a subject, the method comprising administering a composition as defined herein, a pharmaceutical composition as defined herein, or a cosmeceutical composition as defined herein, to the subject.

There is also provided a use of a composition as defined herein, a pharmaceutical composition as defined herein, or a cosmeceutical composition as defined herein to hydrate and/or increase collagen formation in at least a portion of skin on a subject.

There is also provided a composition as defined herein, a pharmaceutical composition as defined herein, or a cosmeceutical composition as defined herein, for use in hydrating and/or increasing collagen formation in at least a portion of skin on a subject.

There is also provided a method of improving or treating the appearance of skin on a subject comprising administering to the skin an effective amount of a composition as defined herein, a pharmaceutical composition as defined herein, or a cosmeceutical composition as defined herein, to the subject.

There is also provided a use of a composition as defined herein, a pharmaceutical composition as defined herein, or a cosmeceutical composition as defined herein, to improve and/or treat the appearance of skin on a subject.

There is also provided a composition as defined herein, a pharmaceutical composition as defined herein, or a cosmeceutical composition as defined herein, for use in improving or treating the appearance of skin on a subject.

There is also provided a method of treating and/or preventing hair loss on a subject, the method comprising administering a composition as defined herein, a pharmaceutical composition as defined herein, or a cosmeceutical composition as defined herein, to the subject, wherein the composition is formulated for topical application, and wherein the at least one bioactive compound optionally comprises minoxidil or a derivative thereof, minoxidil sulphate or a derivative thereof, finasteride or a derivative thereof, or a mixture thereof.

There is also provided a use of a composition as defined herein, a pharmaceutical composition as defined herein, or a cosmeceutical composition as defined herein, in the manufacture of a medicament for treating and/or preventing hair loss on a subject, wherein the composition is formulated for topical application, and wherein the at least one bioactive compound optionally comprises minoxidil or a derivative thereof, minoxidil sulphate or a derivative thereof, finasteride or a derivative thereof, or a mixture thereof.

There is also provided a composition as defined herein, a pharmaceutical composition as defined herein, or a cosmeceutical composition as defined herein, for use in treating and/or preventing hair loss on a subject, wherein the composition is formulated for topical application, and wherein the at least one bioactive compound optionally comprises minoxidil or a derivative thereof, minoxidil sulphate or a derivative thereof, finasteride or a derivative thereof, or a mixture thereof. In some embodiments, the at least one bioactive compound comprises minoxidil or a derivative thereof, minoxidil sulphate or a derivative thereof, or a mixture thereof. In some embodiments, the hair loss is male balding.

There is also provided a method of treating and/or preventing a hirsutism in a subject, the method comprising administering a composition as defined herein, a pharmaceutical composition as defined herein, or a cosmeceutical composition as defined herein, wherein the composition is formulated for topical application, and wherein the at least one bioactive compound optionally comprises spironolactone or a derivative thereof.

There is also provided a use of a composition as defined herein, a pharmaceutical composition as defined herein, or a cosmeceutical composition as defined herein, in the manufacture of a medicament for treating and/or preventing a hirsutism in a subject, wherein the composition is formulated for topical application, and wherein the at least one bioactive compound optionally comprises spironolactone or a derivative thereof.

There is also provided a composition as defined herein, a pharmaceutical composition as defined herein, or a cosmeceutical composition as defined herein, for use in treating and/or preventing a hirsutism in a subject, wherein the composition is formulated for topical application, and wherein the at least one bioactive compound optionally comprises spironolactone or a derivative thereof. In some embodiments, the at least one bioactive compound comprises spironolactone or a derivative thereof.

There is also provided a method of treating and/or preventing a dermatological condition or disease in a subject, the method comprising administering a composition as defined herein a pharmaceutical composition as defined herein, or a cosmeceutical composition as defined herein, to the subject.

There is also provided a use of a composition as defined herein, a pharmaceutical composition as defined herein, or a cosmeceutical composition as defined herein, in the manufacture of a medicament for treating and/or preventing a dermatological condition or disease in a subject.

There is also provided a composition as defined herein, a pharmaceutical composition as defined herein, or a cosmeceutical composition as defined herein, for use in treating and/or preventing a dermatological condition or disease in a subject. In some embodiments, the dermatological condition or disease is acne.

There is also provided a method of delivering at least one bioactive compound through the stratum corneum of skin on a subject for cosmeceutical purposes, excluding skin malignancies, the method comprising administering a composition as defined herein, or a cosmeceutical composition as defined herein, wherein the bioactive compound is optionally estradiol, estrone, estriol or a retinoid, optionally in the presence of a further hormonal modulator.

There is also provided a use of a composition as defined herein, a pharmaceutical composition as defined herein, or a cosmeceutical composition as defined herein, to deliver at least one bioactive compound through the stratum corneum of skin on a subject for cosmeceutical purposes, excluding skin malignancies, wherein the bioactive compound is optionally estradiol, estrone, estriol or a retinoid, optionally in the presence of a further hormonal modulator.

There is also provided a composition as defined herein, a pharmaceutical composition as defined herein, or a cosmeceutical composition as defined herein, for use in delivering at least one bioactive compound through the stratum corneum of skin on a subject for cosmeceutical purposes, excluding skin malignancies, wherein the bioactive compound is optionally estradiol, estrone, estriol or a retinoid, optionally in the presence of a further hormonal modulator.

In some embodiments, the composition is administered at least once daily for a period of at least 1 month.

In some embodiments, the composition is administered with the aid of an applicator as defined herein.

In some embodiments, the subject is a human.

### BRIEF DESCRIPTION OF DRAWINGS

Embodiments of the present disclosure will be further described and illustrated, by way of example only, with reference to the accompanying drawings in which:

FIG. 1—Disintegration per minute (DPM) of 1 mL of calibration standards versus concentrations, (A) 10-30 μg/mL and (B) 0.5-5 μg/mL. Equation 5 was used to calculate total E2.

FIG. 2—Total E2 concentration versus observed total E2 concentration. Equation 5 was used to calculate total E2.

FIG. 3—DPM of 1.5 mL digested skin spiked with 10 μL of calibration standards versus total E2 concentrations, (A) 100-300 ng/cm² and (B) 5-50 ng/cm⁷.

FIG. 4—Total E2 concentration of E2 spiked with digested skin versus Observed total E2 concentration of E2. (A) 5-300 ng/cm² and (B) 5-150 ng/cm², expected target dose area in skin circled in red. Equation 5 was used to calculate total E2.

FIG. 5—(A) DPM found in one skin biopsy (0.28 cm²) for three E2 concentrations, (B) amount penetrated in one skin biopsy (0.28 cm²) after 90 minutes, and (C) amount calculated on one cm² of the skin by multiplying biopsy amount by 3.5.

FIG. 6—Transepidermal water loss (TEWL) of ex-vivo abdominal human skin (female, 26 years old), before and after treating skin with various chemical penetration enhancers (CPE) formulations containing E2 and E2-14C: (A) with Foroderm and (B) without Foroderm; CPE include ethanol (Eth), dimethyl sulfoxide (DMSO), propylene glycol (PG), isopropyl alcohol (IPA), Tween 80 (T80), urea, sodium dodecyl sulfate (SDS), menthol (Ment), limonene (Lim), oleic acid (OA), isopropyl myristate (IPM) and undecanoic acid (UDA).

FIG. 7—(A) DPM found in one skin biopsy (0.28 cm²) in two tested groups; CPE combined with Foroderm (Foroderm) and CPE applied with applicator (without Foroderm); (B) amount penetrated in one skin biopsy (ng/biopsy) (surface area of 0.28 cm²) after 90 minutes; (C) amount calculated on one cm² of the skin by multiplying biopsy amount by 3.5 (ng/cm²). CPE include ethanol (Eth), dimethyl sulfoxide (DMSO), propylene glycol (PG), isopropyl alcohol (IPA), Tween 80 (T80), urea, sodium dodecyl sulfate (SDS), menthol (Ment), limonene (Lim), oleic acid (OA), isopropyl myristate (IPM) and undecanoic acid (UDA).

FIG. 8—Serum estradiol levels (pmol/L) following Foroderm-mediated topical delivery of estradiol to postmenopausal female patients with extremely thin and fragile skin.

FIG. 9—Applicator head profile (default). This image represents applicator head roughness and height histogram of scanned area (2565.177×2562.142 μm).

FIG. 10—Applicator head profile with ethanol. This image represents applicator head roughness and height histogram of scanned area (2565.177×2562.142 μm).

FIG. 11—Applicator head profile with dimethyl sulfoxide (DMSO). This image represents applicator head roughness and height histogram of scanned area (2565.177×2562.142 μm).

FIG. 12—Applicator head profile with Tween-80. This image represents applicator head roughness and height histogram of scanned area (2565.177×2562.142 μm).

FIG. 13—Applicator head profile with undecanoic acid (UDA). This image represents applicator head roughness and height histogram of scanned area (2565.177×2562.142 μm).

FIG. 14—The representative images of Foroderm treated areas with an applicator in ex vivo human skin. Area 1=Rh 6G only, Area 2=Foroderm+applicator+Rh6G, Area 3=Foroderm+applicator+Rh6G, Area 4=Rh 6G only, Area 5=Foroderm+applicator+Rh6G, Area 6=Rh 6G only.

### DETAILED DESCRIPTION

The present disclosure describes the following various non-limiting examples.

### General Definitions and Terms

In the following description, reference is made to the accompanying drawings which form a part hereof, and which is shown, by way of illustration, several embodiments. It is understood that other embodiments may be utilised and structural changes may be made without departing from the scope of the present disclosure.

With regards to the definitions provided herein, unless stated otherwise, or implicit from context, the defined terms and phrases include the provided meanings. In addition, unless explicitly stated otherwise, or apparent from context, the terms and phrases below do not exclude the meaning that the term or phrase has acquired by a person skilled in the relevant art. The definitions are provided to aid in describing particular embodiments, and are not intended to limit the claimed invention, because the scope of the invention is limited only by the claims. Furthermore, unless otherwise required by context, singular terms shall include pluralities and plural terms shall include the singular.

All publications discussed and/or referenced herein are incorporated herein in their entirety.

Throughout this disclosure, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the examples, steps, features, methods, compositions, formulations, articles, applications, coatings, and processes, referred to or indicated in this specification, individually or collectively, and any and all combinations or any two or more of said steps or features.

The term “and/or”, e.g., “X and/or Y” shall be understood to mean either “X and Y” or “X or Y” and shall be taken to provide explicit support for both meanings or for either meaning.

Unless otherwise indicated, the terms “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 4.5, and 5, unless where integers are required or implicit from context. This applies regardless of the breadth of the disclosed range. Where specific values are required, these will be indicated in the specification.

As used herein the term “topical composition” refers to a composition which is suitable for application to the surface of a body part, or a localised area of the body of a subject. In one embodiment, the surface of a body part comprises skin. In some embodiments, the surface of a body part comprises mucous membranes, vagina, oral cavity, lesions, scars, wound sites, and the like. Herein, a composition or a topical composition may be in the form of, including, but not limited to: a cream, emulsion, gel, ointment, lotion, levigate, solution, paste, salve, milk, spray, suspension, foam, or the like. In one embodiment a composition or topical composition may be in the form of a bioadhesive. In one embodiment the composition or topical composition may be component of an impregnated pad and/or article, or the like.

In some embodiments, the disclosure describes “topical application” of a composition. As defined herein, “topical application” is application of the composition to the surface of a body part, or a localised area of the body of a subject, and can involve applying and rubbing into the surface.

As used herein, the term “cosmetic” refers to an item that is an article intended to be rubbed, poured, sprinkled, or sprayed on, introduced into, or otherwise applied to the human body, for cleansing, beautifying, promoting attractiveness, or altering the appearance.

Herein “cosmeceutical” refers to a composition that may provide both cosmetic and/or pharmaceutical effects. Whilst a cosmetic may temporarily treat a symptom of a condition on an area of skin, a cosmeceutical may treat one or more underlying conditions relating to the symptom. In an example, a cosmeceutical encompassed by the present disclosure re-grows a subject's hair.

The recipients of a composition of formulation as defined herein may be referred to with the interchangeable terms “recipient” “individual”, “patient”, and “subject”. These three terms are used interchangeably and refer to any human or animal (unless indicated otherwise), as defined herein. In an example, the subject is a human. In an example, the human is an adult.

The recipients of a composition or formulation as defined herein can be a human or a non-human animal, male or female. In an embodiment the subject is a human subject. In one embodiment the subject is female. In another embodiment the subject is female and suffers from menopause. In another embodiment the subject is male. In an embodiment the male subject suffers from hair loss. In another embodiment the subject suffers from hirsutism (excessive hair) in non-scalp regions of their body. In an example, the subject with hirsutism has polycystic ovary syndrome.

As used herein, the term “skin condition” encompasses human and animal conditions, disorders, or diseases affecting skin. Such skin conditions may include, but are not limited to, conditions involving the epidermis, dermis (including connective tissue, sebaceous glands and hair follicles), and the subcutaneous tissue (hypodermis).

As used herein, “skin rejuvenation” means a process of reducing, diminishing, retarding or reversing one or more signs of skin aging. For instance, common signs of skin aging include, but are not limited to, appearance of fine lines or wrinkles, thin and transparent skin, loss of collagen, or loss of underlying fat. According to the present disclosure, one or more of the above signs of skin aging may be reduced, diminished, retarded or even reversed by the compositions and methods of the present disclosure. In certain embodiments, one or more signs of skin aging suitable to be reduced, diminished, retarded or even reversed by the compositions and methods of the present disclosure, are associated with the onset of menopause.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term “consisting essentially of” is intended to exclude elements which would materially affect the properties of the claimed composition.

The terms “comprising”, “comprise” and “comprises” herein are intended to be optionally substitutable with the terms “consisting essentially of”, “consist essentially of”, “consists essentially of”, “consisting of”, “consist of” and “consists of”, respectively, in every instance.

Herein the term “about” encompasses a 10% tolerance in any value or values connected to the term.

Herein “weight %” may be abbreviated as “wt %” or “wt. %”.

Unless described directly, or clear from context, herein the concentration of a component by percentage may refer to w/w %, v/v % or w/v %.

### Compositions

Disclosed herein is a composition comprising:
a plurality of microparticles;
at least one bioactive compound, wherein the at least one bioactive compound is optionally a coating on at least a portion of the plurality of microparticles; and
optionally a penetration enhancer,
   wherein the composition is formulated for topical application.

Also disclosed herein is a composition comprising:
a plurality of microparticles;
at least one bioactive compound (optionally as a coating on at least a portion of the at least one microparticles); and
optionally a penetration enhancer,
   wherein the composition is formulated for topical application, and with the proviso that no bioactive compound, or substantially no bioactive compound, is delivered to the bloodstream following application of the composition to a portion of skin on a subject.

There is also disclosed a composition for topical application, comprising: a plurality of microparticles; optionally a penetration enhancer; and at least one bioactive compound that is a ligand of the nuclear receptor superfamily.

The compositions disclosed herein are not particularly limited so long as they are suitable for topical administration (e.g., application to the skin of a subject).

Accordingly, the composition may be formulated for any form known in the art for topical application. Example forms include, but are not limited to: a liquid, aqueous solution, alcohol solution, emulsion, foam, gel, ointment, lotion, cream, wax, paint, paste, powder, levigate, bioadhesive, salve, milk, spray, suspension, or the like. The compositions disclosed herein may be formulated for application to various body surfaces including skin, mucous membranes such as oral mucosa, vaginal or cervical epithelium or anal canal, as well as scalp and hair.

In one embodiment, the composition disclosed herein is in the form of a liquid.

The composition may comprise one or more solvents. Any suitable solvent or combinations of solvents may be used in the composition. Example of suitable solvents include, but are not limited to: water, alcohols, acetone, methyl alcohol, ethanol, isopropanol, butyl alcohol, ethyl acetate, dimethyl isosorbide, propylene glycol, glycerol, ethylene glycol, polyethylene glycol, diethylene glycol monoethyl ether or mixtures thereof. In some embodiments, the solvent includes ethanol. In some embodiments, the solvent includes a mixture of water and ethanol. In some embodiments, the solvent includes isopropyl alcohol. Further examples of suitable solvents include lanolin alcohols, fatty alcohols (e.g., cetearyl alcohol, cetyl alcohol), glycols (e.g., propylene glycol, polypropylene glycols), oils and waxes (e.g., mineral oils, paraffins), isopropyl myristate, or oleic acid.

The compositions herein may comprise one or more solvents with the same and/or different boiling points. The solvents may be selected based on a number of factors, including, but not limited to: toxicity, density, viscosity, dielectric constant, dipole moment, and/or melting and boiling point. “Solvents” can be broadly classified as low, middle or highly boiling according to boiling temperature at 1 bar: low boiling: boiling ranges below 100° C.; medium boiling: boiling ranges between 100° C. and 150° C.; high boiling: boiling ranges above 150° C. A low boiling solvent is a highly volatile solvent whereas a high boiling solvent is a solvent with a poor inclination to evaporate so that it can be defined as a low volatile solvent. One or more solvents may have a boiling point of about, at least about, or less than about: 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., or 150° C. For example at least solvent may have a boiling point in the range of about 40° C. to about 100° C. and/or a high vapour pressure.

In one embodiment, the solvent is a mixture of water and ethanol, such as a: 95:5 v/v, 90:10 v/v, 85:15 v/v, 80:20 v/v, 75:25 v/v, 70:30 v/v, 65:35 v/v, 60:40 v/v, 55:45 v/v, 50:50 v/v, 45:55 v/v, 40:60 v/v, 35:65 v/v, 30:70 v/v, 25:75 v/v, 20:80 v/v, 15:85 v/v, 10:90 v/v, or 5:95 mixture of water and ethanol.

The one or more solvents may be in an amount of between about 2% and about 99.99% based on total weight of the composition. The one or more solvents can be provided in an amount (as a weight % based on total weight of the composition) of less than about: 99.99, 99.95, 99.9. 99.5, 99, 98, 95, 90, 85, 80, 75, 70, 65, 60, 55, 50, 45, 40, 35, 30, 25, 20, 15, 10, 5, or 2. The one or more solvents can be provided in an amount (as a weight % based on total weight of the composition) of at least about: 2, 5, 10, 15, 20, 25, 30, 35, 40, 45, 50, 55, 60, 65, 70, 75, 80, 85, 90, 95, 98, 99, 99.5, 99.9, 99.95 or 99.99. The one or more solvents can be in a range provided by any two of these upper and/or lower amounts. In some embodiments, the one or more solvents is provided in an amount of about: 15, 16, 17, 18, 19, 20, 21, 22, 23, 24, 25, 26, 27, 28, 30, 31, 32, 33, 34, 35, 36, 37, 38, 39 or 40 wt % based on total weight of the composition.

It will be appreciated that the one or more solvents may be suitably one or more substances which may also act as diluents, penetration enhancers, viscosity enhancers, or film formers. For example, the one or more solvents may be provided as a penetration enhancing compound. The carrier material may also include encapsulating material.

In one embodiment, the composition comprises water. In one embodiment, any water added to the composition is pharmaceutical grade water, for example pharmaceutically acceptable water according to the British Pharmacopeia.

In some embodiments, the composition comprises a buffer. For example, the buffer may be selected from the group comprising, but not limited to: glycerol, glutathione, phosphate-based buffer (e.g., sodium and potassium hydrogen phosphate buffers), acetate buffer, phosphate buffer, citrate buffer, TRIS buffer, maleate buffer, succinate buffer, histidine buffer, amino acids (e.g., histidine, arginine, and glycine), or mixtures thereof. Other non-limiting examples of suitable buffering agents may include, but are not limited to: sodium citrate, magnesium carbonate, magnesium bicarbonate, calcium carbonate, and calcium bicarbonate.

One or more pH buffering agents may be present and, when dissolved in an aqueous component of the composition, may provide a pH in the range of about 5 to about 7. For example, the pH may be about, at least about, or less than about: pH: 5.5, 6, 6.5, 6.6, 6.7, 6.8, 6.9, 7.0, 7.1, 7.2, 7.3, 7.4, or 7.5. The pH may be in a range of any one of the stated values.

In some embodiments, the buffer is present in the composition and is provided in an amount from about 0.01 to about 15 wt. % based on total weight of the composition. The buffer can be provided in an amount (as a weight % based on total weight of the composition) of less than about: 15, 10, 5, 1, 0.5, 0.1, 0.05, or 0.01. The buffer can be provided in an amount (as a weight % based on total weight of the composition) of at least about: 0.01, 0.05, 0.1, 0.5, 1, 5, 10, or 15. The buffer can be in a range provided by any two of these upper and/or lower amounts.

In another embodiment, the composition comprises a mixture of solvents, for example water and ethanol.

In some embodiments, the composition disclosed herein is in the form of an aqueous or alcohol solution.

Solutions are generally homogeneous mixtures and may be prepared by dissolving one or more components of the composition in one or more solvents described herein (e.g., aqueous soluble solvent), such that the components of the composition are dispersed among those of the one or more solvents. The solution may comprise other pharmaceutically and/or cosmetically acceptable chemicals to buffer, stabilise or preserve the components of the composition. Examples of the one or more solvents that may be used in preparing topical compositions include, but are not limited to: water, alcohols (e.g., ethanol, isopropyl alcohol, benzyl alcohol), lanolin alcohols, fatty alcohols (e.g., cetearyl alcohol, cetyl alcohol), glycols (e.g., propylene glycol, polypropylene glycols), oils and waxes (e.g., mineral oils, paraffins), isopropyl myristate, or oleic acid. These can be applied in any manner, such as spraying them on at least a portion of skin, or applying or painting them on at least a portion of skin.

In some embodiments, the composition disclosed herein is formulated as a liquid spray.

Compositions in the form of a liquid, solution or a liquid spray can be applied in any suitable manner, such as painting or spraying them on the body surface. A solution may be applied to the surface with a dropper, swab, sprayer or the like.

In some embodiments, the composition disclosed herein is formulated as a gel, liquid, cream, lotion, or ointment.

In some embodiments, the composition comprises a viscosity enhancer, and/or film former. A viscosity enhancer increases the viscosity of the composition so as to inhibit its spread beyond the site of application. Any suitable viscosity increasing agent may be used, and combinations of viscosity increasing agents may also be used for formulating the compositions. In some embodiments, a polymeric portion of the viscosity increasing agent may be present and may act as a visco-elastic substance and may retain the composition at the site of application. Examples of viscosity increasing agents include, but are not limited to: co-polymers of carboxymethylcellulose and acrylic acid, N-vinylpyrrolidone, polyalkylene glycols (e.g., poly(ethylene glycol)), polyalkylene oxides (e.g., polyethylene oxide), polyvinyl alcohols, polyvinylpyrrolidone, polysiloxanes, poly(vinyl acetates), cellulose, derivatised celluloses, alginates, copolymers thereof and blends thereof. For example, a viscosity agent may be hydroxypropylcellulose, such as Klucel® hydroxypropylcellulose. It will be appreciated that a viscosity enhancer, as described herein, may also have gelling and/or crosslinking properties.

A film former, when it dries, forms a protective film over the site of application. A film former may act additionally as a solvent. Solutions that dry to form a film are sometimes referred to as paints.

In one embodiment, the composition disclosed herein is in the form of a gel, for example a topical gel.

Gels, hydrogels are semisolid, suspension-type systems. Single-phase gels comprise organic macromolecules distributed substantially uniformly throughout the carrier liquid, which is may be aqueous, but also may comprise an alcohol, and optionally an oil.

In some embodiments, the composition comprises at least one gelling and/or crosslinking agent optionally selected from crosslinked acrylic acid polymers such as the “carbomer” family of polymers, e.g., carboxypolyalkylenes that are commercially available as Carbopol®. Hydrophilic polymers may also be used. For example: polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or mixtures thereof.

In some embodiments, the composition comprises at least one gelling and/or crosslinking agent selected from, but not limited to: carboxymethylcellulose, a salt, and mixtures thereof. Examples of salts include calcium chloride. Other salts may include, but are not limited to, inorganic acid salts such as hydrochloride, hydrobromide, sulfate, phosphate and the like; organic acid salts such as: formate, acetate, trifluoroacetate, maleate, tartrate and the like; sulfonates such as: methanesulfonate, benzenesulfonate, p-toluenesulfonate and the like; amino acid salts such as arginate, asparaginate, glutamate and the like; metal salts such as sodium salt, potassium salt, caesium salt and the like; alkaline earth metals such as calcium salt, magnesium salt and the like; and organic amine salts such as triethylamine salt, pyridine salt, picoline salt, ethanolamine salt, triethanolamine salt, discyclohexylamine salt, N,N′-dibenzylethylenediamine salt and the like.

In some embodiments, the composition comprises at least one carbohydrate, which may also act as a gelling and/or crosslinking agent. A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ, where n is an integer. A carbohydrate can be a monosaccharide, a disaccharide, trisaccharide, oligosaccharide, or polysaccharide. The most basic carbohydrate is a monosaccharide, and may include glucose, galactose, mannose, ribose, arabinose, xylose, and fructose. Disaccharides are two joined monosaccharides. Examples of disaccharides may include sucrose, maltose, cellobiose, trehalose, and lactose. An oligosaccharide may include between three and six monosaccharide units (e.g., raffinose, stachyose), and polysaccharides include six or more monosaccharide units. Examples of polysaccharides may include starch, glycogen, and cellulose. Carbohydrates can comprise modified saccharide units such as 2′-deoxyribose wherein a hydroxyl group is removed, 2′-fluororibose wherein a hydroxyl group is replace with a fluorine, or N-acetylglucosamine, a nitrogen-containing form of glucose (e.g., 2-fluororibose, deoxyribose, and hexose). Carbohydrates can exist in many different forms, for example, conformers, cyclic forms, acyclic forms, stereoisomers, tautomers, anomers, and isomers. For example, the polysaccharide may be selected from the group comprising or consisting of: dextran sulfate, pectin, modified pectin, insoluble 1,3-β-D glucan, micronised 1,3-β-D glucan, soluble 1,3-β-D glucan, phosphorylated 1,3-β-D glucan, aminated 1,3-β-D glucan and carboxymethylated 1,3-β-D glucan, sulfated 1,3-β-D glucan, insoluble 1,3/1,6-β-D glucan, micronised 1,3/1,6-β-D glucan, soluble 1,3/1,6-β-D glucan, phosphorylated 1,3/1,6-β-D glucan, aminated 1,3/1,6-β-D glucan and carboxymethylated 1,3/1,6-β-D glucan or sulfated 1,3/1,6-β-D glucan.

In some embodiments, the at least one gelling and/or crosslinking agent, and/or the at least one carbohydrate, is used as a coating excipient for the microparticles in the composition.

In some embodiments, the at least one carbohydrate comprises an alginate-based compound, for example alginic acid, alginic acid derivatives and the salts of alginic acid (alginates), or of the said derivatives. Alginic acid (sometimes referred to in the literature as “alginate” or “algin”), is an anionic polysaccharide distributed widely in the cell walls of algae. Alginic acid is a linear copolymer with homopolymeric blocks of (1-4)-linked β-D-mannuronate (M) and its C-5 epimer α-L-guluronate (G) residues, respectively, covalently linked together in different sequences or blocks. The monomers can appear in homopolymeric blocks of consecutive G-residues (G-blocks), consecutive M-residues (M-blocks) or alternating M and G-residues (MG-blocks). Alginic acid is capable of forming water-soluble salts (alginates) with alkali metals such as sodium, potassium or lithium, substituted cations of lower amines and of ammonium such as methylamine, ethanolamine, diethanolamine or triethanolamine. The alginate-based compound may be water-soluble.

Non-limiting examples of salts of alginic acid (alginates) include, but are not limited to: sodium alginate, potassium alginate, magnesium alginate, calcium alginate, propylene glycol alginate, ammonium alginate, triethanolamine alginate, or mixtures thereof. In some instances, the compositions may include alginic acid, sodium alginate, potassium alginate, or a mixture thereof, because they are particularly water-soluble.

The amount of the gelling and/or crosslinking agent, and/or the at least one carbohydrate, in the composition may vary but may be about, or at least about 0.1 to about 20 wt. %, based on the total weight of the composition. The total amount of the gelling and/or crosslinking agent and/or the at least one carbohydrate in the composition may be: about 0.1 to about 15 wt. %, about 0.1 to about 10 wt. %, about 0.1 to about 5 wt. %, about 0.1 to about 4 wt. %, about 0.1 to about 3 wt. %, about 0.1 to about 2 wt. %. The amount may be: about 0.5 to about 20 wt. %, about 0.5 to about 15 wt. %, about 0.5 to about 10 wt. %, about 0.5 to about 5 wt. %, about 1 to about 20 wt %, about 1 to about 15 wt. %, about 1 to about 10 wt. %, or about 1 to about 5 wt. %, based on the total weight of the composition. In some embodiments, the amount of the gelling and/or crosslinking agent, and/or the at least one carbohydrate, in the composition, is: about 0.1 wt % to about 5 wt %, about 0.2 wt % to about 4 wt %, about 0.3 wt % to about 3 wt %, about 0.4 wt % to about 2 wt %, about 0.5 wt % to about 1 wt %, based on the total weight of the composition.

The gel may be formulated to have a relatively high level of volatile solvent or excipients. In some embodiments, the percent of volatiles by weight is greater than about 55% or any range therein. In particular embodiments, the percent of volatiles by weight is about: 55 wt %, 60 wt %, 65 wt %, 70 wt %, 75 wt %, 80 wt %, or 85 wt %.

In another embodiment, the composition disclosed herein is formulated as a cream.

Cream bases are water-washable, and may comprise an oil phase, an emulsifier, and an aqueous phase. The oil phase, also called the “internal” phase, may comprise petrolatum and a fatty alcohol such as cetyl or stearyl alcohol. The aqueous phase usually, although not necessarily, exceeds the oil phase in volume, and may comprise a humectant. The emulsifier in a cream formulation may comprise a non-ionic, anionic, cationic or amphoteric surfactant.

In another embodiment, the composition disclosed herein is formulated as a lotion.

Lotions are preparations to be applied to the skin surface without friction, and may be liquid or semi-liquid preparations in which one or more components of the composition are present in a water or alcohol base. Lotions are usually suspensions of solids, and may comprise a liquid oily emulsion of the oil-in-water type. Lotions can be used for treating large body areas, because of the ease of applying a more fluid composition. It is generally necessary that the insoluble matter in a lotion be finely divided.

Lotions may comprise suspending agents to produce better dispersions as well as compounds useful for localising and holding the one or more components of the composition in contact with the skin, e.g., methylcellulose, sodium carboxymethyl-cellulose, or the like.

In another embodiment, the composition disclosed herein is formulated as an ointment.

Ointments are semisolid preparations that may be based on petrolatum or other petroleum derivatives. The specific ointment base to be used, as will be appreciated by those skilled in the art, is one that will provide for a number of desirable characteristics, e.g., emolliency or the like. An ointment base is generally inert, stable, non-irritating, and non-sensitising. Ointment bases are grouped in four classes: oleaginous bases; emulsifiable bases; emulsion bases; and water-soluble bases (see Remington: The Science and Practice of Pharmacy, 19^{th} Ed. (Easton, PA: Mack Publishing Co., 1995), at pages 1399-1404). Oleaginous ointment bases include, for example, vegetable oils, fats obtained from animals, and semisolid hydrocarbons obtained from petroleum. Emulsifiable ointment bases, also known as absorbent ointment bases, comprise little or no water and include, for example, hydroxystearin sulfate, anhydrous lanolin, and hydrophilic petrolatum. Emulsion or microemulsion ointment bases are either water-in-oil (W/O) emulsions, biocontinuous, or oil-in-water (O/W) emulsions, and include, for example, acetyl alcohol, glyceryl monostearate, lanolin, and stearic acid. Water-soluble ointment bases are prepared from polyethylene glycols of varying molecular weight.

In some embodiments, the composition disclosed herein is in the form of a paste or a powder.

In some embodiments, the composition includes at least one water repelling agent, also referred to as a water repellent. Examples of water repelling agents include silicones, such as cyclomethicone, dimethicone, simethicone, C₂₆-C₂₈ alkyl dimethicone, C₂₆-C₂₈ alkyl methicone, polyphenylsisquioxane, trimethylsiloxysilicate and crosspolymers of cyclopentasiloxane and dimethicone/vinyltrimethylsiloxysilicate, and blends thereof.

In one embodiment, the composition disclosed herein is formulated as a paste. Pastes are semisolid forms in which the compositions comprise a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

The composition disclosed herein may be formulated for application to skin, optionally the stratum corneum of the skin, of a subject.

Herein the composition disclosed herein may be formulated as a cream, lotion, gel, ointment, paste or the like, which may be spread on the surface of the skin of a subject. The composition, which may be in the form of a liquid or a solution, may be applied in the same way, but may be applied with a dropper, swab, sprayer, applicator or the like, and carefully applied to the subject (e.g., to affected areas or areas in need thereof). Topical application of the composition disclosed herein to the surface of a body part, or a localised area of the body of a subject, can involve applying and rubbing into the surface. The compositions disclosed herein can be applied directly to the target location, for example in the form of a topical composition such as a liquid, gel or a cream, or as a part of a dressing or a bandage, or a patch. In one embodiment, the topical composition excludes a patch form. In another embodiment, the topical composition does not comprise a hydrogel. In another embodiment, the topical composition does not form part of a patch.

The composition disclosed herein may be formulated as a cosmetic, a cosmeceutical and/or a pharmaceutical composition, optionally with one or more excipients.

Also disclosed herein is a pharmaceutical composition comprising the composition as defined herein, and at least one pharmaceutically acceptable excipient.

Also disclosed herein is a cosmeceutical composition comprising the composition as defined herein, and optionally at least one pharmaceutically or cosmeceutically acceptable excipient.

Also disclosed herein is a cosmetic composition comprising the composition as defined herein, and optionally at least one cosmetically acceptable excipient.

It will be appreciated that the compositions, pharmaceutical composition, and/or cosmeceutical compositions disclosed herein may comprise one or more additional additives known in the art, for example one or more excipients.

The composition, pharmaceutical composition, and/or cosmeceutical composition disclosed herein may also comprise conventional excipients well known to those skilled in the art. Examples of other components, which may also be an excipient, includes, but is not limited to: colouring agents, surfactants, preservatives, antioxidants, acidity regulators, thickeners, stabilisers, emulsifiers, osmotic enhancers, pH modifiers, fragrances, extenders, suspending, stabilising and/or dispersing agents, and mixtures thereof.

Excipients for use in the compositions disclosed herein are well-known in the art and examples may be found in the Handbook of Pharmaceutical Excipients (Rowe, R. C. et al., APhA Publications; 5^{th} ed., 2005). Classes of excipients include, but are not limited to: waxes, emollients, thickening agents/viscosity increasing agents, humectants, pH modifiers, water repelling agents, anti-foaming agents, surfactants, solubilisers, wetting agents, penetration enhancers, antioxidants, and solvents. The excipients may also be present in the topical composition at any suitable concentration. In some embodiments, the composition includes excipients at a concentration in a range from about 1 to about 99.99 weight percent.

Further examples of excipients for formulating the compositions disclosed herein include any suitable humectant or combination of humectants. Examples include, but are not limited to: glycols, such as diethylene glycol monoethyl ether, glycerols; sugar polyols, such as sorbitol, xylitol and maltitol; polyols such as polydextroses; quillaia, urea, and blends thereof. In some embodiments, the humectant includes an alkylene glycol, such as hexylene glycol.

In addition, other specific skin-benefit actives such as, but not limited to: sunscreens, skin protectant agents, skin-soothing agents, moisturisers, skin-lightening agents, skin tanning agents, whitening agents, agents to reduce sun spots, pigmentation and scarring, collagen precursors, lipid and small molecule anti-wrinkle agents and anti-inflammation agents, may also be included in the compositions, pharmaceutical compositions, and/or cosmeceutical compositions disclosed herein.

The compositions disclosed herein may be in a form that permits delivery of one or more bioactive compounds via contact of the compositions disclosed herein on at least a portion of skin on a subject in need thereof. The term “in need” is meant to include both pharmaceutical, or health-related needs (e.g., treating one or more symptoms of menopause), as well as cosmetic and subjective needs (e.g., altering or improving the appearance of the portion of skin; improving skin elasticity, skin hydration, firmness, skin tone, skin colour and/or texture; increasing softness of skin; treatment of formication; strengthening, preventing loss of, and/or regrowing hair).

The compositions disclosed herein may comprise ingredients/excipients, which can be found in topical pharmaceutical or cosmeceutical compositions, including a dermatologically and/or pharmaceutically acceptable carrier, vehicle or medium that is compatible with the tissues to which it will be applied. The term “dermatologically and/or pharmaceutically acceptable,” as used herein, means that the compositions or components thereof so described are suitable for use in contact with these tissues or for use in patients in general without undue toxicity, incompatibility, instability, allergic response, and the like. As appropriate, the compositions disclosed herein may comprise any ingredient conventionally used in the fields under consideration, and particularly in cosmetics and dermatology.

In some embodiments, the compositions disclosed herein comprise one or more ingredients/excipients selected from, but not limited to: an antioxidant, a preservative, a stabiliser, a binder, a compaction agent, a lubricant, a dispersion enhancer and/or a colouring agent, and mixtures thereof. For example, one or more ingredients/excipients may be selected from, but not limited to: antioxidants, preservatives, salts (e.g., NaCl), sugars (e.g., sucrose and trehalose), sugar alcohols (e.g., mannitol and sorbitol), polysaccharides, vitamins, essential oils, rheology modifiers, transdermal compounding bases or gels, lipoic acid, xanthan gums, and surfactants (e.g., polysorbate 80 (Tween 80) and polysorbate 20 (Tween 20)).

In some embodiments, the one or more ingredients/excipients comprises at least one lipid. A “lipid” may comprise fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments the lipid comprises at least one fatty acid selected from: lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), tetracosanoic acid (24:0), and mixtures thereof.

In some embodiments, the one or more ingredients/excipients comprises at least one supplemental mineral or mineral source. Examples of minerals include, but are not limited to: chloride, sodium, calcium, iron, chromium, copper, iodine, zinc, magnesium, manganese, molybdenum, phosphorus, potassium, selenium, and mixtures thereof. Suitable forms of any of the foregoing minerals include, but are not limited to: soluble mineral salts, slightly soluble mineral salts, insoluble mineral salts, chelated minerals, mineral complexes, non-reactive minerals such as carbonyl minerals, and reduced minerals, and mixtures thereof.

In some embodiments, the one or more ingredients/excipients comprises at least one vitamin. The at least one vitamin may be fat-soluble or water soluble vitamins. Suitable vitamins include but are not limited to: vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin. For example, the one or more vitamins may be selected from: cholecalciferol, α-tocopherol, β-tocopherol, δ-tocopherol, γ-tocopherol, α-tocotrienol, β-tocotrienol, δ-tocotrienol, γ-tocotrienol, and mixtures thereof. In one embodiment, the at least one vitamin comprises vitamin A.

Various other ingredients/excipients may be included in the compositions disclosed herein. These include, but are not limited to: antioxidants, astringents, perfumes, preservatives, vitamins, emollients, pigments, dyes, humectants, propellants, and sunscreen agents, as well as other classes of materials whose presence may be pharmaceutically or otherwise desirable. Other non-limiting examples of optional ingredients/excipients are as follows: preservatives such as sorbate; solvents such as isopropanol and propylene glycol; astringents such as menthol and ethanol; emollients such as polyalkylene methyl glucosides; humectants such as glycerine; emulsifiers such as glycerol stearate, PEG-100 stearate, polyglyceryl-3 hydroxylauryl ether, and polysorbate 60; sorbitol and other polyhydroxyalcohols such as polyethylene glycol; sunscreen agents such as octyl methoxyl cinnamate and butyl methoxy benzoylmethane; antioxidants such as ascorbic acid (vitamin C), α-tocopherol (Vitamin E), β-tocopherol, γ-tocopherol, δ-tocopherol, ε-tocopherol, ζ-tocopherol, θ-tocopherol, η-tocopherol, and retinol (vitamin A); essential oils, ceramides, essential fatty acids, mineral oils, vegetable oils (e.g., soya bean oil, palm oil, liquid fraction of shea butter, sunflower oil), animal oils (e.g., perhydrosqualene), synthetic oils, silicone oils or waxes (e.g., cyclomethicone and dimethicone), fluorinated oils (generally perfluoropolyethers), fatty alcohols (e.g., cetyl alcohol), and waxes (e.g., beeswax, carnauba wax, and paraffin wax); skin-feel modifiers; and thickeners and components that can adapt the structure of compositions such as swelling clays and cross-linked carboxypolyalkylenes. For example, the preservative may be methylparaben, methylparaben sodium, propylparaben, propylparaben sodium, potassium sorbate benzalkonium chloride, benzthonium chloride or any mixture thereof. For example, the antioxidant may be ascorbic acid, sodium ascorbate, sodium bisulfite, sodium metabisulfate, curcumin, curcumin derivatives, ursolic acid, resveratrol, resveratrol derivatives, alpha-lipoic acid, monothioglycerol, α-tocopherol, β-tocopherol, γ-tocopherol, δ-tocopherol, ε-tocopherol, ζ-tocopherol, θ-tocopherol, η-tocopherol, retinol, or any mixture thereof. For example, the one or more vitamins may be selected from, but not limited to: cholecalciferol, α-tocopherol, β-tocopherol, δ-tocopherol, γ-tocopherol, α-tocotrienol, β-tocotrienol, δ-tocotrienol, γ-tocotrienol, and mixtures thereof. For example, the one or more essential oils may be selected from the group comprising: rosemary essential oil, birch essential oil, balsam fir essential oil, peppermint essential oil, marjoram essential oil, helichrysum essential oil, thyme essential oil, frankincense essential, clove essential oil, turmeric essential oil, orange essential oil, or any mixture thereof.

The compositions disclosed herein may include materials that condition the skin (for example the upper layers of the skin in the stratum corneum) and keep it soft by retarding the decrease of its water content and/or protect the skin. For example, such conditioners and moisturising agents include, but are not limited to: pyrrolidine carboxylic acid and amino acids; organic antimicrobial agents such as 2,4,4′-trichloro-2-hydroxy diphenyl ether (triclosan) and benzoic acid. Further optional additives include, but are not limited to: anti-inflammatory agents such as acetylsalicylic acid and glycyrrhetinic acid; anti-seborrhoeic agents such as retinoic acid; vasodilators such as nicotinic acid; inhibitors of melanogenesis such as kojic acid; and mixtures thereof.

The compositions disclosed herein can include alpha hydroxyacids, alpha ketoacids, polymeric hydroxyacids, moisturisers, collagen, marine extract, and antioxidants such as ascorbic acid (vitamin C) and/or α-tocopherol (Vitamin E). Sunscreens may also be included. Additional components such as enzymes, herbs, plant extracts, glandular or animal extracts, can be added to the composition.

The compositions disclosed herein may also comprise antimicrobial agents, to prevent spoilage upon storage, i.e., to inhibit growth of microbes such as yeasts and moulds. Suitable antimicrobial agents may be selected from the group comprising or consisting of: the methyl and propyl esters of p-hydroxybenzoic acid (i.e., methyl and propyl paraben), sodium benzoate, sorbic acid, imidurea, and mixtures thereof.

The compositions disclosed herein may also comprise irritation-mitigating additives to minimise or eliminate the possibility of skin irritation or skin damage resulting from the chemical entity to be administered, or other components of the composition. Suitable irritation-mitigating additives include, for example: α-tocopherol; monoamine oxidase inhibitors, particularly phenyl alcohols such as 2-phenyl-1-ethanol; glycerin; salicylates; ascorbates; ionophores such as monensin; amphiphilic amines; ammonium chloride; N-acetylcysteine; capsaicin; and chloroquine. The irritation-mitigating additive, if present, can be incorporated into the composition disclosed herein at a concentration effective to mitigate irritation or skin damage, and may represent not more than about 15 wt. %, or not more than about 5 wt. %, of the composition.

The amounts of these various ingredients/excipients are those conventionally used in the skin care field. In some embodiments, the one or more ingredients/excipients may be present in the compositions disclosed herein and may be provided in an amount from about 0.01 to about 15 wt. % based on total weight of the composition. The one or more ingredients/excipients can be provided in an amount (as a weight % based on total weight of the composition), of less than about: 15, 10, 5, 1, 0.5, 0.1, 0.05, or 0.01. The one or more ingredients/excipients can be provided in an amount (as a weight % based on total weight of the composition) of at least about: 0.01, 0.05, 0.1, 0.5, 1, 5, 10, or 15. The one or more ingredients/excipients can be in a range provided by any two of these upper and/or lower amounts.

In some embodiments, the composition disclosed herein is admixed with an existing or known pharmaceutical and/or cosmeceutical formulation, which may be in the form of an aqueous solution, gel, cream, lotion, emulsion, serum, spray, suspension, an emulsion, or the like.

In some embodiments, the composition disclosed herein comprises:
the microparticles described herein, wherein one or more or all of the following apply:
   the microparticles are present in a concentration of about or at least about 150 mg, 145 mg, 140 mg, 135 mg, 130 mg, 125 mg, 120 mg, 115 mg, 110 mg, 105 mg, 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, or 25 mg per ml of the composition; optionally wherein the microparticles are present in a concentration of about or at least about 50 mg to about 150 mg per ml, optionally about 70 mg to about 100 mg, per ml of the composition; and/or
   the microparticles are elongate and have a length of up to about 150 μm, about 140 μm, about 130 μm, about 120 μm, about 110 μm, about 100 μm, or about 90 μm; optionally wherein the microparticles have a length in a range of about 90 μm to about 150 μm, optionally about 100 μm to about 130 μm; optionally wherein the microparticles have the dimensions of about 5-10 μm diameter at about 100-130 μm length; and/or
   the microparticles are coated with a coating material, wherein the coating material is optionally selected from alginate, cross-linked alginate, or carboxymethyl cellulose, or a mixture thereof;
at least one bioactive compound, optionally wherein at least one bioactive compound is a coating on at least a portion of the microparticles; and/or
optionally a penetration enhancer, optionally selected from: ethanol, dimethyl sulfoxide, propylene glycol, and/or isopropyl myristate.

In some embodiments, the composition disclosed herein comprises:
the microparticles described herein, wherein one or more or all of the following apply:
   the microparticles are present in a concentration of about or at least about 150 mg, 145 mg, 140 mg, 135 mg, 130 mg, 125 mg, 120 mg, 115 mg, 110 mg, 105 mg, 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, or 25 mg per ml of the composition; optionally wherein the microparticles are present in a concentration of about or at least about 50 mg to about 150 mg per ml, optionally about 70 mg to about 100 mg, per ml of the composition; and/or
   the microparticles are elongate and have a length of up to about 150 μm, about 140 μm, about 130 μm, about 120 μm, about 110 μm, about 100 μm, or about 90 μm; optionally wherein the microparticles have a length in a range of about 90 μm to about 150 μm, optionally about 100 μm to about 130 μm; optionally wherein the microparticles have the dimensions of about 5-10 μm diameter at about 100-130 μm length; and/or
   the microparticles are coated with a coating material, wherein the coating material is optionally selected from alginate, cross-linked alginate, or carboxymethyl cellulose, or a mixture thereof, and/or
at least one bioactive compound that is a ligand of the nuclear receptor superfamily; and/or
optionally a penetration enhancer, optionally selected from ethanol, dimethyl sulfoxide, propylene glycol, and/or isopropyl myristate.

In one embodiment, the composition disclosed herein comprises:
the microparticles described herein (optionally present in an amount of about, or at least about: 1 mg, 2 mg, 3 mg, 4 mg, or 5 mg); and/or
ethanol (optionally present in an amount of about, or at least about: 10%, 20%, or 30%); and/or
isopropyl myrisate (optionally present in an amount of about, or at least about: 1%, 2%, 3%, 4%, or 5%); and/or
menthol (optionally present in an amount of about, or at least about: 0.05%, or 0.1%); and/or
estradiol, estrone, estriol or a retinoid, (optionally present in an amount of about, or at least about: 0.0005%, or 0.001%).

In one embodiment, the composition disclosed herein comprises:
the microparticles described herein (optionally wherein the microparticles are elongate and have a length of up to about 150 μm, about 140 μm, about 130 μm, about 120 μm, about 110 μm, about 100 μm, or about 90 μm; optionally wherein the microparticles have a length in a range of about 90 μm to about 150 μm, optionally about 100 μm to about 130 μm; optionally wherein the microparticles have the dimensions of about 5-10 μm diameter at about 100-130 μm length; and/or wherein the microparticles are optionally present in a concentration of about or at least about 150 mg, 145 mg, 140 mg, 135 mg, 130 mg, 125 mg, 120 mg, 115 mg, 110 mg, 105 mg, 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, or 25 mg per ml of the composition; optionally wherein the microparticles are present in a concentration of about or at least about 50 mg to about 150 mg per ml, optionally about 70 mg to about 100 mg, per ml of the composition; and/or wherein the microparticles are coated with a coating material, wherein the coating material is optionally selected from alginate, cross-linked alginate, or carboxymethyl cellulose, or a mixture thereof); and/or
ethanol (optionally present in an amount of about, or at least about: 10%, 20%, or 30%); and/or
isopropyl myrisate (optionally present in an amount of about, or at least about: 1%, 2%, 3%, 4%, or 5%); and/or
menthol (optionally present in an amount of about, or at least about: 0.05%, or 0.1%); and/or
estradiol, estrone, estriol or a retinoid, (optionally present in an amount of about, or at least about: 0.0005%, or 0.001%).

In one embodiment, the composition, pharmaceutical composition or cosmeceutical composition disclosed herein is formulated with the proviso that no bioactive compound, or substantially no bioactive compound, is delivered to the bloodstream following application of the composition to a portion of skin on a subject. For example, in some embodiments, administration of the composition, pharmaceutical composition or cosmeceutical composition described herein results in limited, reduced, substantially reduced, or no systemic absorption or accumulation of one or more of the bioactive compound present in the composition, therefore reducing or avoiding systemic toxicity and/or side-effects. That is, in certain examples, the one or more bioactive compound delivered via the composition, pharmaceutical composition or cosmeceutical composition disclosed herein will be localised to the intracutaneous regions of a subject treated according to the methods disclosed herein.

Compositions of the disclosure can be assessed to confirm limited or no systemic absorption or accumulation of bioactive compounds via various methods. In an example, a composition disclosed herein is administered to a subject before assessing a representative sample from the subject for the presence of the bioactive compound or a biomarker thereof (e.g., a metabolite). Those of skill in the art will appreciate that the most appropriate sample may be dictated by the bioactive being delivered. For example, in certain embodiments, an appropriate sample may be a urine sample. In other embodiments, an appropriate sample may be a blood sample.

In an example, the presence or level of a particular bioactive compound (or a biomarker thereof, e.g., a metabolite), is determined in a sample obtained from a subject (e.g., a urine sample, blood sample, plasma sample, and/or serum sample), optionally wherein the sample is isolated from the subject. For example, the presence or level of a particular bioactive compound (or a biomarker thereof), can be determined in a plasma sample. In another example, the presence or level of the particular bioactive compound (or a biomarker thereof), is determined in a serum sample.

In an example, the presence or level (e.g., serum level), of a particular bioactive compound or a biomarker thereof (e.g., a metabolite), is determined via an analytical method or assay. Analytical methods and assays for determining the presence or level (e.g., serum level), of a bioactive compound or biomarkers thereof will be well familiar to persons skilled in the art. Such methods include, for example, high-performance liquid chromatography (HPLC)/mass spectrometry, HPLC/ultraviolet (UV), mass spectrometry (MS) based methods, gas chromatography (GC)/electron capture detection (ECD), thin-layer chromatography (TLC)/densitometry, electron spin resonance spectrometry, enzyme-linked immunosorbent assay (ELISA), and a variety of immunoassays such as those described in Hermida J et al., Clin Biochem. 2002, 35(3), 251; Zhao X et al., Analyst. 2003, 128(4), 357; Draisci R et al., Analyst. 2001, 126(11), 1942, and Giese R W, J Chromatogr A. 2003, 1000(1-2), 401. For example, endogenous hormone levels, including for example serum estradiol levels, can be measured in serum samples using a range of serum assays, such as radioimmunoassays.

In certain examples, compositions of the disclosure can be assessed to confirm that they direct limited or no systemic absorption or accumulation of bioactive compounds prior to administration.

In other examples, it may be necessary to monitor a subject following administration of a composition disclosed herein. In an example, monitoring comprises assessing a sample obtained from the subject (e.g., a sample isolated from the subject), for the presence or level of a bioactive compound or a biomarker thereof (e.g., a metabolite). Exemplary samples are briefly discussed above but may include a blood sample. Accordingly, in an example, it may be necessary to monitor the blood of a subject periodically to confirm that a bioactive compound has not entered the bloodstream in significant amounts. For example, regular blood samples can be obtained from a treated subject and assessed for the presence of a bioactive compound. Above referenced methods and assays for confirming the presence or level of a particular bioactive compound are equally applicable in such examples.

### Administration

The compositions disclosed herein may be administered by or under the direction of a physician or other health care professional, such as a health care professional skilled in the art of topical treatments. They may be administered in a single treatment or in a series of treatments over time. The concentration and timescale of treatments dependent on a number of factors such as the form and/or concentration of the components of the composition, the subject, the severity of a condition and/or the particular treatment being undertaken on the subject in need thereof.

Herein, ‘administering’ the compositions disclosed herein means applying the compositions to a required body surface area. In some embodiments, ‘applying’ the compositions disclosed herein includes applying the composition to a biological barrier, for example the skin surface, followed by rubbing or massaging the composition onto the biological barrier, for example by a circular motion or linear motions back and forth, such that at least some of the composition penetrates the biological barrier.

In the context of the present disclosure, a “biological barrier” may refer to the skin, the surface of the skin, or a specific part of the skin such as stratum corneum of the skin. Other biological barriers include, for example, mucous membranes such as oral mucosa, vaginal or cervical epithelium or anal canal, as well as scalp, hair and nails.

In some embodiments, the biological barrier is the skin surface. In some embodiments, the biological barrier is the stratum corneum of the skin. In an embodiment, the biological barrier is the stratum corneum of human skin.

In some embodiments, the compositions disclosed herein only penetrate the stratum corneum of the skin. In some embodiments, the compositions disclosed herein only penetrate the stratum corneum and viable epidermis of the skin.

The compositions disclosed herein can be administered, for example, by applying a force to the composition on a biological barrier such as the surface of the skin so that at least a fraction of the composition penetrates the biological barrier, to facilitate a local delivery of the one or more bioactive compounds. For example, the compositions disclosed herein or fractions thereof may only penetrate the stratum corneum of the skin, or the stratum corneum and viable epidermis of the skin, to facilitate a local delivery of the one or more bioactive compounds. In some embodiments, the compositions disclosed herein or fractions thereof only penetrate the stratum corneum of the skin to facilitate a local delivery of the one or more bioactive compounds.

In order to deliver and/or administer a composition as described herein, a force may be applied, for example, by hand or by using an applicator. In some embodiments, the compositions disclosed herein are administered by rubbing or massaging the compositions onto a biological barrier such as the skin by means of the hand or an applicator. In some embodiments, the compositions disclosed herein are rubbed or massaged onto the biological barrier such as the skin by means of an applicator.

In some embodiments, the applicator is a textured applicator.

In some embodiments, the applicator is a microtextured applicator. A microtextured applicator can be of various sizes suitable for desired surface areas, and made of different suitable materials, such as biocompatible and non-allergenic material, and may be manufactured, for example, using 3D printing.

In some embodiments, the applicator is an applicator described in patent application WO 2014/094067 A1, the entire contents of which are incorporated herein by reference.

Following administration, the number of the microparticles of the compositions disclosed herein in the body may decrease over time as they are expelled from the body. The microparticles of the compositions disclosed herein may be completely expelled from the body after about 1, 2 or 3 weeks. The potential mechanism of microparticle elimination could be associated with natural skin turn-over (desquamation). In humans, over approximately three weeks, new skin cells (keratinocytes) are generated within the stratum basale. The keratinocytes migrate upwards forming the cellular viable epidermis. As the cells migrate closer to the surface of the skin they begin to terminally differentiate (lose their cell nuclei) and flatten. This process renews the stratum corneum and aids in the natural removal of foreign bodies within the skin.

### Bioactive Compound

As defined above, the compositions disclosed herein comprise at least one bioactive compound.

Herein, the terms “bioactive compound”, “bioactive agent”, “bioactive molecule” or “biomolecule”, refer to any organic or inorganic therapeutic, prophylactic or diagnostic agent that actively or passively influences a biological system. Such agents may also be referred to as a “drug”.

The compositions disclosed herein may comprise one or more than one bioactive compound. In some embodiments, one bioactive compound is present. In other embodiments, a plurality of bioactive compounds are present, for example 2, 3, 4 or 5 bioactive compounds, or at least 2, 3, 4 or 5 bioactive compounds.

In one embodiment, the at least one bioactive compound forms at least a part of a coating on the microparticles, or has the capability to form at least part of a coating on the microparticles. In an example, the composition comprises microparticles coated with different bioactive compounds. In another example, the microparticles in the composition are coated with the same bioactive compound. In some embodiments, the at least one bioactive compound and the microparticles are formulated as a mixture.

In one embodiment, the at least one bioactive compound in the compositions disclosed herein is coated on at least some of the surfaces of at least some of the microparticles. When the compound is coated on the microparticles, the compound is delivered with the microparticles as they penetrate a biological barrier. The compound can be coated on the surface of at least some of the microparticles by mixing them with the compound and then e.g., freeze or air drying the microparticles.

The at least one bioactive compound may formulated for topical application to a body surface (e.g., the skin), as a single formulation with the microparticles. For example, the formulation may be formed in advance and stored. Alternatively, the formulation may be formed just prior to the application. Advantageously, an existing or known formulation of the at least one bioactive compound may be mixed with the microparticles, either for storage or immediate topical application.

In some embodiments, in the compositions disclosed herein, the at least one bioactive compound is formulated to be applied to a body surface (e.g., the skin), separately from the microparticles, such as after the application of the microparticles. In some embodiments, the at least one bioactive compound may be in the form of an existing or known formulation of the at least one bioactive compound.

Herein, the bioactive compound or plurality of bioactive compounds may be selected by a person skilled in the art for a particular treatment and/or purpose.

In some embodiments, the at least one bioactive compound is selected from, but not limited to: a therapeutic compound, a cosmeceutical, a pharmaceutical, a nutraceutical, a diagnostic agent, a vaccine, a nucleic acid, and mixtures thereof. In some embodiments, the at least one bioactive compound is selected from a therapeutic compound, a cosmeceutical, a pharmaceutical, a nutraceutical, a diagnostic agent, and mixtures thereof.

In some embodiments, the at least one bioactive compound is a hormonal modulator with cosmetic, pharmaceutical and/or cosmeceutical applications. As used herein, “hormonal modulator” refers to an agent that modulate hormone receptors. Hormonal modulators include any hormonal therapy agents including hormone agonists or hormone antagonists, such as for example steroid hormones or vitamins.

In one embodiment, the at least one bioactive compound is a hormonal modulator for cosmeceutical purposes, excluding skin malignancies.

In some embodiments, the at least one bioactive compound is a ligand of the superfamily of ligand-activated nuclear receptor transcription factors and modifiers of ligand or transcription factor function. The nuclear receptors bind and respond to certain steroids and other signalling molecules, such as vitamin D3, thyroid hormone, and retinoids. For example, the at least one bioactive compound may be selected from the list of known nuclear receptor ligands shown in Table 1 of E. R. Weikum et al., “The nuclear receptor superfamily: A structural perspective”, Protein Science, vol. 27, pp. 1876-1892, 2018, the contents of which are incorporated herein by reference.

In some embodiments, the at least one bioactive compound is a steroid. Examples of steroids include, but are not limited to: estradiol, finasteride, corticosteroids, human growth hormone, testosterone phytoestrogens, hormonally active peptides, and mixtures thereof.

In some embodiments, the at least one bioactive compound is a retinoid. As used herein, the term “retinoid” refers to any naturally occurring or synthetic retinoids or derivatives thereof, including any small chemical molecules that act on retinoic acid receptors (RARs). Examples of retinoids include, but are not limited to, vitamin A, retinoic acid, retinol, retinal, tretinoin, tazarotene, tazarotenic acid, adapalene, isotretinoin, and acitretin, or derivatives thereof, and mixtures thereof.

In some embodiments, the at least one bioactive compound is selected from, but not limited to: tretinoin, tazarotene, tazarotenic acid, adapalene, isotretinoin or acitretin, and mixtures thereof.

In some embodiments, the at least one bioactive compound is a vitamin A derivative. In an example, the vitamin A derivative is retinol or retinoic acid. In another example, the at least one bioactive compound is a retinoid. Examples of retinoids include, but are not limited to: tretinoin, retinoic acid, and mixtures thereof. Other examples of bioactive compounds include, but are not limited to: retinyl acetate, retinyl linoleate, retinyl palmitate and retinyl propionate, and mixtures thereof. In an example, the at least one bioactive compound is tretinoin or acitretin. In another example, the at least one bioactive compound is a topical retinoid and/or topical antibiotic and/or benzoyl peroxide.

In one embodiment, the vitamin A derivative is tretinoin, having the following structure:

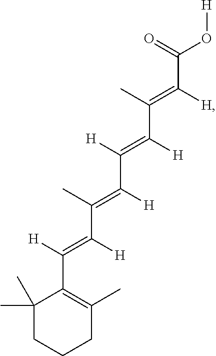

or a pharmaceutically acceptable salt thereof.

Suitable salts of the bioactive compounds described herein and encompassed by the present disclosure include those formed with organic or inorganic acids or bases. As used herein, the phrase “pharmaceutically acceptable salt” or “pharmaceutically effective salt” refers to pharmaceutically acceptable organic or inorganic salts. Exemplary acid addition salts include, but are not limited to: sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)), salts. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. A pharmaceutically acceptable salt may involve the inclusion of another molecule such as an acetate ion, a succinate ion or other counter ion. The counter ion may be any organic or inorganic moiety that stabilises the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. It will also be appreciated that non pharmaceutically acceptable salts also fall within the scope of the present disclosure since these may be useful as intermediates in the preparation of pharmaceutically acceptable salts or may be useful during storage or transport.

Those skilled in the art will also appreciate that many bioactive compounds can form complexes with solvents in which they are reacted or from which they are precipitated or crystallised. These complexes are known as “solvates” and are encompassed by the present disclosure. For example, a complex with water is known as a “hydrate”. As used herein, the phrase “pharmaceutically acceptable solvate”, “pharmaceutically effective solvate”, or “solvate”, refer to an association of one or more solvent molecules and a compound of the present disclosure. Examples of solvents that form pharmaceutically acceptable solvates include, but are not limited to: water, isopropanol, ethanol, methanol, DMSO, ethyl acetate, acetic acid, and ethanolamine.

In some embodiments, the at least one bioactive compound is: vitamin D3, A, B, C and/or E, nicotinamide, hyaluronic acid, bakuchiol, resveratrol, minoxidil, or minoxidil sulphate, or a derivative thereof. In an embodiment, the at least one bioactive compound is vitamin D3, A, B, C and/or E. In an embodiment, the at least one bioactive compound is nicotinamide. In an embodiment, the at least one bioactive compound is hyaluronic acid. In one embodiment, the at least one bioactive compound is bakuchiol. In an embodiment, the at least one bioactive compound is resveratrol. In an embodiment, the at least one bioactive compound is or minoxidil, minoxidil sulphate, or a derivative thereof.

As described herein, the compositions disclosed herein may comprise a combination of any one or more of these bioactive compounds. Accordingly, the compositions disclosed herein may comprise a combination of any one or more of vitamin D3, A, B, C and/or E, nicotinamide, hyaluronic acid, bakuchiol, resveratrol, minoxidil, minoxidil sulphate, or a derivative thereof. In some embodiments, the compositions disclosed herein comprise a combination of any one or more of vitamin D3, A, B, C and/or E, nicotinamide, hyaluronic acid, bakuchiol, and resveratrol. In some embodiments, the compositions disclosed herein comprise a combination of any one or more of vitamin D3, A, B, C and/or E, nicotinamide, hyaluronic acid, bakuchiol, resveratrol and a retinoid, optionally selected from the group consisting of: retinoic acid, tretinoin, and mixtures thereof.

In some embodiments, the at least one bioactive compound is a topical anti-acne agent, such as: adapalene, azelaic acid, benzoyl peroxide, clindamycin and clindamycin phosphate, doxycycline, erythromycin, keratolytics such as salicylic acid and retinoic acid (“Retin-A”), norgestimate, organic peroxides, retinoids such as isotretinoin and tretinoin, sulfacetamide sodium, tazarotene, or a mixture thereof. In some embodiments, the anti-acne agent is selected from, but not limited to: adapalene, azelaic acid, benzoyl peroxide, clindamycin (e.g., clindamycin phosphate), doxycycline (e.g., doxycycline monohydrate), erythromycin, isotretinoin, norgestimate, sulfacetamide sodium, tazarotene, etretinate, or acetretin, or a mixture thereof.

In some embodiments, the at least one bioactive compound is an estrogen hormone or estrogen derivative. Non limiting examples of estrogen hormones or derivatives include, for example: estradiol, estrone, estriol, estetrol, esterified estrogens, 17β-estradiol, estradiol benzoate, 17β-estradiol valerate, estradiol 17β-cypionate, estrone, Estropipate (piperazine estrone sulfate), and 17β-ethinyl estradiol. In some embodiments, the at least one bioactive compound is selected from estradiol, estrone, and estriol, or a derivative thereof.

In any embodiments herein, “derivative” of a bioactive compound encompasses, for example, an ester, ether, amide, carbamate, phosphate, anhydride, or sulphonamide derivative of the compound, and/or a stereoisomer, enantiomer, diastereomer, geometric isomer, racemate, tautomer, zwitterion, active metabolite, or prodrug of the compound, or a pharmaceutically acceptable salt or solvate thereof.

In one embodiment, the at least one bioactive compound is estradiol or a derivative thereof.

Herein a derivative of estradiol may refer to a compound having the following general estradiol structure, wherein one or more available carbon atoms of the general estradiol backbone is substituted with a moiety aside from hydrogen.

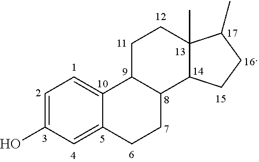 [SMILES: CC1CCC2C3CCc4cc(O)ccc4C3CCC12C]

In some embodiments, the carbon atom at the 1-, 2-, 4-, 6-, 7-, 8-, 9-, 11-, 12-, 14-, 15-, 16-, or 17-position is substituted. In another embodiment, the carbon atom at the 1-, 2-, 4-, 6-, 7-, 11-, 12-, 15-, 16-, or 17-position is substituted. In yet another embodiment, the carbon atom at the 17-position is substituted. In yet another embodiment, one or more carbon atoms are substituted with one or two C₁₋₆alkyl, C₂₋₆alkenyl, or C₂₋₆alkynyl, as permitted by the valency of the carbon atom. In other embodiments, one or more carbon atoms are substituted with C₁₋₆alkyl, such as methyl, ethyl, propyl, butyl, pentyl, or hexyl. In other embodiments, one or more carbon atoms are substituted with C₂₋₆ alkenyl, such as ethenyl, propenyl, butenyl, pentenyl, or hexenyl. In other embodiments, one or more carbon atoms are substituted with C₂₋₆ alkynyl, such as ethynyl, propynyl, butynyl, pentynyl, or hexynyl.

In another embodiment, the at least one bioactive compound is finasteride or a derivative thereof, having the following general structure. Finasteride (i.e., (1S,3aS,3bS,5aR,9aR,9bS,11aS)—N-tert-butyl-9a,11a-dimethyl-7-oxo-1,2,3,3a,3b,4,5,5a,6,9b,10,11-dodecahydroindeno[5,4-f]quinoline-1-carboxamide), is sold under the names Proscar (registered trademark) and Propecia (registered trademark) among others, is a medication for treatment of hair loss and benign prostatic hyperplasia in men.

Herein a derivative of finasteride may refer to a compound having the following general finasteride structure, wherein one or more available carbon atoms of the general finasteride backbone is substituted with a moiety aside from hydrogen.

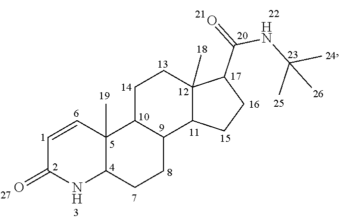 [SMILES: CC(C)(C)NC(=O)C1CCC2C3CCC4NC(=O)C=CC4(C)C3CCC12C]

or a pharmaceutically acceptable salt thereof.

In some embodiments, the carbon atom at the 1-, 4-, 5-, 6-, 7-, 8-, 9-, 10-, 11-, 13-14-, 15-, 16-, or 17-position is substituted. In another embodiment, the carbon atom at the 1-, 6-, 7-, 8-, 10-, 13-14-, 15-, 16-, or 17-position is substituted. In yet another embodiment, one or more carbon atoms are substituted with one or two C₁₋₆alkyl, C₂₋₆alkenyl, or C₂₋₆alkynyl, as permitted by the valency of the carbon atom. In other embodiments, one or more carbon atoms are substituted with C₁₋₆alkyl, such as methyl, ethyl, propyl, butyl, pentyl, or hexyl. In other embodiments, one or more carbon atoms are substituted with C₂₋₆ alkenyl, such as ethenyl, propenyl, butenyl, pentenyl, or hexenyl. In other embodiments, one or more carbon atom is substituted with C₂₋₆ alkynyl, such as ethynyl, propynyl, butynyl, pentynyl, or hexynyl.

In some embodiments, the at least one bioactive compound is minoxidil, or a derivative thereof. Minoxidil (i.e., 2,4-diamino-6-piperidinyl-pyrimidine-3-oxide) having the following structure is an active ingredient of Rogaine (registered trademark), and it is commercially available for treatment and prevention of androgenic alopecia (male pattern baldness and female pattern baldness).

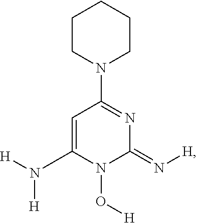

or a pharmaceutically acceptable salt thereof.

In one embodiment, the at least one bioactive compound is minoxidil sulfate (i.e., (2,6-diamino-4-piperidin-1-ylpyrimidin-1-ium-1-yl) sulfate) having the following structure, or a derivative thereof.

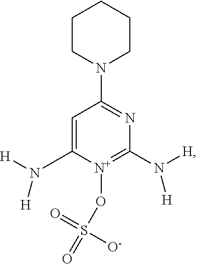

or a pharmaceutically acceptable salt thereof.

In some embodiments, the at least one bioactive compound is a steroid. Non-limiting examples of steroids include estradiol, 5α-dihydrotestosterone, estrone, estriol, and finasteride. In some embodiments, the steroid is a corticosteroid. Non-limiting examples of corticosteroids include cortisone and spironolactone.

In some embodiments, the at least one bioactive compound is spironolactone (i.e., S-[(7R,8R,9S,10R,13S,14S,17R)-10,13-dimethyl-3,5′-dioxospiro[2,6,7,8,9,11,12,14,15,16-decahydro-1H-cyclopenta[a]phenanthrene-17,2′-oxolane]-7-yl]ethanethioate) having the following structure, or a derivative thereof. Spironolactone is marketed under the names Aldactone, Spiractin, Verospiron and others, and is frequently used to treat a variety of dermatological conditions in which androgens play a role. Some of these uses include acne, seborrhea, hirsutism, and pattern hair loss in women.

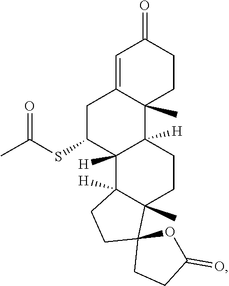 [SMILES: CC(=O)S[C@@H]1CC2=CC(=O)CC[C@]2(C)[C@H]2CC[C@@]3(C)[C@@H](CC[C@@]34CCC(=O)O4)[C@H]12]

or a pharmaceutically acceptable salt thereof.

In some embodiments, the at least one bioactive compound is 5α-dihydrotestosterone (DHT, or 5α-DHT) (i.e., (1S,3aS,3bR,5aS,9aS,9bS,11aS)-1-hydroxy-9a,11a-dimethylhexadecahydro-7H-cyclopenta[a]phenanthren-7-one) having the following structure or a derivative thereof:

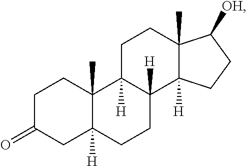

or pharmaceutically acceptable salt thereof.

In some embodiments, the at least one bioactive compound is estrone (i.e., (3aS,3bR,9bS,11aS)-7-hydroxy-11a-methyl-2,3,3a,3b,4,5,9b,10,11,11a-decahydro-1H-cyclopenta[a]phenanthren-1-one) having the following structure or a derivative thereof:

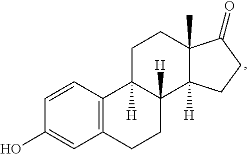

or pharmaceutically acceptable salt thereof.

In some embodiments, the at least one bioactive compound is estriol (i.e., (1R,2R,3aS,3bR,9bS,11aS)-11a-methyl-2,3,3a,3b,4,5,9b,10,11,11a-decahydro-1H-cyclopenta[a]phenanthrene-1,2,7-triol) having the following structure or a derivative thereof:

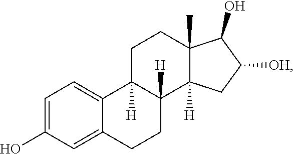

or pharmaceutically acceptable salt thereof.

In some embodiments, the at least one bioactive compound is cortisone (i.e., 17a,21-dihydroxypregn-4-ene-3,11,20-trione; or (1R,3aS,3bS,9aR,9bS,11aS)-1-Hydroxy-1-(hydroxyacetyl)-9a,11a-dimethyl-2,3,3a,3b,4,5,8,9,9a,9b,11,11a-dodecahydro-7H-cyclopenta[a]phenanthrene-7,10(1H)-dione) having the following structure or a derivative thereof:

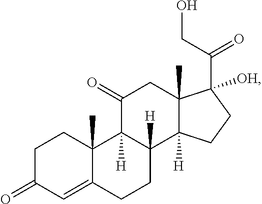

or pharmaceutically acceptable salt thereof.

As described herein, the compositions disclosed herein may comprise a combination of any one or more of the bioactive compounds described herein. Accordingly, the compositions disclosed herein may comprise a combination of any one or more of estradiol, 5α-dihydrotestosterone, estrone, estriol, finasteride, cortisone and spironolactone. In some embodiments, the compositions disclosed herein comprise a combination of any one or more of estradiol, 5α-dihydrotestosterone, estrone, estriol, and finasteride. In some embodiments, the compositions disclosed herein comprise a combination of cortisone and spironolactone.

The concentration of the one or more bioactive compounds will be dependent on the specific nature of the bioactive compounds and/or the final application for the composition.

In one embodiment the composition is formulated so that a concentration of about 10 ng to about 1000 ng of the bioactive compound (for example estradiol) is delivered with a 2×2 cm² application to the skin. The concentration of the bioactive compound (for example estradiol), may be in an amount of about, or at least about: 10 ng, 20 ng, 30 ng, 40 ng, 50 ng, 60 ng, 70 ng, 80 ng, 90 ng, 100 ng, 150 ng, 200 ng, 250 ng, 300 ng, 350 ng, 400 ng, 450 ng, 500 ng, 550 ng, 600 ng, 650 ng, 700 ng, 750 ng, 800 ng, 850 ng, 900 ng, 950 ng, or 1000 ng.

In another embodiment, the composition is formulated such that the concentration of one or more bioactive compounds penetrating a sample of skin, to which the composition is applied is at least about 27 ng/cm². In another embodiment, the composition is formulated such that the concentration of one or more bioactive compounds penetrating a sample of skin, to which the composition is applied is about, at least about: 25 ng/cm², 30 ng/cm², 35 ng/cm², 40 ng/cm², 45 ng/cm², 50 ng/cm², 55 ng/cm², 60 ng/cm², 65 ng/cm², 70 ng/cm², 75 ng/cm², 80 ng/cm², 85 ng/cm², 90 ng/cm², 95 ng/cm², 100 ng/cm², 105 ng/cm², 110 ng/cm², 115 ng/cm², 120 ng/cm², 125 ng/cm², 130 ng/cm², 135 ng/cm², 140 ng/cm², 145 ng/cm², or 150 ng/cm².

In yet another embodiment the composition is formulated such that the concentration of one or more bioactive compounds is provided in a concentration of about, or less than about: 1000 ng/cm², 990 ng/cm², 980 ng/cm², 970 ng/cm², 960 ng/cm², 950 ng/cm², 940 ng/cm², 930 ng/cm², 920 ng/cm², 910 ng/cm², 900 ng/cm², 890 ng/cm², 880 ng/cm², 870 ng/cm², 860 ng/cm², 850 ng/cm², 840 ng/cm², 830 ng/cm², 820 ng/cm², 810 ng/cm², 800 ng/cm², 790 ng/cm², 780 ng/cm², 770 ng/cm², 760 ng/cm², 750 ng/cm², 740 ng/cm², 730 ng/cm², 720 ng/cm², 715 ng/cm², or 710 ng/cm².

In some embodiments, the additional step of testing the serum level of the one or more bioactive compounds of the compositions disclosed herein in the treated subject and determining that the serum level of the one or more of the bioactive compounds of the subject is normal or not substantially elevated compared to the serum level of the one or more bioactive compounds of subjects in the same sex and age group as the treated subject may comprise monitoring the systemic level (e.g., serum level), of the one or more bioactive compounds over time with periodic follow up (e.g., by regular blood or urine tests). For example, regular blood or urine samples can be obtained from a treated subject and assessed for the presence of the bioactive compound as discussed above.

### Penetration Enhancer

The compositions disclosed herein may comprise one or more penetration enhancers, for example, chemical penetration enhancers. Accordingly, in some embodiments, a penetration enhancer is provided for increasing the permeability of skin or mucosal tissue for topical administration of at least one bioactive compound. Ideally, such penetration enhancers (or “permeation enhancers”), are compounds that are innocuous and serve merely to facilitate diffusion of the bioactive compound through the stratum corneum.

In one embodiment, the one or more penetration enhancers are compounds that penetrate into the skin of the subject and interact with skin constituents to promote or increase the flux of one or more bioactive compounds and/or reversibly decrease the barrier resistance of the skin to which a composition as described herein is applied.

In another embodiment, the one or more penetration enhancers in the compositions disclosed herein promote or increases the flux of one or more bioactive compounds and/or reversibly decrease the barrier resistance of the skin to which the composition is applied, in comparison so a similar composition where no penetration enhancer has been added.

In yet another embodiment, the penetration enhancer increases the delivery of one or more bioactive compounds through the skin to which a composition is applied, but does not deliver the one or more bioactive compounds to the bloodstream.

Various compounds for enhancing the permeability of skin are known in the art and may be used in the compositions disclosed herein. Compounds that have been used to enhance skin permeability include, but are not limited to: sulfoxides such as dimethylsulfoxide (DMSO) and decylmethylsulfoxide (C10MSO); ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin (U.S. Pat. No. 4,783,450); the 1-substituted azacycloheptan-2-ones, particularly 1-n-dodecyl-cyclazacycloheptan-2-one (available under the trademark Azone® from Nelson Research & Development Co., Irvine, Calif; see U.S. Pat. Nos. 3,989,816, 4,316,893, 4,405,616 and 4,557,934); alcohols such as ethanol, propanol, octanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol (PG), ethylene glycol, glycerol, butanediol, polyethylene glycol, and polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly salicylic acid and salicylates, citric acid and succinic acid.

One or more penetration enhancers may be selected from, but not limited to: non-polar solvents, polar protic solvents, aprotic polar solvents, alcohols (optionally polyols), ionic surfactants, non-ionic surfactants solvents, aliphatic hydrocarbons, fatty acids, and mixtures thereof.

In an embodiment, the one or more penetration enhancers may include, but are not limited to: water, sulphoxides (e.g., dimethylsulphoxide, DMSO), azones (e.g., laurocapram), pyrrolidones (e.g., 2-pyrrolidone), alcohols and alkanols (e.g., ethanol, or decanol), glycols (e.g., propylene glycol (PG), a common excipient in topically applied dosage forms), surfactants (also common in dosage forms), fatty acids, and terpenes and terpenoids. It will be appreciated that the one or more solvents, as described herein, may also have penetration enhancing properties.

In one embodiment, one or more penetration enhancers may be selected from, but not limited to: ethanol, dimethyl sulfoxide, propylene glycol, isopropanol, polyoxyethylene (20) sorbitan monooleate, urea, sodium docecyl sulfate, menthol, limonene, oleic acid, isopropyl myristate, undecanoic acid, and mixtures thereof. In some embodiments, the one or more penetration enhancers is ethanol, dimethyl sulfoxide, propylene glycol, and/or isopropyl myristate.

The concentration of the one or more penetration enhancers may be dependent on a number of factors including the type of penetration enhancer or penetrations enhancers used, the type of microparticles utilised, and/or the selection of the one or more bioactive compounds.

In one embodiment the penetration enhancer defined herein is used in conjunction with at least one bioactive compound which is estradiol or a derivative thereof.

In one embodiment the penetration enhancer defined herein is used in conjunction with at least one bioactive compound selected from 5α-dihydrotestosterone, estrone, estriol, or cortisone, or a derivative thereof.

In one embodiment the penetration enhancer defined herein is used in conjunction with at least one bioactive compound which is finasteride or a derivative thereof.

In one embodiment the penetration enhancer defined herein is used in conjunction with at least one bioactive compound which is spironolactone or a derivative thereof.

In one embodiment the penetration enhancer defined herein is used in conjunction with at least one bioactive compound which is minoxidil, minoxidil sulphate, or a derivative or a mixture thereof.

In one embodiment the penetration enhancer defined herein is used in conjunction with at least one bioactive compound which is a vitamin selected from vitamin A, B, C, D3, and/or E, or a derivative thereof.

In one embodiment the penetration enhancer defined herein is used in conjunction with at least one bioactive compound which is a retinoid. In certain embodiments, the retinoid is selected from retinoic acid, tretinoin, and derivatives or mixtures thereof

In one embodiment the penetration enhancer comprises ethanol.

In one embodiment the penetration enhancer comprises dimethyl sulfoxide

In one embodiment the penetration enhancer comprises propylene glycol.

In one embodiment the penetration enhancer comprises isopropanol.

In one embodiment the penetration enhancer comprises polyoxyethylene (20) sorbitan monooleate (Tween 80).

In one embodiment the penetration enhancer comprises urea.

In one embodiment the penetration enhancer comprises sodium dodecyl sulfate.

In one embodiment the penetration enhancer comprises menthol.

In one embodiment the penetration enhancer comprises limonene.

In one embodiment the penetration enhancer comprises oleic acid.

In one embodiment the penetration enhancer comprises isopropyl myristate.

In one embodiment the penetration enhancer comprises undecanoic acid.

The one or more penetration enhancers may be present (v/v % or w/v %) in the compositions disclosed herein in a range from about 0.1% to about 30%, for example, present in an amount of about, or at least about: 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the one or more penetration enhancers is present in the composition in an amount of about: 0.1%, 0.2%, 0.3%, 0.4%, 0.5%, 0.6%, 0.7%, 0.8%, 0.9%, 1%, or about 1.1%, 1.2%, 1.3%, 1.4%, 1.5%, 1.6%, 1.7%, 1.8%, 1.9%, 2%. In some embodiments, the one or more penetration enhancers is present in an amount from about 0.01 wt % to about 3 wt %, 4 wt %, or 5 wt % based on total weight of the composition. In some embodiments, the one or more penetration enhancers is present in an amount from about 5 wt % to about: 6 wt %, 7 wt %, 8 wt %, 9 wt %, 10 wt %, 11 wt %, 12 wt %, 13 wt %, 14 wt %, or 15 wt. % based on total weight of the composition. The penetration enhancer can be provided in an amount (as a weight % based on total weight of the composition) of less than about 15, 10, 5, 1, 0.5, 0.1, 0.05, or 0.01. The penetration enhancer can be provided in an amount (as a weight % based on total weight of the composition) of at least about 0.01, 0.05, 0.1, 0.5, 1, 5, 10, or 15. The one or more penetration enhancers can be in a range provided by any two of these upper and/or lower amounts. In some embodiments, the one or more penetration enhancers is present in the compositions disclosed herein.

### Microparticles

As defined above, the compositions disclosed herein comprise a plurality of microparticles.

In the compositions disclosed herein, the microparticles may be composed of any suitable material known in the art. In one embodiment, the microparticles comprise a material having sufficient strength and rigidity to be able to penetrate a biological barrier. Examples of such material include but are not limited to silicon, glass, metal, polymers, ceramics, cellulose derived materials, and mixtures thereof.

In the context of the present disclosure, a “biological barrier” may refer to the skin, the surface of the skin, or a specific part of the skin such as stratum corneum of the skin. Other biological barriers include, for example, mucous membranes such as oral mucosa, vaginal or cervical epithelium or anal canal, as well as scalp, hair and nails.

In some embodiments, the biological barrier is the skin surface. In some embodiments, the biological barrier is the stratum corneum of the skin. In an embodiment, the biological barrier is the stratum corneum of human skin.

In some embodiments, the microparticles in the compositions disclosed herein only penetrate the stratum corneum of the skin. In some embodiments, the microparticles in the compositions disclosed herein only penetrate the stratum corneum and viable epidermis of the skin.

In parts of this disclosure, the microparticles may be referred to as Foroderm.

In some embodiments, the microparticles are composed of a biocompatible material that can be safely applied to the skin, such as silicon, carbohydrates or polymers. In some embodiments, the material is biodegradable. In some embodiments, the material is inert.

As used herein, a “biocompatible” material is one that does not generally cause significant adverse reactions (e.g., toxic or antigenic responses), in the body, whether it degrades within the body, remains for extended periods of time, or is excreted whole. A “biodegradable” material is one that relatively easily degrades under biological conditions.

In one embodiment the microparticles comprise, consist essentially of, or consist of a material selected from, but not limited to: silicon, silica, and mixtures thereof. In another embodiment, the microparticles comprise, consist essentially of, or consist of a material with similar mechanical properties to at least one material selected from: silicon, silica, and mixtures thereof. However, other suitable materials may also be used, such as metals, plastics, (e.g., biocompatible polymers,) cellulose derived materials or ceramic materials.

In one embodiment the microparticles comprise, consist essentially of, or consist of silica. Silica may be conveniently used due biocompatibility, suitable mechanical properties, relative low cost, and availability. Microparticles formed form silica may be advantageously chemically and/or biologically inert.

Various polymeric materials such as poly-l-lactic acid, poly-glycolic acid, poly-carbonate, poly-lactic-co-glycolic acid (PLGA), poly-dimethylsiloxane, a copolymer of methyl vinyl ether and maleic anhydride, cellulose-derived materials such as carboxymethyl cellulose, maltose, dextrin and galactose can all be used to fabricate the microparticles.

In some embodiments, the microparticles comprise a material having sufficient strength and rigidity to withstand an applied force of at least 13 MPa.

In some embodiments, the microparticles are eliminated or dissolved after application. Depending on the composition of the microparticles, the microparticles that penetrate the skin barrier may remain therein for 3-4 weeks and be removed by the natural skin turnover. For example, it has been previously shown that the microparticles made of silica are well tolerated in human patients, with the microparticles eventually sloughing off.

The microparticles can be made to different sizes depending on the application.

In some embodiments, the microparticles are dimensioned to penetrate through a stratum corneum of skin. The dimensions may vary depending on the material of the microparticles.

The microparticles may take any geometric form known in the art. In one embodiment the microparticles are cylindrical, or substantially cylindrical.

In one embodiment: at least a portion of the microparticles, substantially all the microparticles, or all the microparticles are not spherical. In another embodiment: at least a portion of the microparticles, substantially all the microparticles, or all the microparticles are not substantially spherical.

In one embodiment all the microparticles comprise, consist essentially or consist of the same material, for example silicon or silica. In another embodiment, at least a portion, substantially all, or all of the microparticles, are hydrophilic. In another embodiment, at least a portion, substantially all, or all of the microparticles, are hydrophobic. In yet another embodiment a composition described herein comprises, consists essentially of, or consists of, one type of microparticle (e.g., comprising or consisting of the same material). In yet another embodiment a composition described herein comprises, consists essentially of, or consists of, a plurality different types of microparticles (e.g., comprising or consisting of at least two different materials).

In some embodiments, at least a portion of the microparticles comprise a circular, substantially circular, or oval face with a diameter or length in the range of about 5 μm to about 15 μm, for example 6 μm to about 12 μm. The diameter or length may be about, or at least about: 1 μm, 2 μm, 3 μm, 4 μm, 5 μm, 6 μm, 7 μm, 8 μm, 9 μm, 10 μm, 11 μm, 12 μm, 13 μm, 14 μm, or 15 μm. In some embodiments, the microparticles have a diameter in the range of: about 4 μm to about 15 μm, about 5 μm to about 15 μm, about 6 μm to about 14 μm, about 6 μm to about 13 μm, about 6 μm to about 12 μm, about 7 μm to about 11 μm, about 8 μm to about 10 μm. The microparticles may have a diameter of: about 4 μm, about 5 μm, about 6 μm, about 7 μm, about 8 μm, about 9 μm, about 10 μm, about 11 μm, about 12 μm, about 13 μm, about 14 μm, or about 15 μm, for example about 6 μm, about 7 μm, about 8 μm, about 9 μm, about 10 μm, about 11 μm, or about 12 μm.

The microparticles may be elongate.

In some embodiments, at least a portion of the microparticles have a length of about, or at least about: 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, 205 μm, 210 μm, 215 μm, or 220 μm. In some embodiments, at least a portion of the microparticles have a length of about, or at least about 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, or 150 μm.

In some embodiments, the microparticles have a length of up to about: 250 μm, 240 μm, 230 μm, 220 μm, 210 μm, 200 μm, 190 μm, 180 μm, 170 μm, 160 μm, 150 μm, 140 μm, 130 μm, 120 μm, 110 μm, 100 μm, 90 μm, 80 μm, 70 μm, 60 μm, 50 μm, 40 μm, or 30 μm. In some embodiments, the microparticles have a length of up to 220 μm, 210 μm, 200 μm, 190 μm, 180 μm, 170 μm, 160 μm, 150 μm, 140 μm, 130 μm, 120 μm, 110 μm, 100 μm, or 90 μm. In some embodiments, the microparticles have a length of up to 180 μm, 170 μm, 160 μm, 150 μm, 140 μm, 130 μm, 120 μm, 110 μm, 100 μm, or 90 μm. In some embodiments, the microparticles have a length of up to 150 μm, 140 μm, 130 μm, 120 μm, 110 μm, 100 μm, or 90 μm.

In some embodiments, the microparticles have a length in a range of: about 20 μm to about 220 μm, about 30 μm to about 210 μm, about 30 μm to about 200 μm, about 40 μm to about 190 μm, about 50 μm to about 180 μm, about 60 μm to about 170 μm, about 70 μm to about 160 μm, about 80 μm to about 150 μm, about 90 μm to about 140 μm, about 100 μm to about 130 μm, about 110 μm to about 130 μm, or about 110 μm to about 120 μm. In some embodiments, the microparticles have a length in a range of about 30 μm to about 100 μm, about 60 μm to about 120 μm, or about 60 μm to about 170 μm, or about 40 μm to about 90 μm, about 70 μm to about 110 μm, or about 70 μm to about 160 μm, for example about 50 μm to about 80 μm, about 80 μm to about 100 μm, or about 80 μm to about 150 μm. In certain embodiments, the microparticles have a length in a range of about 100 μm to about 130 μm.

In some embodiments, the microparticles have a width or diameter of less than: about 30 μm, about 25 μm, or about 20 μm. In some embodiments, the width or diameter is less than: about 19 μm, about 18 μm, about 17 μm, about 16 μm, or about 15 μm. In some embodiments, the width or diameter of elongate microparticles is: about 15 μm, about 14 μm, about 13 μm, about 12 μm, about 11 μm, about 10 μm, about 9 μm, about 8 μm, about 7 μm, about 6 μm, about 5 μm, about 4 μm, about 3 μm, about 2 μm, about 1 μm; for example: about 13 μm, about 12 μm, about 11 μm, about 10 μm, about 9 μm, about 8 μm, about 7 μm, about 6 μm, or about 5 μm. For some materials, the width may be less than about 12 μm, such as in the range of from about 5 to 10 μm, in the case of silica.

In some embodiments, at least a portion of the microparticles are elongate. For example, at least a portion of the microparticles comprise a length that is greater than a perpendicular cross-section of the microparticle. In some embodiments, at least a portion of the microparticles comprise an aspect ratio of greater than about 1. herein, in some embodiments: at least a portion of the microparticles; substantially all of the microparticles, or all of the microparticles, are elongate. For example, at least or at least about: 5%, 10%, 15%, 20%, 25%, 30%, 35%, 40%, 45%, 50%, 55%, 60%, 65%, 70%, 75%, 80%, 85%, 90%, 95%, of microparticles are elongate.

In some embodiments, the microparticles are elongate with an aspect ratio (e.g., length:width) or about or at least about: 10:1, 9.5:1, 9:1, 8.5:1, 8:1, 7.5:1, 7:1, 6.5:1, 6:1, 5.5:1, 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, or 1.1:1.

In some embodiments, the microparticles are elongate with a high aspect ratio. The aspect ratio (e.g., length:width) may be about or at least about 10:1, such as about at least about: 11:1, 12:1, 13:1, 14:1, or 15:1. In some embodiments the aspect ratio (e.g., length:width) is greater than about 20:1. The upper limit on aspect ratio (e.g., length:width) may be around about 200:1, such as about 150:1. In some embodiments, the maximum aspect ratio (e.g., length:width) is about 100:1. In some embodiments the aspect ratio (e.g., length:width) is from about 5:1 to about 50:1. In some embodiments the aspect ratio (e.g., length:width) is from about 5:1 to about 20:1, for example from about 10:1 to about 20:1. In some embodiments, the microparticles have an aspect ratio (e.g., length:width) of about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, or about 5:1.

In some embodiments, the microparticles have the dimensions of about 6 μm diameter at about 50-80 μm length; about 7 μm diameter at about 50-90 μm length; about 8 μm diameter at about 60-100 μm length; about 8 μm diameter at about 60-100 μm length; about 9 μm diameter at about 70-100 μm length; about 9 μm diameter at about 80-120 μm length; about 9 μm diameter at about 90-130 μm length; about 10 μm diameter at about 80-140 μm length; about 10 μm diameter at about 80-150 μm length; about 11 μm diameter at about 80-150 μm length; or about 12 μm diameter at about 80-150 μm length. In some embodiments, the microparticles have the dimensions of about 5-10 μm diameter at about 100-130 μm length.

The microparticles can be a mixture of the different types described herein.

In an embodiment, the microparticles are substantially uniform in size. At least about 50%, or at least about 70% of the microparticles, may have lengths within about 80% of the median length. In an embodiment, at least about 80% of the microparticles have lengths within about 80% of the median length.

The microparticles may be formed by any process known in the art, such as various microfabrication techniques.

The microparticles may be used directly after synthesis or may undergo one or more processes to change one or more of the following characteristics: the topography, the structure, the content, a coating, and mixtures thereof.

The microparticles may be manufactured by milling and/or by cutting.

The microparticles may be prepared using chemical isotropic etching, injection moulding, reactive ion etching, surface/bulk micromachining, micromoulding and lithography-electroforming-replication.

For example, the microparticles may be produced by a fabrication process including the steps of lithography (patterning the design onto the substrates), using deep reactive-ion etching (DRIE) to fabricate pillars and inspecting the etching results and using piranha solution to remove residue.

The microparticles may be processed, for example etched, by a process known in the art. The process may be chemical, for example utilising a chemical process such as with a compound such as sodium hydroxide and/or piranha solution, or using physical means such as reactive ion etching.

The microparticles may be coated with one or more materials. The materials may be crosslinked, or the material may not be crosslinked. The one or more materials may form a dry coating. The one or more materials may be selected from, but not limited to: alginate, cross-linked alginate, chitosan, sugars, or a cellulose such as carboxymethyl cellulose, and mixtures thereof. In some embodiments, the microparticles are coated with a coating material selected from alginate, cross-linked alginate, or carboxymethyl cellulose, or a mixture thereof.

In some embodiments, the microparticles are coated. In some embodiments, the coating comprises the at least one bioactive compound. In some embodiments, the microparticles and the at least one bioactive compound are formulated as a mixture.

In some embodiments, the microparticles are admixed with the at least one bioactive prior to topical application to a body surface (e.g., skin), as a single formulation. The formulation may be formed in advance and stored, or formed just prior to the application. Advantageously, the microparticles can be admixed with an existing or known formulation of the at least one bioactive compound.

In some embodiments, the at least one bioactive compound is formulated to be applied to a body surface (e.g., the skin), separately from the microparticles, such as after the application of the microparticles. For example, the at least one bioactive compound may be in the form of an existing or known formulation of the at least one bioactive compound.

The microparticles may be hollow, substantially hollow, solid, substantially solid, or a mixture thereof.

At least a portion of the microparticles may have one or more flat, or substantially flat ends. In an embodiment at least some of the microparticles have a tapered end geometry. The microparticles may have an end angled at less than about 180° (e.g. less than about: 170°, 160°, 150°, 140°, 130°, 120°, 110°, 100°, 90°, 80°, 70°, 60°, 50°, 40°, or 30°), and may be less than about 200 such as less than about 10°.

At least a portion of the microparticles may have one or more convex ends.

The microparticles may be uniform in size, or may be a range of sizes.

The microparticles may be used in a concentration known to a person skilled in the art. The final concentration may be dependent on a number of factors, including the selection for the one or more bioactive compounds, the one or more penetration enhancers, the specific microparticles selected and/or the final application for the composition comprising the microparticles.

In one embodiment less than about: 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, or 90 wt %, of at least one bioactive compound is present within at least a portion, substantially all, or all of the microparticles. In yet another embodiment, about or at least about: 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, or 90 wt %, of at least one bioactive compound is present on at least one external surface of at least a portion, substantially all, or all of the microparticles (e.g. as one or more coatings).

For the compositions described herein, a single coating may be present on at least a portion of microparticles. In some embodiments multiple coatings may be present on at least a portion of microparticles. For example at least a portion of microparticles comprise a plurality of coating layers. In another embodiment microparticles with different coatings may be mixed together.

In some embodiments, the microparticles are present in the composition disclosed herein in a concentration of about or at least about: 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg, per ml of the composition. In some embodiments, the microparticles are present in the composition disclosed herein at a concentration of up to about 150 mg, 140 mg, 130 mg, 120 mg, 110 mg, 100 mg, 90 mg, 80 mg, 70 mg, 60 mg, 60 mg, 50 mg, 40 mg, 30 mg, 20 mg, 10 mg, 9 mg, 8 mg, 7 mg, 6 mg, 5 mg, 4 mg, 3 mg, 2 mg, or 1 mg per ml of the composition. In some embodiments, the concentration of the microparticles is about 150 mg, 145 mg, 140 mg, 135 mg, 130 mg, 125 mg, 120 mg, 115 mg, 110 mg, 105 mg, 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, or 25 mg per ml of the composition, for example about 150 mg to about 50 mg, 140 mg to about 60 mg, 130 mg to about 70 mg, 120 mg to about 80 mg, 110 mg to about 90 mg, or about 100 mg per ml of the composition. In some embodiments, the microparticles are present in the composition in a concentration of about or at least about 50 mg to about 150 mg per ml, optionally about 70 mg to about 100 mg, per ml of the composition.

In some embodiments, the microparticles may be applied on a body surface area, such as skin, with an area of about, or at least about: 1 cm², 2 cm², 3 cm², 4 cm², 5 cm², 6 cm², 7 cm², 8 cm², 9 cm², 10 cm², 11 cm², 12 cm², 13 cm², 14 cm², 15 cm², 16 cm², 17 cm², 18 cm², 19 cm², 20 cm², 21 cm², 22 cm², 23 cm², 24 cm², 25 cm², 26 cm², 27 cm², 28 cm², 29 cm², 30 cm², 31 cm², 32 cm², 33 cm², 34 cm², 35 cm², 36 cm², 37 cm², 38 cm², 39 cm², 40 cm². In some embodiments, the surface area is about 1 to about 15 cm², about 5 to about 20 cm², about 10 to about 25 cm², about 15 to about 30 cm², about 20 to about 35 cm², about 25 to about 40 cm², about 30 to about 45 cm², about 35 to about 50 cm², about 40 to about 55 cm², about 45 to about 60 cm², about 50 to about 65 cm², about 55 to about 75 cm², about 60 to about 80 cm², about 65 to about 85 cm², about 70 to about 90 cm², about 75 to about 95 cm², about 80 to about 100 cm², about 85 to about 105 cm², about 90 to about 110 cm², about 95 to about 115 cm², about 100 to about 120 cm², about 110 to about 130 cm², about 120 to about 140 cm², about 130 to about 150 cm². In some embodiments, the surface area is about 100 to about 150 cm², about 120 to about 180 cm², about 140 to about 200 cm², about 150 to about 220 cm², about 160 to about 240 cm², about 180 to about 260 cm², about 200 to about 300 cm², or about 200 to about 400 cm². In some embodiments, the microparticles are applied to cover all or substantially all of an area on a body surface, approximately 95%, 90%, 85%, 80%, 75%, 70%, 65%, 60%, 55%, 50%, 45%, 40%, 35%, 30%, 25%, 20%, 15%, 10%, 5%, 4%, 3%, 2% or 1% of the area on the body surface. In some embodiments, the surface area to which the composition is applied is less than 500 cm². For example, the surface area is less than 300 cm², 200 cm², 100 cm², 50 cm², 40 cm², 30 cm², 20 cm², 10 cm², and 5 cm².

In yet another embodiment, about, or at least about: 1 mg, 2 mg, 3 mg, 4 mg, or 5 mg of microparticles are mixed with an amount of liquid of about, or at least about 5 L, 10 μL, 15 μL, 20 μL, 25 μL, 30 μL, 35 μL, 40 μL, 45 μL, 50 μL, 55 μL, 60 μL, 70 L, 80 μL, 90 μL, or 100 μL of liquid per cm² of a body surface (e.g., skin), for applying to at least a portion of the surface.

In some embodiments, the amount of microparticles is at least about: 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg per cm² of a body surface area (e.g., skin).

### Applicator

The compositions disclosed herein can be administered, for example, by applying a force to the composition on a biological barrier such as the surface of the skin so that at least a fraction of the composition penetrates the biological barrier, to facilitate a local delivery of the one or more bioactive compound. For example, the compositions disclosed herein or fractions thereof may only penetrate the stratum corneum of the skin, or the stratum corneum and viable epidermis of the skin, to facilitate a local delivery of the one or more bioactive compound. In some embodiments, the compositions disclosed herein or fractions thereof only penetrate the stratum corneum of the skin to facilitate a local delivery of the one or more bioactive compound. In some embodiments, the compositions disclosed herein or fractions thereof only penetrate the stratum corneum of the skin and viable epidermis of the skin to facilitate a local delivery of the one or more bioactive compound.

The force may be applied by hand or by using an applicator. In some embodiments, the compositions disclosed herein are administered by rubbing or massaging the compositions onto a biological barrier such as the skin by means of the hand or an applicator. In some embodiments, the compositions disclosed herein are rubbed or massaged onto the biological barrier such as the skin by means of an applicator.

Accordingly, in another aspect of the present disclosure, there is provided an applicator adapted to administer (or capable of administering), the compositions disclosed herein. Herein, the applicator may be adapted for applying the compositions disclosed herein to a biological barrier so that at least a fraction of the composition penetrates the biological barrier.

In some embodiments, the applicator is a textured applicator having at least one textured surface. The textured surface may be configured to engage and orientate the microparticles in the compositions disclosed herein at an angle to facilitate penetration of a biological barrier such as the skin surface. In some embodiments, the microparticles in the compositions disclosed herein only penetrate the stratum corneum of the skin. In some embodiments, the microparticles in the compositions disclosed herein only penetrate the stratum corneum and viable epidermis of the skin.

In some embodiments, the applicator is a microtextured applicator having at least one microtextured surface. The microtextured surface may be configured to engage and orientate the microparticles in the compositions disclosed herein at an angle to facilitate penetration of a biological barrier such as the skin surface. In some embodiments, the microparticles in the compositions disclosed herein only penetrate the stratum corneum of the skin. In some embodiments, the microparticles in the compositions disclosed herein only penetrate the stratum corneum and viable epidermis of the skin.

The microtextured applicator can be of various sizes suitable for desired surface areas, and made of different suitable materials, such as biocompatible and non-allergenic material, and may be manufactured, for example, using 3D printing, photolithography or deep reactive ion etching.

In some embodiments, the applicator is an applicator described in patent application WO 2014/094067 A1, the entire contents of which are incorporated herein by reference.

For example, in some embodiments, the applicator includes a microtextured surface, which may be configured to engage and orientate the microparticles of the compositions disclosed herein at an angle to facilitate penetration of the biological barrier. Using the applicator including a microtextured surface, the compositions disclosed herein can be applied to a biological barrier so that at least a fraction of the composition penetrates the biological barrier, including the step of using the applicator to engage and orientate the microparticles of the composition at an angle to facilitate penetration of the biological barrier.

The applicator is an implement for use in applying the compositions disclosed herein, but may is not intended to be a permanent support for the compositions disclosed herein. The compositions disclosed herein are provided separately from the applicator. Some of the microparticles of the compositions disclosed herein may temporarily adhere to the microtextured surface of the applicator, for example the applicator head during and following use. However, generally, after delivery of the compositions, the applicator is removed.

In some embodiments, the microtextured surface of the applicator is treated with the compositions disclosed herein (such as by being coated with the compositions), intended to be delivered through a biological barrier such as the skin surface. In such instances, the compositions disclosed herein can be simultaneously applied to the biological barrier and at least partially delivered through the biological barrier with the aid of the applicator.

In some embodiments, the microparticles of the compositions disclosed herein are applied to the skin in a first step in the absence of the one or more bioactive compound of the compositions disclosed herein. The microparticles of the compositions disclosed herein are thought to form microchannels in the skin as they penetrate the skin under the manual force applied by the applicator surface. In a subsequent step, the one or more bioactive compound of the compositions disclosed herein can be applied to the skin (with or without massaging or rubbing of the bioactive compound), forming the composition disclosed herein in situ; and at least some of the bioactive compound can penetrate through the microchannels formed in the skin thereby delivering the bioactive compound.

The applicator can be held in the hand of an operator and the microtextured surface can be manually massaged or rubbed over the area of skin to which the compositions disclosed herein should be applied. The manual massaging or rubbing can facilitate at least some of the compositions disclosed herein to penetrate the skin.

In an embodiment, the applicator includes a body and an applicator head on which is provided the microtextured surface. In another embodiment, the applicator comprises a glove or a finger cot including the microtextured surface on a fingertip region thereof.

In another embodiment, the applicator includes microtexturing on both the head and body thereof. For example, the applicator may be substantially cylindrical in shape. The cylindrical applicator can have a body in the form of a shaft and a head in the form of a tip of the shaft. The cylinder can have a microtextured surface on the shaft and the tip. In one embodiment, the cylindrical applicator may be a rod or a baton. This embodiment is suitable for use where the biological barrier is provided within a body cavity and the applicator is inserted into the body cavity during use. At least the portion of the shaft intended for insertion can comprise a microtextured surface. The body cavity can be, for example, a vaginal tract.

The applicator is intended for manual use; however, there may be mechanical and/or electronic integration within the applicator. For example, there could be mechanical agitation of the microtextured surface of the applicator. The agitation can be vibration and/or oscillation. The applicator could include a sonophoresis or electrophoresis probe or something similar that provides a ‘non-invasive’ force to enhance delivery. Any agitation may work in conjunction with the manual massaging or rubbing application technique.

The hand-held applicator may further comprise a force controller. The force controller enables the user to vary the force with which the applicator is manually applied to the skin such that an optimal force for penetration of the skin may be achieved. The force controller may also include an applied force meter which indicates to the user when the optimal manual force is achieved.

The force applied by the applicator surface to the compositions disclosed herein is a force within a range that can be applied manually by a human operator. In an embodiment, the force may be greater than 0.01 Newtons, such as a minimum of 0.1 Newtons. In an embodiment, the applied force is at least 0.2 Newtons. In one embodiment, the applied force is 2.5 Newtons. The maximum force may be 10 Newtons.

The compositions disclosed herein may show enhanced penetration of a biological barriers such as the stratum corneum of the skin when they are applied using the applicator than when applied manually without an applicator, e.g., by rubbing with a finger. For example, intradermal delivery of the compositions disclosed herein may be improved when the compositions are applied using the applicator.

As used herein, the term “microtextured” means that the surface has raised and/or lowered surface features on the micrometres to millimetre scale.

In an embodiment, the textured surface includes angled faces that define spaces for receiving the microparticles of the compositions disclosed herein during use of the applicator. The angled faces may be flat or curved. In use of the applicator of this embodiment, at least an end region of each microparticle of the compositions disclosed herein is received in a space such that at least one angled face engages the end of the microparticle and defines the orientation of the microparticle. The spaces may be each defined by one or more angled faces.

In one embodiment, the spaces may be defined between protrusions on the textured surface. The protrusions may comprise a series of laterally spaced elongate or discrete microprotrusions or microridges wherein each protrusion or ridge provides two or more generally inwardly angled faces. A first face can engage the end of a microparticle of the compositions disclosed herein and an adjacent second face can define the orientation of the microparticle. There can be bases or valleys formed between the ridges.

In another embodiment, the spaces may be defined by recesses in the textured surface. The recesses may comprise a series of laterally spaced elongate or discrete microrecesses. The microrecesses may be a series of dimples. In one embodiment, each recess provides two or more inwardly angled faces. One of the faces can engage the end of a microparticle of the compositions disclosed herein and the other can define the orientation of the microparticle. The microtexturing geometry is adapted such that the microparticles cannot be received into and trapped in the texturing e.g., spaces between the faces.

The face that defines the orientation of a microparticle of the compositions disclosed herein may be angled and dimensioned to provide support for at least an end section of the microparticle. In this embodiment, at least the end section of the microparticle may extend across the width of the face. The width of the face may therefore be less than the minimum length of the microparticles of the compositions disclosed herein so that each microparticle can extend beyond the face and penetrate the biological barrier during use of the applicator. The width of this face may be a minimum of 8 microns. The maximum width of this face may be 1 mm.

There may be some geometries of microtexturing that are optimal for engaging and orienting individual microparticles of the compositions disclosed herein and other geometries of microtexturing that are optimal for engaging aggregates of the microparticles of the compositions disclosed herein. The types of microtexturing on the surface of the applicator may differ over the surface. The types of microtexturing may differ to provide optimal penetration of single particles and aggregates of particles. Single particles may be best orientated by relatively small features in the microtexturing. In one embodiment, a peripheral circumferential edge of a generally cylindrical applicator surface may have a first geometry of microtexturing and top surface of the cylinder may have a different geometry of microtexturing.

There may be smooth areas on the surface of the applicator which do not comprise microtexturing. The microtexturing on the surface of the applicator can be in a range of from about 1 to 100%. In an embodiment, at least about 75%, 77%, 80%, 85%, 90% or 95% of the applicator surface comprises microtexturing, with the areas between the microtexturing being substantially smooth. In an embodiment, the surface of the applicator comprises 100% microtexturing with no smooth areas. Different amounts of microtexturing can deliver different doses of a compound. In one embodiment, at a given concentration of a compound in a composition or formulation, different amounts of microtexturing can be selected to control dose delivery. For example, 95% microtexturing of the surface of the applicator may provide 95% of the available dose of a compound of the composition; while 50% of microtexturing on the surface of the applicator may provide 50% of the available dose of a compound in the composition.

It is hypothesised that following application of the compositions disclosed herein there is negligible disruption to the skin due to the micro-sized diameter of the microparticles of the compositions disclosed herein and the skin's elastic properties. It is believed that the microchannels formed by the microparticles of the compositions disclosed herein close following their penetration. This attribute of quick closure of pathways created by the microparticles of the compositions disclosed herein following application is beneficial when considering potential foreign substance (microbial) infiltration.

At least that part of the applicator comprising the textured surface is constructed from material that is harder than skin as otherwise the microparticles of the compositions disclosed herein could embed in the applicator rather than in the skin.

The microtexturing on the surface of the applicator can engage and align the microparticles of the compositions disclosed herein. It is thought that the bases or valleys within the textured surface are the most common areas that provide resistance to the microparticles of the compositions disclosed herein when pushing them into the skin.

In some embodiments, the material of the applicator surface can be chosen or adapted to be attractive to the microparticles of the compositions disclosed herein in order to engage and orient them. For example, the material of the applicator may differ in any bases, valleys or depressions within the microtexturing to provide attraction in those areas. The attraction can be provided in the form of adhesive and/or magnetic materials. If portions of the microtexturing are attractive, it may be possible to attract the microparticles to the applicator head and have then aligned by e.g., the ridges in the texturing.

In an embodiment, the material from which the microtexturing is formed is soft and/or malleable. A soft material may allow for deeper penetration of the compositions disclosed herein. If the material from which the microtexturing is formed is too hard, some of the microparticles of the compositions disclosed herein may be broken e.g., by snapping, which is undesirable. The material may comprise a UV curable polymer.

The microtextured surface of the applicator can be formed by any method that results in surface microtexturing. The method can be 3D printing, photolithography or deep reactive ion etching.

In some embodiments, it is advantageous if the microparticles of the compositions disclosed herein penetrate the biological barrier at an angle. The angle can be sufficiently oblique to allow the microparticles to penetrate the stratum corneum, but not so deep that the microparticle penetrates the dermis. Without being bound by theory, the significance of an angled penetration profile may also be that the microparticles of the compositions disclosed herein can create microchannels with a greater surface area for delivery of the one or more bioactive compound of the compositions disclosed herein when compared to perpendicular or 90 degree penetration (relative to the skin). This can mean that the delivery methods described herein are advantageous over prior art such as microneedles and gas jet injectors. It is hypothesised that an angled penetration of the microparticles of the compositions disclosed herein can disrupt a greater region of viable epidermis, increasing its permeability yet being minimally invasive by minimising damage to the dermal-epidermal junction, dermal capillaries and pain receptors.

The angled faces on the microtextured surface may be orientated such that during use of the applicator, the microparticles of the compositions disclosed herein may be orientated at an acute angle with respect to the surface of a biological barrier such as the skin to facilitate penetration of the biological barrier. In an embodiment, the microparticles of the compositions disclosed herein may be orientated, such as to penetrate e.g., the skin at an angle of greater than 0 and less than about 45 degrees, such as in the range of from about 5 to about 30 degrees, for example in the range of from about 7 to about 25 degrees. In some embodiments, it is expected that the angle of penetration will rarely be greater than 45 degrees. The angle could be greater than 45 degrees if the microparticles of the compositions disclosed herein are clumped, or if the skin is deformed around the applicator. For example, skin with degraded collagen and elastin (e.g., aged skin) could potentially deform around the applicator and during massaging the microparticles could penetrate at close to perpendicular (90 degrees).

The textured surface may have a generally convex, concave, rectangular, v-shaped or other profile during use. In one embodiment, the microtextured surface is generally convex. Experimentation with different applicator designs has found that a generally convex textured surface may better align the microparticles of the compositions disclosed herein into an orientation that allows them to penetrate the skin than either, for example, a smooth convex surface or a concave textured surface, which can result in poor penetration. Due to the elastic nature of skin, it stretches and moves when force is applied to it. It is therefore believed that a generally convex profile allows optimal contact between the textured surface and the skin when the applicator is manually pressed against the skin. In one embodiment, the portion of the applicator comprising the microtextured surface may be made from a deformable material that can start as concave and become convex upon the application of force.

The applicator surface can cause penetration of from about 1 to about 100 microparticles of the compositions disclosed herein per mm² of a biological barrier such as the skin surface. In some embodiments, the applicator surface can cause penetration of about 40, about 50, about 60, about 70 or about 80 microparticles per mm².

The applicator can cause penetration of the microparticles of the compositions disclosed herein to a depth of at least 40 μm, such as up to 50 μm, for example up to 60 μm. The microparticles of the compositions disclosed herein may not penetrate deeper than 60 μm after application to the skin. Without being bound by theory, this may be due to the dermal collagen acting as a physical penetration barrier to the microparticles.

It is proposed that with the aid of the applicator, an average of up to about 15%, such as between 10 to 15% of the microparticles of the compositions disclosed herein provided to the skin may penetrate into the dermis.

The number of the microparticles of the compositions disclosed herein in the body will decrease over time as they are expelled from the body. In some embodiments, it is expected that the microparticles of the compositions disclosed herein may be completely expelled from the body after about 1, 2 or 3 weeks. The potential mechanism of microparticle elimination could be associated with natural skin turn-over (desquamation). In humans, over approximately three weeks, new skin cells (keratinocytes) are generated within the stratum basale. The keratinocytes migrate upwards forming the cellular viable epidermis. As the cells migrate closer to the surface of the skin they begin to terminally differentiate (lose their cell nuclei) and flatten. This process renews the stratum corneum and aids in the natural removal of foreign bodies within the skin.

The applicator can be provided as part of a kit that comprises the compositions disclosed herein. Thus, in a further aspect of the present disclosure, there is provided a kit comprising the applicator described herein and the compositions disclosed herein. In some embodiments, at least a portion of the applicator surface is coated with the composition described herein. The kit can be provided together with instructions for use.

### Methods of Treatment

In certain aspects of the disclosure, the compositions disclosed herein are administered in a method of treatment or prophylaxis. As used herein, the terms “treating”, “treat” or “treatment” include administering a composition of the disclosure to thereby reduce or eliminate at least one symptom of the condition to be treated. In an example, treatment includes administering a composition disclosed herein to the skin of a subject. In an example, treatment response is determined relative to a baseline measure prior to treatment. In another example, treatment response is determined relative to a control patient population. The term “prophylaxis”, “prophylactic treatment”, “prophylactic”, “preventative”, or “preventing”, as used herein, refers to treating a subject who does not yet exhibit symptoms of a disease or condition, but who is susceptible to, or otherwise at risk of, a particular disease or condition, whereby the treatment reduces the likelihood that the patient will develop the disease or condition. In some embodiments, the compositions disclosed herein are administered to cure, ameliorate, slow progression of, prevent, or reduce the likelihood of onset of the identified diseases or conditions, or to exhibit a detectable therapeutic or prophylactic effect.

In an example, the one or more bioactive compounds of the compositions disclosed herein are administered in combination with a penetration enhancer. Appropriate penetration enhancers will be apparent to those of skill in the art based on the bioactive compound and, the condition being treated. In an embodiment, a combination of penetration enhancers may be used in conjunction with one or more bioactive compounds of the compositions disclosed herein.

The compositions disclosed herein are particularly advantageous for topical delivery of bioactive compounds. Accordingly, the present disclosure also provides methods of treatment that include administering to a subject the composition disclosed herein, the pharmaceutical composition disclosed herein, or the cosmeceutical composition disclosed herein.

Without being bound by theory, it is proposed that the microparticles in the compositions disclosed herein preferentially penetrate the outer parts of a biological barrier such as the skin, for example the stratum corneum of the skin or the stratum corneum and viable epidermis of the skin, allowing for localised absorption of a bioactive compound, such as localised intradermal absorption. For example, the absorption may involve partitioning of the bioactive compound from the applied composition into the stratum corneum and the epidermis. In this way, the compositions of the present disclosure can achieve, for example, topical delivery of a bioactive compound across the stratum corneum and retention of the majority of the bioactive compound intracutaneously such that it does not enter the bloodstream in significant amounts. It was realised by the present inventors that such enhanced topical localised delivery is particularly suited to, for example, the topical administration of bioactive compounds that act on the nuclear receptor superfamily, such as steroids, thyroid hormones, retinoids, vitamins, and the treatment of associated conditions. In particular, the methods disclosed herein are suitable for topical delivery of steroid hormones and compounds that act on steroid hormone regulation. In some embodiments, the methods disclosed herein are particularly suitable for topical delivery of one or more bioactive compounds selected from a steroid or a corticosteroid, optionally selected from the group consisting of: estradiol, estrone, estriol, 5α-dihydrotestosterone, finasteride, cortisone, spironolactone, or a mixture thereof, minoxidil, minoxidil sulphate, or a derivative or a mixture thereof, a vitamin, optionally selected from vitamin A, B, C, D3, and/or E; or a retinoid, optionally selected from the group consisting of: retinoic acid, tretinoin, and mixtures thereof. Other bioactive compounds may include: 5-aminolevulinic acid hydrochloride, diclofenac sodium, sodium fluorescein, or mixtures thereof.

In some embodiments, the compositions and methods of treatment described herein result in limited or no systemic absorption or accumulation of one or more of the administered bioactive compounds. In other words, in certain examples, the bioactive compound is localised to the intracutaneous regions of a subject treated according to the methods disclosed herein. In such embodiments, it may be necessary to monitor the blood of a subject periodically to confirm that the bioactive compound has not entered the bloodstream in significant amounts. For example, regular blood samples can be obtained from a treated subject and assessed for the presence of the bioactive compound by methods that are known and readily available to those skilled in the art.

Accordingly, in some embodiments, the methods of the present disclosure further include, after the administration step, an additional step of testing the presence or level of the one or more bioactive compounds of the compositions disclosed herein in the treated subject. In some embodiments, the additional step comprises testing the serum level of the one or more bioactive compounds in the subject.

The additional step may comprise determining the concentration of the one or more bioactive compounds in a sample from the subject, optionally wherein the sample is isolated from the subject. In some embodiments, the sample is a urine sample, blood sample, serum sample or plasma sample.

As will be apparent to those skilled in the art, endogenous bioactive compounds may be present in the body of a treated subject at a certain normal (e.g., baseline), level for subjects of the same sex and/or age group. In the context of the present disclosure, “endogenous” compounds refers to compounds that originate within the body, such as hormones. In such examples, it may not feasible to rely on the mere presence of a bioactive compound as an indicator of whether the bioactive compound has entered a treated subject's bloodstream after administering a composition disclosed herein. In such examples, a treated subject's level (e.g., serum level), of the bioactive compound may be compared to a control. In an example, the control may be a treated subject's level (e.g., serum level), of the bioactive compound prior to treatment. In another example, the control is the level (e.g., serum level), of the bioactive compound observed in untreated subjects of the same sex. In certain examples, the control is the level (e.g., serum level), of the bioactive compound observed in untreated subjects of the same sex and age. Typically, control levels of endogenous bioactive compounds in untreated subjects will be readily available to those skilled in the art from the literature.

As used herein, a “normal” level of a bioactive compound is a level (e.g., serum level), of the bioactive compound which is relatively close to the average level of the bioactive compound for subjects of the same sex and/or age group. For example, in some embodiments, the methods disclosed herein may also include, after the administration step, the additional step of testing the serum estradiol level of a treated female subject of a certain age group and determining that the serum estradiol level of the treated female subject is normal for women in the same age group as the treated female subject. In some embodiments, the female subject is a postmenopausal female subject.

It will also be apparent to those skilled in the art that exogenous bioactive compounds may not be normally present in the body of a treated subject in substantial amounts prior to administration of such exogenous bioactive compounds. In the context of the present disclosure, “exogenous” compounds are compounds that do not originate within the body, such as small molecule drugs. The baseline level of exogenous bioactive compounds of the treated subject may be about zero, in which cases the mere presence of a bioactive compound is an indicator of whether the bioactive compound has entered a treated subject's bloodstream after administering a composition disclosed herein. In other examples, the subject may have undergone previous therapy with the exogenous bioactive compound. In such examples, a treated subject's level (e.g., serum level), of the bioactive compound may be compared to a control, wherein the control is the level (e.g., serum level), of the bioactive compound observed in the subject prior to administering treatment.

For example, in some embodiments, the methods disclosed herein may also include, after the administration step, the additional step of testing the serum level of minoxidil or a derivative thereof, minoxidil sulphate or a derivative thereof, finasteride or a derivative thereof, or a mixture thereof, of a treated male subject and determining that the serum level of minoxidil or a derivative thereof, minoxidil sulphate or a derivative thereof, finasteride or a derivative thereof, or a mixture thereof, of the treated male subject is not substantially elevated compared to a control serum level of minoxidil sulphate or a derivative thereof, finasteride or a derivative thereof, or a mixture thereof, in the subject.

In one embodiment, provided herein is a method of treating and/or preventing at least one menopause symptom, the method comprising administering the composition disclosed herein, the pharmaceutical composition disclosed herein, or the cosmeceutical composition disclosed herein. In some embodiments, the at least one bioactive compound in the composition disclosed herein comprises estradiol, estrone, estriol or a retinoid, or a derivative thereof. In some embodiments, the at least one bioactive compound in the composition disclosed herein comprises estradiol or a derivative thereof. Herein, at least one menopause symptom that may be alleviated by the compositions or methods disclosed herein may be selected from, but not limited to: hot flushes, night sweats, crawling or itching sensations under the skin, headache, tiredness, urinary frequency, vaginal dryness, and mixtures thereof. In some embodiments, the symptoms of menopause that can be alleviated by the compositions or methods disclosed herein include the loss of skin thickness and elasticity. In some embodiments, the compositions and methods disclosed herein are useful to increase epidermal hydration, skin elasticity, skin thickness, and/or reduce skin wrinkles in menopausal subjects. Furthermore, in some embodiments, the compositions and methods disclosed herein are useful to enhance the content and quality of collagen and the level of vascularisation.

In another embodiment, provided herein is a method of hydrating and/or increasing collagen formation in at least a portion of skin, the method comprising administering the composition disclosed herein, the pharmaceutical composition disclosed herein, or the cosmeceutical composition disclosed herein. In some embodiments, the at least one bioactive compound in the composition disclosed herein comprises estradiol, estrone, estriol or a retinoid, or a derivative thereof. In some embodiments, the at least one bioactive compound in the compositions disclosed herein comprises estradiol or a derivative thereof. The methods described herein involving topical administration are applicable to, for example, maintaining skin condition and/or for stimulating/increasing collagen formation, for rejuvenating skin, increasing the skin thickness and/or elasticity. The methods can alleviate the changes due to aging that occur in human skin, such as development of fine lines (wrinkles), loss of elasticity (sagging), as well as changes in skin tone, texture, thickness, and moisture content.

In another embodiment, provided herein is a method of treating and/or preventing hair loss, the method comprising administering the composition disclosed herein, the pharmaceutical composition disclosed herein, or the cosmeceutical composition disclosed herein, wherein the composition is formulated for topical application. In an embodiment, the at least one bioactive compound in the composition disclosed herein comprises minoxidil or a derivative thereof. In one embodiment, the at least one bioactive compound comprises minoxidil sulphate, or a derivative thereof. In an embodiment, the at least one bioactive compound comprises finasteride or a derivative thereof. In an embodiment, the method relates to the treatment of a male subject. In an embodiment, the subject's hair loss is self-diagnosed. In an embodiment, the hair loss is diagnosed by a treating clinician. In an embodiment, the treatment is administered to the head of the subject. In some embodiments, the hair loss is male balding. In one embodiment, the hair loss is male pattern balding. In an embodiment, the hair loss is treated in a female subject. In some embodiments, the hair loss is caused by androgenetic alopecia (AGA). In an embodiment, the treatment is prophylactic.

In another embodiment, provided herein is a method of treating and/or preventing hirsutism (excessive hair growth), such as excessive hair growth in non-scalp regions of the body, the method comprising administering the composition disclosed herein, the pharmaceutical composition disclosed herein, or the cosmeceutical composition disclosed herein, wherein the composition is formulated for topical application. In some embodiments, the at least one bioactive compound in the compositions disclosed herein comprises spironolactone or a derivative thereof. In an embodiment, the method relates to the treatment of a female subject. The excessive hair may be on a non-scalp region of a subject, for example, on the face, chest and/or back. In an embodiment, the method relates to the treatment of a male subject.

In another embodiment, provided herein is a method of treating and/or preventing a dermatological condition or disease, the method comprising administering the composition disclosed herein, the pharmaceutical composition disclosed herein, or the cosmeceutical composition disclosed herein, wherein the composition is formulated for topical application. Examples of dermatological conditions or diseases suitable for treatment by the methods described herein include, but are not limited to, skin aging conditions, epicondylitis, psoriasis, and dermatitis. The compositions disclosed herein are also suitable for the treatment and/or prevention of formication.

In some embodiments, the dermatological condition or disease is acne. As used herein, the term “acne” refers to a skin condition that occurs when hair follicles become plugged with oil and/or dead skin cells. The condition generally causes whiteheads, blackheads or pimples on a subject's skin. In an embodiment, the acne is common acne. In another example, the acne is cystic acne. Other types of acne include acne with papules, pustules or nodules, acne mechanica and acne conglobate. In an embodiment, the compositions disclosed herein are applied directly to a zone of acne breakout. In another example, the compositions are applied prophylactically in the absence of acne. In an example, the composition is applied to a subject who has previously presented with acne or symptoms thereof.

In some embodiments, the at least one bioactive compound in the composition disclosed herein, the pharmaceutical composition disclosed herein, or the cosmeceutical composition disclosed herein suitable for the treatment of acne comprises a vitamin A derivative. In an embodiment, the vitamin A derivative is retinol or retinoic acid. Other examples of suitable bioactive compounds include retinyl acetate, retinyl linoleate, retinyl palmitate and retinyl proprionate. In an embodiment, the at least one bioactive compound is tretinoin. In other examples, the at least one bioactive compound is a topical retinoid, adapalene, salicylic acid, azelaic acid and alpha hydroxyacids, and/or topical antibiotic and/or benzoyl peroxide. In an embodiment, the at least one bioactive compound is a steroid. In another embodiment, the at least one bioactive compound is a tetracycline. In another embodiment, the at least one bioactive compound is spironolactone.

Examples of topical anti-acne bioactive compounds suitable for the methods disclosed herein further include adapalene, azelaic acid, benzoyl peroxide, clindamycin and clindamycin phosphate, doxycycline, erythromycin, keratolytics such as salicylic acid and retinoic acid (e.g., “Retin-A”), norgestimate, organic peroxides, retinoids such as isotretinoin and tretinoin, sulfacetamide sodium, and tazarotene. Particular anti-acne agents include adapalene, azelaic acid, benzoyl peroxide, clindamycin (e.g., clindamycin phosphate), doxycycline (e.g., doxycycline monohydrate), erythromycin, isotretinoin, norgestimate, sulfacetamide sodium, tazarotene, etretinate and acetretin.

In another embodiment, provided herein is a method of improving or treating the appearance of skin on a subject, the method comprising administering the composition disclosed herein, the pharmaceutical composition disclosed herein, or the cosmeceutical composition disclosed herein. The compositions disclosed herein are suitable for addressing cosmetic and subjective needs, such as, for example, altering or improving the appearance of the portion of skin; improving skin elasticity, skin hydration, firmness, skin tone, skin colour and/or texture; increasing softness of skin.

In an embodiment, the composition disclosed herein, the pharmaceutical composition disclosed herein, or the cosmeceutical composition disclosed herein is administered by a treating clinician. In another example, the composition is self-administered by the subject.

In an embodiment, treatment involves more than one dose (or application), of the composition disclosed herein, the pharmaceutical composition disclosed herein, or the cosmeceutical composition disclosed herein. For example, the compositions disclosed herein may be administered on a periodic basis such as, for example, daily, weekly, every other week or monthly. In some embodiments, the compositions disclosed herein are administered at least once daily for a period of: at least 1 month, at least 2 months, at least 3 months, at least 4 months, at least 5 months, at least 6 months, at least 7 months, at least 8 months, at least 9 months, at least 10 months, at least 11 months, or at least 12 months. In some embodiments, the compositions disclosed herein are administered at least once daily for a period of at least 18 months, at least 24 months, or at least 36 months.

In the contexts of the disclosed methods, ‘administering’ the compositions means applying the compositions disclosed herein for topical delivery of at least one bioactive compound to a required body surface area. In some embodiments, ‘applying’ includes applying the composition to a required body surface such as the skin, then rubbing or massaging the applied composition on the surface, for example by a circular or back and forth motion of the hand or an applicator. In some embodiments, the application is achieved by using an applicator. In some embodiments, the applicator is a textured applicator. In some embodiments, the applicator is a microtextured applicator. A microtextured applicator can be of various sizes suitable for desired surface areas, and made of different suitable materials, such as biocompatible and non-allergenic material, and may be manufactured, for example, using 3D printing. In some embodiments, the applicator is an applicator described in patent application WO 2014/094067 A1, the entire contents of which are incorporated herein by reference. In some embodiments, the compositions disclosed herein are delivered into the skin by rubbing or massaging on the skin using a microtextured applicator, such as the applicator described herein. For example, the applicator may be used to massage the topically applied composition on the skin, using circular motions or linear motions back and forth for a suitable period of time.

In some embodiments, the microparticles and the at least one bioactive compound are applied to a body surface (e.g., skin), as a single formulation. The formulation may be formed in advance and stored, or formed just prior to the application. Advantageously, the microparticles can be admixed with an existing or known formulation of the at least one bioactive compound.

In some embodiments, the at least one bioactive compound is formulated to be applied to a body surface (e.g., the skin), separately from the microparticles, such as after the application of the microparticles. For example, the at least one bioactive compound may be in the form of an existing or known formulation of the at least one bioactive compound.

The appropriate dose of the at least one bioactive compound to be administered as part of the compositions disclosed herein will be determined by a professional skilled in the art of topical treatments and/or hormone therapy. Generally, the dose will depend on the concentration of the bioactive in the composition, the amount of composition placed on the surface such as the skin, the area covered by the composition for topical delivery of the bioactive compound, and the frequency of application of the topical treatment.

In other embodiments, disclosed herein is a method of delivering at least one bioactive compound through the stratum corneum of skin on a subject for cosmeceutical purposes, excluding skin malignancies, the method comprising applying a composition administering the composition as defined herein, the pharmaceutical composition as defined herein, or the cosmeceutical composition as defined herein, to the subject, wherein the bioactive compound is optionally estradiol, optionally in the presence of further hormonal modulators.

The microparticles of the compositions disclosed herein may be eliminated or dissolved after application. Depending on the composition of the microparticles, the microparticles that penetrate the skin barrier may remain therein for 1, 2 or 3 weeks and be removed by the natural skin turnover. For example, it has been previously shown that the microparticles made of silica are well tolerated in human patients, with the microparticles eventually sloughing off.

### EXAMPLES

### Example 1—Production of Foroderm Microparticles

In some embodiments, the microparticles of the compositions disclosed herein are termed Foroderm. Examples of production of Foroderm microparticles are provided below.

Twelve wafers of silicon in the form of micropillars were fabricated by the South Australian Node of the Australian National Fabrication Facility (ANFF-SA). Four of 12 μm diameter, four of 9 μm diameter, and four of 6 μm diameter micropillars with the target height of 115 μm±10%, were produced.

Microparticles referred to as Fiberglass Foroderm were manufactured from silicon wafer called ‘etched microparticles’. A photomask was designed and fabricated to cover a 5″ diameter of a 6″ silicon wafer. The wafer was coated with photoresist via spin coating. Bosch etching was used to generate pillars of 10 μm diameter and 150 μm height. Each etched wafer cleaned to remove the photoresist prior to harvesting the pillars from the wafer to generate microparticles.

The etched microparticles (Foroderm) were removed from the silicon wafer using a blade and the surface of the wafer was washed with absolute ethanol. This procedure was repeated twice to collect the majority of the microparticles. The microparticles were collected in a 15 mL tube and centrifuged at 2500 rpm for 5 minutes. The clear supernatant was removed, and the precipitant (microparticles) was washed three times using acetone, followed by three washes in absolute ethanol and the last three washes with distilled water. For each washing, 5 ml solvent was added, and gently shaken followed by centrifugation at 2500 rpm for 5 minutes. The clear supernatant was removed each time and replaced by the fresh washing solvent. After the final washing procedure, the wet microparticles were transferred to pre-weighed 1.5 mL microtubes and left in the incubator at 60° C. for 24 hours. Several weight measurements of microtubes were performed until no weight changes were observed. Dry microparticles were stored at room temperature for future work and their sterilisation.

To determine the size of microparticles, brightfield images were taken and the images were analysed by ImageJ.

A total of 291 mg of microparticles with diameter of 12 μm, 297 mg of microparticles with diameter of 9 μm, and 85 mg of microparticles with diameter of 6 μm were harvested from the corresponding wafers. Table 1 depicts the length of each Foroderm, the length of Foroderm in three diameter groups of size is almost similar to the target size provided by the ANFF-SA.

TABLE 1
Weight (mg) and length (μm) of Foroderm
harvested from wafers manufactured by ANFF-SA

| | Diameter- | Fabrication | | |
|---|---|---|---|---|
| | Wafer No- | Yield (%) by | | Length (μm) |
| Label | Depth | ANFF- SA | Weight (mg) | Mean ± SD |
| A 12-W2 | 12-W2-125 | 90 | 74 | 110 ± 12 |
| B 12-W3 | 12-W3-119 | 95 | 41 | 106 ± 14 |
| C 12-W4 | 12-W4-121 | 98 | 40 | 105 ± 13 |
| D 12-W5 | 12-W5-119 | 98 | 136 | 110 ± 12 |
| E 9-W1 | 9-W1-119 | 75 | 68 | 111 ± 13 |
| F 9-W2 | 9-W2-120 | 40 | 25 | 107 ± 11 |
| G 9-W3 | 9-W3-110 | 95 | 112 | 113 ± 13 |
| H 9-W4 | 9-W4-115 | 90 | 92 | 111 ± 13 |
| I 6-W1 | 6-W1-111 | 30 | 10 | 97 ± 11 |
| J 6-W2 | 6-W2-110 | 40 | 16 | 103 ± 11 |
| K 6-W | 6-W3-110 | 90 | 47 | 111 ± 13 |
| L 6-W4 | 6-W4-110 | 50 | 12 | 107 ± 11 |

### Example 2—Estradiol Assay Validation

Estradiol, 17 β-[4-¹⁴C](E2-14C), 0.01 mCi/mL and with a specific activity of 55 mCi/mmol was purchased from American Radiolabeled Chemicals Inc., USA.

Certain volumes of E2-14C stock solution including 1 and 2 μl (equivalent to 0.01 μCi and 0.02 μCi, respectively) were added to 1 mL of water, separately. Various volumes of the prepared E2-14C aqueous solution including: 10, 20, 30, 40, and 50 μL, were placed in 1.5 mL Eppendorf tubes with the addition of 1.2 mL scintillation fluid (Ultima Gold™ XR, Perkin Elmer Inc. USA) and vortexed thoroughly. Samples were then counted using a microplate counter, MicroBeta® (Model 2450, PerkinElmer, Singapore Pte. Ltd) for 4 minutes. The values of counts per minute (CPM) were converted to disintegrations per minute (DPM), followed by converting to μCi. Equation 1 was used to calculate the percentage of E2-14C efficiency (Equation 1).

$$Efficiency ( \% ) = \frac{founded μ Ci E 2 - 14 C}{initial added μ Ci E 2 - 14 C} \times 100 \qquad ( Equation 1 )$$

The radiolabelling efficiency of E2-14C was also determined in digested skin. A volume of 2 μl of the E2-14C stock solution was added to 1 mL of water. Two series of digested skin experiments were considered. The skin was biopsied using a microsampling device with a diameter of 6 mm. In series one, one human full-thickness skin biopsy was placed in a 4 mL scintillation tube followed by adding a 500 μL Solvable™ solution. Various volumes including: 10, 20, 30, 40, and 50 μL, of the prepared E2-14C aqueous solution were added to each tube and the samples were left in the incubator at 60° C. to digest skin overnight. In series two, two pieces of skin biopsies and 800 μL of Solvable™ were placed in each vial, and various volumes of prepared E2-14C aqueous solution were added to each tube and left in the incubator at 60° C. overnight.

To measure E2-14C amount in each tube, scintillation fluid was added to fill the 4 mL tube and vortexed vigorously. Samples were analysed using a microplate counter. The percentage efficiency was calculated using equation 1.

The radiolabelling efficiency in the E2-14C aqueous solution was shown as 40%. The radiolabelling efficiency in the presence of one piece of skin and two pieces of skin were 25% and 14%, respectively. These values were used in later experiments as counting efficiency to convert CPM to DPM (Equation 2).

$$D P M = \frac{C P M}{Counting Efficiency} \qquad ( Equation 2 )$$

### Example 3—Estradiol Assay Validation

Estradiol, 17 β-[4-¹⁴C](E2-14C), 0.01 mCi/mL and with an activity of 55 mCi/mmol was purchased from American Radiolabeled Chemicals Inc., USA. β-Estradiol (E2) was purchased from Sigma-Aldrich (Sigma-Aldrich Pty Ltd., NSW, Australia).

E2 is a poorly water-soluble drug with a molecular weight of 272.4 g mol⁻¹, and Log P 4 and a water solubility of 3.9 mg/L at 27° C. It is very soluble in ethanol, acetone, dioxane (https://pubchem.ncbi.nlm.nih.gov/compound/Estradiol #section).

To develop a validated assay of E2, the radiolabelled E2 was selected, and a calibration range of E2 spiked with digested skin was proposed to cover 1-1000 mol/cm² of skin (0-272.4 ng/cm²).

A series of E2 standard solutions (STD) were prepared in water:ethanol mixture in the range of 0-30 μg/mL. The proposed STD concentrations were based on a dose-volume application of 10 μL of the formulation per cm² of skin. Table 2 shows the stock solutions needed to prepare various STD solutions.

TABLE 2
Stock solutions of E2 in ethanol and E2-14C in water

| Stock | Concentration | | |
|---|---|---|---|
| solutions | (μg/mL) | Solvent | Preparation |
| Solution A | 1000 | Ethanol | Dissolve 5 mg E2 in ethanol |
| Solution B | 500 | Ethanol | 500 μL from solution A + |
| | | | 500 μL ethanol |
| Solution C | 50 | Ethanol | 100 μL from solution B + |
| | | | 900 μL ethanol |
| E2-14 C | 5.8 | Water | 70 μL from stock E2-14C + |
| solution | | | 600 μL water |

All stock solutions were mixed thoroughly using a vortex before using.

Table 3 shows the concentrations and the volume of each stock solution and water in the final STD solutions containing E2 and E2-14C.

TABLE 3
Volume of each stock solution, ethanol percentage, amount of E2-14C,
and the ratio of E2 and E2-14C in 1 mL of each calibration standard

| STD conc. | | Ethanol | E2-14C | Water | Ethanol | E2-14C | E2/ |
|---|---|---|---|---|---|---|---|
| (μg/mL) | | (μL) | (μL) | (μL) | (%) | (μg) | E2-14C |
| | Solution B | | | | | | |
| | (μL) | | | | | | |
| 30 | 60 | 40 | 60 | 840 | 10 | 0.350 | 86 |
| 27 | 54 | 46 | 54 | 846 | 10 | 0.315 | 86 |
| 25 | 50 | 50 | 50 | 850 | 10 | 0.292 | 86 |
| 20 | 40 | 60 | 40 | 860 | 10 | 0.233 | 86 |
| 15 | 30 | 70 | 30 | 870 | 10 | 0.175 | 86 |
| 10 | 20 | 80 | 20 | 880 | 10 | 0.117 | 86 |
| | Solution C | | | | | | |
| | (μL) | | | | | | |
| 5 | 100 | 0 | 100 | 800 | 10 | 0.583 | 8.6 |
| 3.5 | 70 | 30 | 70 | 830 | 10 | 0.408 | 8.6 |
| 2.7* | 54 | 46 | 54 | 846 | 10 | 0.315 | 8.6 |
| 2.5 | 50 | 50 | 50 | 850 | 10 | 0.292 | 8.6 |
| 1 | 20 | 80 | 20 | 880 | 10 | 0.117 | 8.6 |
| 0.5 | 10 | 90 | 10 | 890 | 10 | 0.058 | 8.6 |
| 0 | 0 | 100 | 0 | 900 | 10 | 0 | 0.0 |

*10 μL of this solution is equivalent to 27 ng.

A volume of 100 μL of each STD solution was transferred into a 1.5 mL Eppendorf tube followed by adding 1.2 mL scintillation fluid (Ultima Gold™ XR, Perkin Elmer Inc. USA) and vortexed thoroughly. Samples were then counted using a microplate counter, MicroBeta® (Model 2450, PerkinElmer, Singapore Pte. Ltd) for 4 min.

The radiolabelling efficiency of the E2-14C stock solution in water was also calculated (Equation 1) to be used to convert CPM to DPM (Equation 2). The DPM values were used to calculate the E2-14C activity based on Ci followed by the amount (μg) of E2-14C (Equations 3 and 4). Equation 5 was used to calculate the final E2 in each STD.

$$Efficiency ( \% ) = \frac{founded μ Ci E 2 - 14 C}{initial added μ Ci E 2 - 14 C} \times 100 \qquad ( Equation 1 )$$

$$D P M = \frac{C P M}{Counting Efficiency} \qquad ( Equation 2 )$$

$$μ Ci = ( D P M \times 4.5 \times {10}^{- 13} ) \times {10}^{- 6} \qquad ( Equation 3 )$$

$$Amount ( μ g E 2 - 14 C ) = ( \frac{μ Ci}{Specific Activity} ) \times Molecular weight \qquad ( Equation 4 )$$

$$Total E 2 = E 2 - 14 C amount \times ( \frac{E 2}{E 2 + 14 C} ) + E 2 - 14 C amount \qquad ( Equation 5 )$$

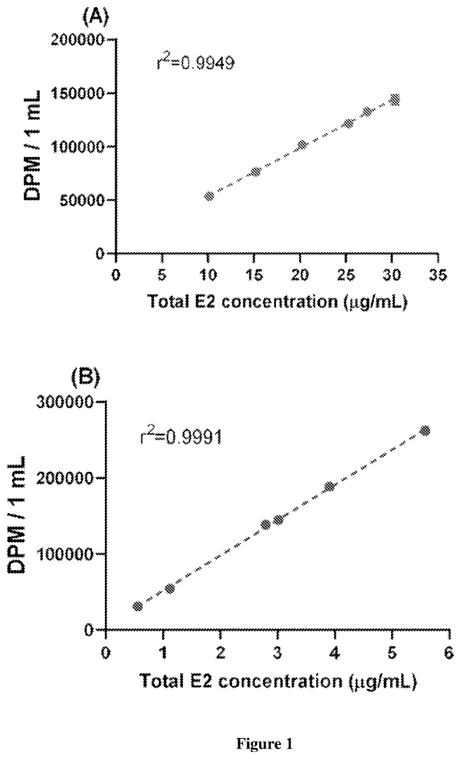

The counting efficiency was 23% and it was used to convert CPM to DPM. FIG. 1 shows the plots of two different ranges of E2 STD (A: 10-30 μg/mL) with the E2 to E2-14C ratio of 86 (Table 3) and FIG. 1 B (0.5-5 μg/mL) with the E2 to E2-14C ratio of 8.6 (Table 3). Both plots show a good correlation with the r² of about 0.99.

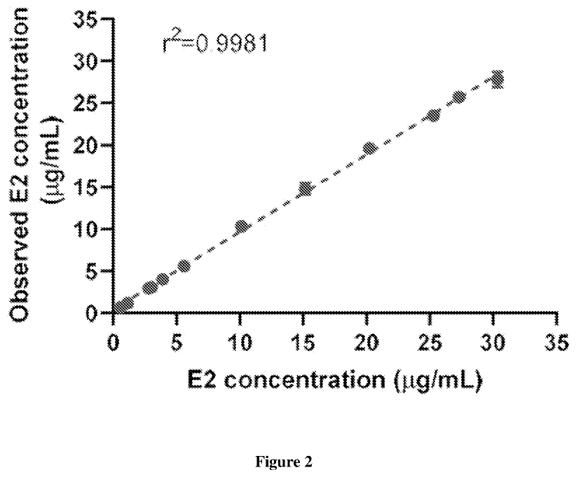

Total E2 concentration in each STD was calculated using equations 2 to 5 and was plotted versus E2 STD concentrations (Table 3) and is shown in FIG. 2. There is a good correlation between the two concentrations which suggests that radiolabelled E2 can be used for sensitive quantification of E2 in the water-ethanol mixture.

### Example 4—Estradiol Assay Validation in Digested Skin

To prepare a calibration range of E2 spiked with skin with the range of 0-272.4 ng/cm², 12 calibration standards of E2 were prepared in the range of 0-30 μg/mL in water:ethanol (90:10). In the next step, three pieces of skin biopsies were placed in a 4 ml scintillation tube followed by adding a 1.5 ml Solvable™ and left in an incubator at 60° C. overnight, 14 digested skins were prepared. Once the digested skins were ready, 10 μL of each STD in a water-ethanol mixture was added to 1.5 mL of digested skin (Table 4). The total volume of 1.5 mL E2 spiked with digested skin was divided into 700 μL volumes and placed in separate 4 mL scintillation tubes followed by adding scintillation liquid (Ultima Gold™ XR, Perkin Elmer Inc. USA). Two digested skin samples were controlled without adding E2. All tubes were vortexed vigorously and the content of E2-14C was measured by microplate counter, MicroBeta® (Model 2450, PerkinElmer, Singapore Pte. Ltd) for 4 minutes. The radiolabelling efficiency of the E2-14C in digested skin was also calculated (Equation 1) and used as counting efficiency to convert CPM to DPM (Equation 2). The DPM values were used to calculate the E2-14C activity based on μCi followed by the amount (μg) of E2-14C (Equations 3 and 4). Equation 5 was used to calculate the final E2 in each STD.

TABLE 4
Preparation of E2 spiked with digested skin

| STD | STD | | Digested | | |
|---|---|---|---|---|---|
| Conc. | (E2 + E2-14C) | E2 | Skin (mL) | E2-14C | E2 / |
| (ng/mL) | Solution (μL) | (ng/10 μL) | (3 pieces) | (ng) | E2-14C |
| 30000 | 10 | 300 | 1.5 | 3.5 | 85.7 |
| 27000 | 10 | 270 | 1.5 | 3.2 | 85.7 |
| 25000 | 10 | 250 | 1.5 | 2.9 | 85.7 |
| 20000 | 10 | 200 | 1.5 | 2.3 | 85.7 |
| 15000 | 10 | 150 | 1.5 | 1.8 | 85.7 |
| 10000 | 10 | 100 | 1.5 | 1.2 | 85.7 |
| 5000 | 10 | 50 | 1.5 | 5.8 | 8.6 |
| 2700 | 10 | 27 | 1.5 | 4.1 | 8.6 |
| 2500 | 10 | 25 | 1.5 | 3.2 | 8.6 |
| 1000 | 10 | 10 | 1.5 | 2.9 | 8.6 |
| 500 | 10 | 5 | 1.5 | 1.2 | 8.6 |
| 0 | 10 | 0 | 1.5 | 0 | 8.6 |

The target skin dose in this study expected to be delivered and measured is highlighted in Table 3. As an example of spiked solution preparation, from the STD solution of 2700 ng/mL (2.7 μg/mL), 10 μL containing 27 ng E2 and 4.1 ng E2-14C was spiked with the digested skin.

The range of E2-14C in spiked solutions is between 1.2 ng/1.5 mL to 5.8 ng/1.5 mL.

The counting efficiency of E2-14C in the presence of digested skin was 17% and it was used to calculate DPM (Equation 2), and the total E2 recovered in each E2 spiked with skin sample using the Equations 3, 4 and 5.

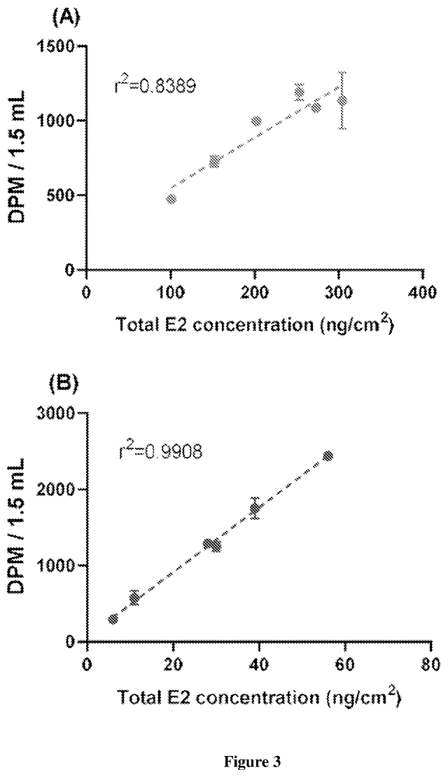

According to FIG. 3A, radiolabelled E2 showed a limited capacity to be used in high concentrations of E2 (r²=0.8389). This partial failure can be explained by the limited solubility of E2 in aqueous solutions and digested skin and hence, precipitating of E2 in the final solutions. However, at lower concentrations (FIG. 3B), a good linear relationship between E2 concentrations and DPM was observed (r²=0.99).

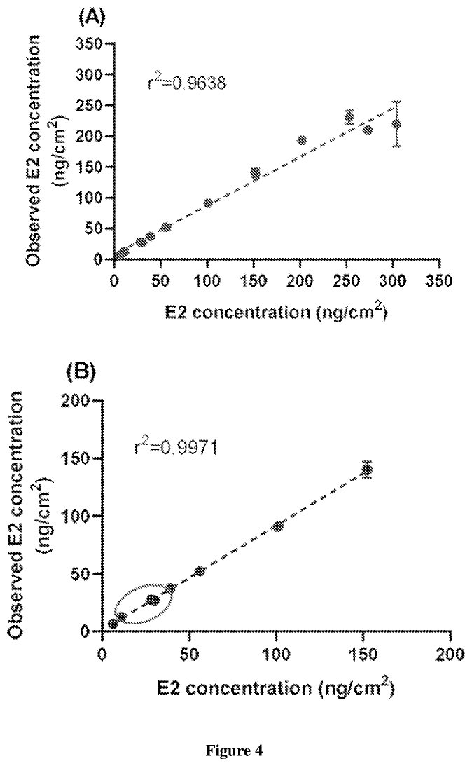

According to FIG. 4A, the four upper concentrations of E2 are not aligned with the rest of the E2 STD spiked with skin. However, a good correlation (r²=0.99) between E2 STD concentration and observed E2 concentration spiked with digested skin at a defined range of 5-150 ng/cm² could be observed (FIG. 4B). Considering the point that the skin target dose is defined as 27.2 ng/cm², radiolabelled E2 can be used as a sensitive and specific assay method in the present study.

### Example 5—Estradiol Assay Validation in Tape Stripped Skin

Estradiol, 17 β-[4-¹⁴C](E2 14C), 0.01 mCi/mL and with an activity of 55 mCi/mmol was purchased from American Radiolabeled Chemicals Inc., USA. β-Estradiol (E2) was purchased from Sigma-Aldrich (Sigma-Aldrich Pty Ltd., NSW, Australia).

A solution of E2 14C was prepared by adding 6 μL of E2 14C to 100 μL of water:ethanol (9:1) and mixed thoroughly. The prepared solution contains 300 ng E2 14C. Another solution was prepared by adding 3 μL of E2 14C to 100 μL of water:ethanol (9:1) containing 1.5 μg/mL E2. The prepared solution contained 150 ng E2 14C and 150 ng E2.

Full-thickness human skin was obtained from a female patient (40 years old) after surgical abdominoplasty. The skin was marked with a surface area of 1.3 cm² followed by 20 times of tape stripping (D-Squame tape, 22 mm diameter) to remove the stratum corneum. The prepared E2 solutions were applied on tape stripped skin and left in the water bath for 90 minutes at 37° C. At the end of the experiment, residual materials were removed from the surface by wetting cotton swabs three times. The skin was then biopsied using a microsampling device with a diameter of 6 mm. Each piece of biopsy was placed in a 4 mL scintillation tube followed by adding 500 μL of the Solvable™ solution (Perkin Elmer Inc. USA). The samples were left in the incubator at 60° C. to digest skin overnight. To measure the E2 14C amount in each, scintillation fluid (Ultima Gold™ XR, Perkin Elmer Inc. USA) was added to fill the 4 mL tube and vortexed vigorously. Samples were analysed using a microplate counter, MicroBeta® (Model 2450, PerkinElmer, Singapore Pte. Ltd). The experiment was done in triplicates.

Table 5 shows the CPM obtained from analysing one skin biopsy with 0.28 cm². To calculate DPM, a counting efficiency of 25% was considered.

TABLE 5
Results obtained after applying 10 μL/cm² of E2 (in water-ethanol) on
tape stripped skin

| | | | μCi/ | ng/Biopsy | Total E2 |
|---|---|---|---|---|---|
| | CPM | DPM | Biopsy | (E2-14C) | (ng/Biopsy)* |
| E2-14C | 1365 | 5337 | 0.002402 | 11.9 | 11.9 |
| | 940 | 3760 | 0.001692 | 8.4 | 8.4 |
| | 869 | 3476 | 0.001564 | 7.7 | 7.7 |
| E2-14C + | 416 | 1664 | 0.000749 | 3.7 | 7.4 |
| E2 | 361 | 1444 | 0.00065 | 3.2 | 6.4 |
| | 468 | 1872 | 0.000842 | 4.2 | 8.3 |

$* Total E 2 = E 2 - 14 C amount \times ( \frac{E 2}{E 2 - 14 C} ) + E 2 - 14 C amount \qquad ( Equation 6 )$

A target of CPM of 100 is considered for the experiment. CPM values are high since the stratum corneum as the main barrier was removed in this experiment. In the presence of stratum corneum a significant reduction in CPM values was expected, therefore, incorporating at least 6 μL of stock E2-14C in 100 μL of the formulation seems reasonable. Reducing the volume of E2-14C in the solution containing E2-14C+E2 resulted in almost half of CPM measured from the E2-14C solution indicating that radiolabelled E2-14C is sensitive and specific to quantifying E2 delivery.

### Example 6—Foroderm Coating Characterisation

β-Estradiol (E2), alginic acid sodium salt from brown algae, low viscosity (Na Alg), calcium chloride (CaCl₂)), and carboxymethyl cellulose sodium (low viscosity) were purchased from Sigma-Aldrich (Sigma-Aldrich Pty Ltd., NSW, Australia).

Na alginate (Na Alg), one of the most commonly used natural polysaccharides, was used as a coating excipient for the Foroderm. Na alginate solution (1%) was prepared by adding 100 mg of Na in the water:ethanol (90:10) mixture containing 25 μg/mL E2, with continuous stirring using a magnetic stirrer bar for about 30 minutes at 400 rpm to completely dissolve the alginate.

A solution of Na Alg (1%) containing 12.5 μg/mL of E2 was also prepared by dissolving 100 mg of Na Alg in 10 mL water:ethanol and was used for the comparison.

Gelation of Na Alg can be achieved by introducing cations such as Ca²⁺ to form gels by substitution of the sodium ions (Abbasalizade et al., 2020). CaCl₂) solution was made by dissolving appropriate amounts of CaCl₂) in water to obtain the desired concentration of 0.2, 0.4, 0.6, 0.8, 1, and 2 mg/mL. The cross-linked gel was obtained by adding CaCl₂) solutions to the Na Alg (1%) solution with the volume ratio of 1:1 and vortexed at 2000 rpm for 10 seconds. The final concentration of Na Alg in the prepared gel was 0.5% while the concentrations of CaCl₂) were in the range of 0.1-1 mg/mL. All prepared gels contained 12.5 μg/mL of E2. Na Alg solution with 0.5% was made by adding 1 mL water:ethanol mixture.

Various carboxymethylcellulose (CMC) gels (0.5, 1, 1.5, 2, 2.5 and 5%) were prepared by adding the certain amount of CMC in water:ethanol (90:10) mixture containing 12.5 μg/mL of E2. The mixture was mixed continuously using a magnetic stirrer bar at 300 rpm and room temperature until a transparent gel was obtained.

Fiberglass Foroderm with a length of 124.3±56.0 μm was used to be coated by three prepared coating materials. An amount of 10 mg Foroderm was added to 50 L of Na Alg solution, Na Alg cross-linked gel, and CMC gels to prepare the freeze-dried samples. Samples were then mixed with Foroderm using a vortex and frozen immediately in the liquid nitrogen for a few seconds. The frozen samples were transferred to the freeze-drying machine and left overnight. The solution of water:ethanol (90:10) with 12.5 μg/mL E2 was used as a control. All samples for freeze-drying were prepared in duplicates. The ease of freeze-dyed samples to be disintegrated was assessed by fine touching of samples by fingers to break them down and imaged under light microscopy.

The coated particles were subjected to study by light microscopy and brightfield microscopy. Scanning electron microscopy (SEM) (Zeiss Merlin FEG SEM with SDD EDS) was performed to characterise the morphology and particle coverage of Foroderm. Freeze-dried samples were placed on a carbo-coated copper grid. An ultra-thin coating (1 nm) of electrically conducting metal was applied. The SEM images were obtained with EHT of 2 KV, and two different magnifications of 100× and 1500×.

All CMC gels were homogenous and runny, however, the viscosity increased as the CMC concentrations increased.

The first consideration was to evaluate the ease of the disintegration of freeze-dried samples by small pressure of fingers. The ease of disintegration was as follow: E2 solution>Na Alg (0.5%)>Na Alg (0.5%)+CaCl₂) (0.1 mg·mL)>Na Alg (0.5%)+CaCl₂) (0.2 mg·mL)>Na Alg (0.5%)+CaCl₂) (0.3 mg·mL)>Na Alg (0.5%)+CaCl₂) (0.4 mg·mL)>Na Alg (0.1%)>Na Alg (0.5%)+CaCl₂ (0.5 mg·mL)>Na Alg (0.5%)+CaCl₂) (1 mg·mL).

The ease of disintegration test of CMC gels showed that only the gels with 0.5 and 1% could disintegrate with finger pressure and the rest of the samples remained as a clump of dry gel.

Considering the disintegration test and SEM images, to prepare dry formulations, Na Alg 0.5% solution, Na Alg 0.5% plus CaCl₂) 0.2 mg/mL and CMC 0.5% can be potential coating agents of the Foroderm.

### Example 7—Foroderm Delivery Characterisation

β-Estradiol (E2) was purchased from Sigma-Aldrich (Sigma-Aldrich Pty Ltd., NSW, Australia), Estradiol, 17 β-[4-¹⁴C](E2-14C), 0.01 mCi/mL and with a specific activity of 55 mCi/mmol was purchased from American Radiolabeled Chemicals Inc., USA.

An ethanol-water mixture (30:70) was selected to dissolve E2. Three various E2 concentrations including 5, 10, and 30 μg/mL were prepared. A volume of 24 μL of E2-14C stock solution was added to 400 μL of the ethanol-water mixture (30:70) containing E2 and mixed thoroughly.

An abdominal full-thickness human skin from female 26 years old was used. The skin was stained with an aqueous methylene blue solution (50 μg/mL) by immersing the skin for 5 minutes followed by washing under the stream of water for 1 minute and dried gently using tissue. The skin was pinned on a foam board with a moist cloth underneath. The undamaged area of skin was marked with a surface area of 2×2 cm and left in the water bath set at 37° C. for 30 minutes. A volume of 50 μL of ethanolic solutions of E2 (10 and 30 μg/mL) and E2-14C with 5 mg Fiberglass Foroderm (Applicator+Foroderm; ‘Foroderm’) and without Foroderm (Applicator only; ‘control’) was massaged on the skin in a small circular motion for 30 seconds by a microtextured pen applicator. After dosing the skin with formulations, the foam was floated at the surface of the covered water bath at 37° C. (skin surface 32° C.) for 90 minutes. At the end of the experiment, residual materials on the skin surface were removed by wetting cotton swabs followed by 5 times tape stripping (D-Squame tape, 22 mm diameter). The skin was then biopsied using a microsampling device with a diameter of 6 mm. One biopsy from the group with Foroderm and two pieces of biopsy from the group without Foroderm were placed in a 4 mL scintillation tube followed by adding 500 μL and 800 μL of the Solvable™ solution (Perkin Elmer Inc. USA) to the samples respectively. The samples were left in the incubator at 60° C. to digest skin overnight. To measure the E2-14C amount in each, scintillation fluid (Ultima Gold™ XR, Perkin Elmer Inc. USA) was added to fill the 4 mL tube and vortexed vigorously. Samples were analysed using a microplate counter, MicroBeta® (Model 2450, PerkinElmer, Singapore Pte. Ltd). The experiment was done in triplicates. The experiment was performed to test the initial dose E2 of 5 μg/mL but the volume application dose was reduced to 40 μL on 4 cm². All skin experiments were done in triplicates.

Table 6 shows the E2 dose applied to the skin surface area and the final percentage dose of total E2 in the solution.

TABLE 6
Prepared E2 solutions and their dose application to the skin

| E2 Conc. (μg/mL) | Amount | Amount E2- | | Total E2 in |
|---|---|---|---|---|
| Prepared in | E2/cm² | 14C/cm² | | Solution |
| Eth:Water (30:70) | (ng) | (ng) | E2/E2-14C | (%) |
| 5 | 50 | 29.7 | 1.7 | 0.0008 |
| 10 | 125 | 37.1 | 3.4 | 0.0013 |
| 30 | 375 | 37.1 | 10.1 | 0.0033 |

The amount that penetrated the skin increased as the initial concentration of E2 increased. After applying of solution with 10 μg/ml of E2, about 50 ng of E2 in total was found in the skin. Therefore, the third concentration of E2 (5 μg/mL) was also tested and resulted in a mean value of 37 ng/cm² (Table 7). The target dose was proposed as 27.2 ng/cm², however, it was decided to proceed with this E2 concentration to study the effect of chemical penetration enhancers combined with Foroderm.

TABLE 7
Total E2 amount penetrated skin (ng/cm²) after treating skin
with/without Foroderm. Experiments were done in triplicates.

| | With Foroderm | | | | Without Foroderm | | | |
|---|---|---|---|---|---|---|---|---|
| E2 conc. | 1 | 2 | 3 | Mean ± SD | 1 | 2 | 3 | Mean ± SD |
| 5 μg/mL | 37.8 | 32.1 | 43.5 | 37.8 ± 5.7 | 5.6 | 7.8 | 7.6 | 7.0 ± 1.2 |
| 10 μg/mL | 49.4 | 51.5 | 55.0 | 52.0 ± 2.8 | 9.6 | 15.8 | 9.6 | 11.7 ± 3.6 |
| 30 μg/mL | 133.9 | 97.5 | 93.9 | 108.4 ± 22.1 | 27.2 | 28.8 | 20.1 | 25.4 ± 4.6 |

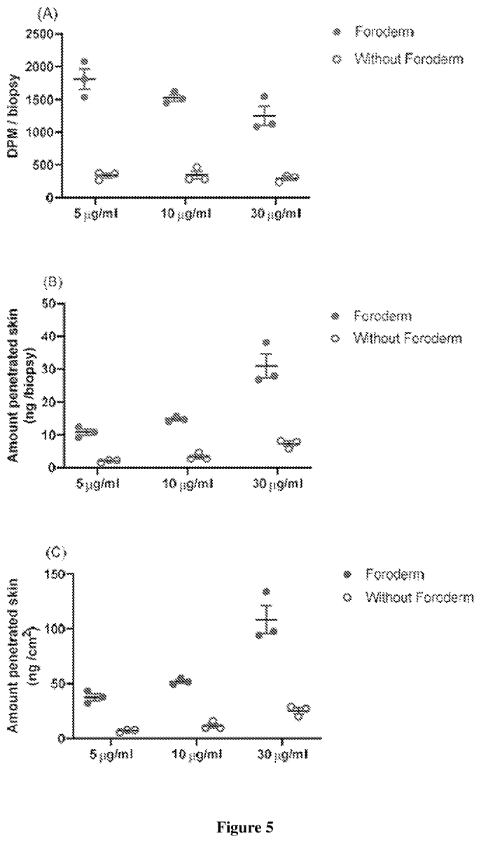

FIG. 5 shows the DPM found in each biopsy and the calculated amount considering the ratio of E2/E2-14C in the solutions.

### Example 8—Preparation of Liquid Topical Formulations Containing Chemical Penetration Enhancers

A series of chemical penetration enhancers (CPE) were selected to prepare topical formulations to be tested as chemical penetration enhancers on the skin and in combination with Foroderm as a physical penetration enhancer. Formulations were prepared with and without E2.

β-Estradiol (E2), dimethyl sulfoxide, ethanol, propylene glycol, isopropyl alcohol, urea, sodium dodecyl sulfate R-(+)-limonene, isopropyl myristate, oleic acid, undecanoic acid were purchased from Sigma-Aldrich (Sigma-Aldrich Pty Ltd., NSW, Australia). Tween® 80 was purchased from Chem-supply (Chem-supply Pty Ltd, SA, Australia) and menthol was provided by Plant essentials (QLD, Australia).

Estradiol, 17 β-[4-¹⁴C](E2-14C), 0.01 mCi/mL and with a specific activity of 55 mCi/mmol was purchased from American Radiolabeled Chemicals Inc., USA.

Table 8 shows the CPE with the percentages used. The percentages of each CPE in the formulation were selected in such a way that they are below the maximum potency per unit dose provided by the Food and Drug Administration (FDA), (https://www.accessdata.fda.gov/scripts/cder/iig/index.cfm).

TABLE 8
Percentage of chemical penetration enhancers
used to prepare liquid topical formulations

| | | CPE in | |
|---|---|---|---|
| | | the Final | Maximum potency |
| | | Formulation | per unit |
| Name | Abbreviation | (%) | dose (%)^{a} |
| Ethanol | Eth | 30 | 25-92.38 |
| Dimethyl sulfoxide | DMSO | 5 | 45.5 |
| Propylene glycol | PG | 30 | 1.6-30 |
| Isopropanol | IPA | 30 | 20-40 |
| Tween 80 | T80 | 5 | 2.5-15 |
| Urea | Urea | 5 | 4.2 |
| Sodium dodecyl | SDS | 0.5 | 0.05-1 |
| sulfate | | | |
| Menthol | Ment | 0.1 | 0.08-0.1 |
| Limonene | Lim | 5 | 0.5-10 |
| Oleic acid | OA | 5 | 7.4-25 |
| Isopropyl Myristate | IPM | 5 | 5-41.3 |
| Undecanoic acid | UDA | 5 | — |

^{a}It is defined based on the dosage forms of topical products.

To prepare 1 mL of each CPE solution, CPE was first dissolved in the proper solvent (Table 9) and the rest of the ingredients were added afterward followed by mixing the solution for 30 seconds using a vortex.

In the case of urea and SDS, specific weights were dissolved in water before adding ethanol. Due to the small percentage of menthol in the final formulation, a solution of 1% menthol in ethanol was prepared and used to be added to the final formulation.

Formulations containing E2 were prepared by preparing an ethanolic solution of 100 μg/mL E2. The volume/amount of each ingredient is presented in Table 8. The final E2 concentration in each CPE solution is 5 μg/mL and 30% ethanol.

Table 9 shows the final composition of each formulation in 1 mL of the formulation with and without E2 are presented.

TABLE 9
Composition of 1 mL of formulation containing CPE and E2

| | | CPE in Final | | | | |
|---|---|---|---|---|---|---|
| | | Formulation | CPE | E2-Eth | | |
| | | v/v or w/v | Needed | (100 μg/mL) | Ethanol | water |
| CPE | Solvent | (%) | μl or mg | μL | μL | μL |
| Eth | Water | 30 | 300 | 50 | 250 | 700 |
| DMSO | | 5 | 50 | 50 | 250 | 650 |
| PG | | 30 | 300 | 50 | 250 | 400 |
| IPA | | 30 | 300 | 50 | 250 | 400 |
| T80 | | 5 | 50 | 50 | 250 | 650 |
| Urea | | 5 | 50 | 50 | 250 | 700 |
| SDS | | 0.5 | 5 | 50 | 250 | 700 |
| Ment | | 0.1 | 100 (1%) | 50 | 150 | 700 |
| Lim | Ethanol | 5 | 50 | 50 | 250 | 650 |
| OA | | 5 | 50 | 50 | 250 | 650 |
| IPM | | 5 | 50 | 50 | 250 | 650 |
| UDA | | 5 | 50 | 50 | 250 | 650 |

### Example 9—Evaluation of Various Chemical Penetration Enhancers on Foroderm Enhanced Estradiol Delivery

An abdominal full-thickness human skin from female 26 years old was used. An ethanolic-water solution was selected as a control.

β-Estradiol (E2), Dimethyl sulfoxide, ethanol, propylene glycol, isopropyl alcohol, urea, sodium dodecyl sulfoxide, R-(+)-limonene, isopropyl myristate, oleic acid, undecanoic acid were purchased from Sigma-Aldrich (Sigma-Aldrich Pty Ltd., NSW, Australia). Tween® 80 was purchased from Chem-supply (Chem-supply Pty Ltd, SA, Australia) and menthol was provided by Plant essentials (QLD, Australia).

Estradiol, 17 β-[4-¹⁴C](E2-14C), 0.01 mCi/mL and with a specific activity of 55 mCi/mmol was purchased from American Radiolabeled Chemicals Inc., USA.

A volume of 18 μL of E2-14C stock solution was added to 300 μL of liquid formulations containing CPE (Table 10) prior to the skin evaluation tests and mixed thoroughly.

The skin was stained with an aqueous methylene blue solution (50 μg/mL) by immersing the skin for 5 minutes, followed by washing under the stream of water for 1 minute and dried gently using tissue. The skin was pinned on a foam board with a moist cloth underneath. The undamaged area of skin was marked with a surface area of 2×2 cm and left in the water bath set at 37° C. for 30 minutes. The transepidermal water loss (TEWL) of each marked area was measured three times using a vapometer (Delfin Technologies Ltd, Finland). A volume of 40 μL of various CPE solutions containing E2-14C and E2 with 5 mg Foroderm (Applicator+Foroderm) and without Foroderm (Applicator only) was massaged on the skin in a small circular motion for 30 seconds by a microtextured pen applicator. After dosing the skin with formulations, the foam was floated at the surface of the covered water bath and sat at 37° C. (skin surface 32° C.) for 90 minutes. At the end of the experiment, residual materials on the skin surface were removed by wetting cotton swabs. The TEWL was measured followed by 5 times tape stripping (D-Squame tape, 22 mm diameter). The skin was then biopsied using a microsampling device with a diameter of 6 mm. One biopsy from the group with Foroderm and two pieces of biopsy from the group without Foroderm were placed in a 4 mL scintillation tube followed by adding 500 μL and 800 μL of the Solvable™ solution (Perkin Elmer Inc. USA) to the samples respectively. The samples were left at the incubator at 60° C. to digest skin overnight. To measure the E2-14C amount in each, scintillation fluid (Ultima Gold™ XR, Perkin Elmer Inc. USA) was added to fill the 4 mL tube and vortexed vigorously. Samples were analysed using a microplate counter, MicroBeta® (Model 2450, PerkinElmer, Singapore Pte. Ltd). The experiment was done in triplicates.

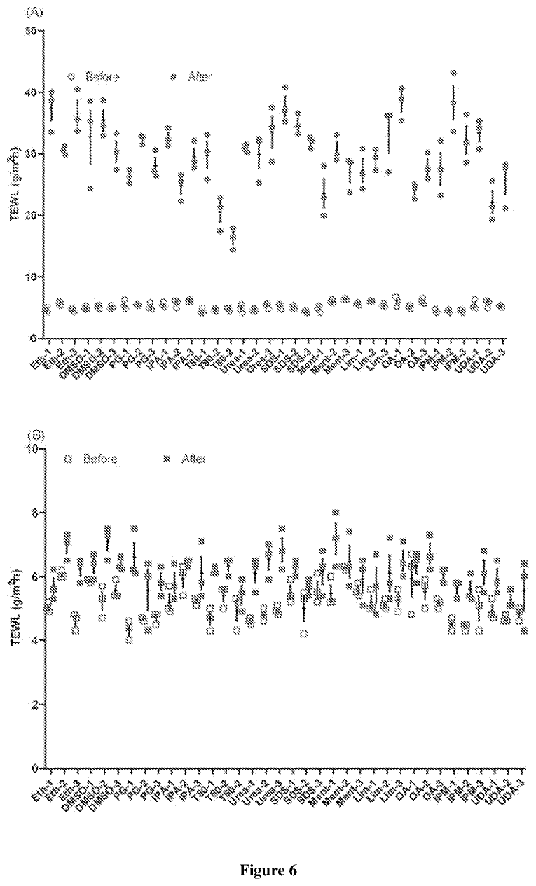

The TEWL of human skin measured before and after treatment is shown in FIG. 6. Treatment of skin with Foroderm resulted in an increase in TEWL (FIG. 6A) while there were no significant changes after treatment skin with CPE formulation and applicator only (FIG. 6B).

The total E2 amount that penetrated skin (ng/cm²) is presented in Table 10. Because of high variability in the amounts obtained from PG skin samples, this penetration enhancer was repeated.

Results showed that Foroderm increased the penetration of E2 compared to the corresponding control group. Among the CPE plus Foroderm, ethanol 30% (alone), DMSO and PG showed the highest amount penetrated skin with Foroderm. Within the group of CPE without Foroderm, ethanol, limonene showed the highest E2 amount in skin.

TABLE 10
Total E2 amount penetrated skin (ng/cm²) after treating skin
with/without Foroderm. Experiments were done in triplicates.

| Chemical | | | | | | | | |
|---|---|---|---|---|---|---|---|---|
| Penetration | With Foroderm | | | | Without Foroderm | | | |
| Enhancers | 1 | 2 | 3 | Mean ± SD | 1 | 2 | 3 | Mean ± SD |
| Eth | 37.8 | 32.1 | 43.5 | 37.8 ± 5.7 | 5.6 | 7.8 | 7.6 | 7.0 ± 1.2 |
| DMSO | 34.8 | 48.9 | 37.5 | 40.4 ± 7.5 | 7.9 | 5.4 | 5.8 | 6.4 ± 1.3 |
| PG | 63.5 | 33.8 | 49.0 | 50.0 ± 10.2 | 3.5 | 2.2 | 2.9 | 2.9 ± 0.7 |
| | 54.5 | 55.0 | 44.3 | | | | | |
| IPA | 19.8 | 27.9 | 24.3 | 24.0 ± 4.1 | 3.4 | 4.2 | 4.1 | 3.9 ± 0.4 |
| T80 | 4.9 | 4.0 | 4.6 | 4.5 ± 0.5 | 1.2 | 1.3 | 1.0 | 1.2 ± 0.2 |
| Urea | 33.8 | 22.2 | 19.3 | 25.1 ± 7.7 | 6.8 | 8.4 | 4.9 | 6.7 ± 1.8 |
| SDS | 13.9 | 14.3 | 19.4 | 15.9 ± 3.1 | 2.5 | 4.0 | 5.3 | 4.0 ± 1.4 |
| Ment | 24.2 | 14.3 | 18.7 | 19.1 ± 5.0 | 5.7 | 8.5 | 7.5 | 7.2 ± 1.4 |
| Lim | 17.1 | 22.3 | 19.3 | 19.6 ± 2.6 | 12.2 | 9.7 | 11.2 | 11.0 ± 1.3 |
| OA | 21.1 | 11.5 | 16.2 | 16.3 ± 4.8 | 3.0 | 3.9 | 4.0 | 3.6 ± 0.6 |
| IPM | 19.2 | 17.5 | 13.5 | 16.7 ± 2.9 | 4.3 | 4.7 | 4.7 | 4.5 ± 0.2 |
| UDA | 11.8 | 8.8 | 12.7 | 11.1 ± 2.1 | 2.0 | 3.2 | 2.3 | 2.5 ± 0.6 |

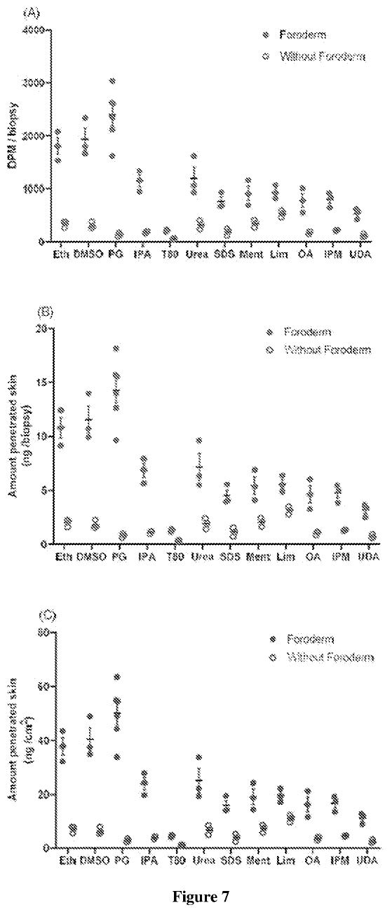

FIG. 7 shows the DPM found in each biopsy and the calculated amounts of E2 considering the ratio of E2/E2-14C in the CPE solutions in two groups.

### Example 10—Clinical Trial Investigating Topical Administration of Estradiol-Containing Foroderm Compositions to Postmenopausal Women

### Methods

The trial was undertaken in July 2022 at a specialist women's health clinic associated with the University of Adelaide, South Australia. Three postmenopausal women attending the clinic were chosen as per inclusion/exclusion criteria including not receiving any dermal or systemic hormonal therapies and a serum estradiol levels less than 88 pmol/L, the minimal level of detection of the radio immunoassay. Skin thickness and stiffness evaluation was performed using the Aixplorer ultrasound system (Supersonic Imagine, France) equipped with a 4-15-MHz linear probe. Utilising this scan technique the mean skin thickness for a 54 year old woman is (0.9-3.3 mm) and skin elasticity 13-32 kPa. Dermoscopic photography was undertaken at baseline and at each of the subsequent blood draws at 4, 24, 72 and 168 hours. 5 mg of microparticles (diameter of 9 μm and a length of 113±13 μm) was applied to the skin followed by applying 70 μl of formulation (30% ethanol, 5% isopropyl myristate, 0.1% menthol, 0.001% of Estradiol) to the same area. Microtextured curved applicator heads were printed using a 3D-printer in order to create convex surface. ‘Microtextured’ means that the surface has raised and/or lowered surface features on the micrometre to millimetre scale. Then, immediately an applicator was used to massage the skin for 30 seconds. The excess of microparticles and formulation were removed by alcohol wipes. A final blood draw was undertaken at 40 days to ensure that there was no delayed Depo release from the microparticle insertion. A single dose of topical application of microparticles coated with estradiol (0.4 g) applied once to a 4 cm² area from the inferior aspect of the earlobe to the angle of the jaw. The microparticles (silica) were fabricated by deep reactive ion etching in ISO certified clean room. The length is the measured using microscopy. The dose of estradiol was chosen to replicate the preclinical evaluation of skin concentration achieved by the application of 50 mcg (micrograms) of estradiol, the dose commonly used in application for hormone replacement therapy (HRT).

### Results

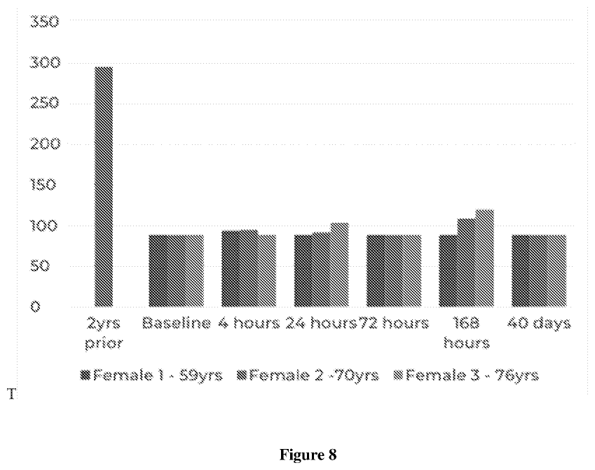

The mean age of the female patients was 68.5 years old and the mean years post-menopause was 16.5 years. Shear wave ultrasound revealed the mean skin thickness was 0.65 mm and the skin elasticity was 11.2 kPa, which is extremely thin and fragile skin. The measured serum estradiol levels are shown in Table 11 and FIG. 8. The results show there was no elevation of estradiol beyond what is expected in a postmenopausal woman. In comparison, patient #2, who 2 years prior to the study, had received Estradot® (50 mcg estradiol; 2× per week) for transdermal HRT (the dose replicating that used in the trial) demonstrated a serum estradiol level of 294 pmol/L on that medication. Patient #2 ceased that therapy two years prior to this study.

Other blood chemistry remained within normal range and there were no adverse events associated with the application including no abnormality or irritation associated with the skin at the application site. There was no discomfort associated with the application. Dermal photography revealed the commencement of natural skin shedding of the microparticles from 7 days after the application, but this was not visible without magnified photography.

TABLE 11
Serum estradiol levels in post-menopausal female patients after
topical application of estradiol-containing Foroderm compositions

| | | | Yrs | | | Baseline | | | | | |
|---|---|---|---|---|---|---|---|---|---|---|---|
| | | | post- | Skin | Skin | Serum | | | | | |
| | Age | | meno- | thickness | elasticity | estradiol | 4 | 24 | 72 | 168 | 40 |
| Patient | (yrs) | BMI | pause | (mm) | (kPa) | (pmol/L) | hrs | hrs | hrs | hrs | days |
| Average | 54 | 28 | 0 | 0.9-3.3 | 13-32 | 5-112 | | | | | |
| Meno- | | | | | | | | | | | |
| pause | | | | | | | | | | | |
| 1 | 59.6 | 22.6 | 10 | 0.8 | 13 | <88 | 93 | <88 | <88 | <88 | <88 |
| 2 | 70.5 | 37 | 18 | 0.7 | 12 | <88 | 91 | 94 | <88 | 108 | <88 |
| 3 | 76.5 | 25.5 | 25 | 0.5 | 9 | <88 | <88 | 103 | <88 | 119 | <88 |
| Mean | 68.5 | 27.7 | 16.5 | 0.65 | 11.2 | <88 | NA | NA | NA | NA | NA |

NA = Not Applicable.

### Summary

This pilot study in women with very thin and fragile skin demonstrated that a clinically significant dosage of estradiol could be delivered into the dermis of the face in women with extremely low skin thickness without systemic absorption of the hormone. The application was painless and there were no adverse skin reactions either early or later in the trial with no delayed Depo secretion of estradiol at 40 days.

### Example 11—Optimisation of Foroderm Application

These experiments were conducted to determine an enhanced application procedure for the microparticles of the compositions disclosed herein.

### Experiment

Micropatterned applicators were fabricated using micromilling at UniSA-ANFF to apply Foroderm. The micropatterned applicators were made using ridge spacing from 20-200 μm. The interaction between different solvent and an applicator head was also tested. These applicators were used to apply the Foroderm particles (Fiberglass) to excised human skin using our standard application parameters, 30 seconds application, weight on the applicator during application and 5 mg Foroderm. The skin was then imaged and microparticles were counted per mm². Transepidermal water loss (TEWL) was used to determine the level of barrier disruption.

### Materials and Methods

### Profiling Parameters

The solvents that were tested were ethanol, DMSO (dimethyl sulfoxide), Tween-80 and UDA (undecanoic acid). The applicators were profiled by the Olympus OLS5000 laser confocal microscope. The settings for the microscope were LM 20× lens, 3D fine plus colour scanning mode and 1× zoom. Selected roughness profiles were measured individually over peaks and troughs (Ra). A 500 μm wide roughness profile (Ra) and area roughness of total scanned area (Sa) was also measured.

### Applicator Applications in Ex Vivo Human Skin

These applicators were applied to the ex vivo human skin with 50 μl of Rhodamine 6G for visualisation (0.02%) in 70% ethanol plus 5 mg of Foroderm. The treated skin was incubated for 60 minutes and the excess of particles and solution was gently removed by cotton buds. TEWL was measured straight away. Then the treated skin was tap-stripped 5 times to remove stratum corneum (skin surface) before being imaged under a confocal microscope.

### Results

### Roughness Comparison

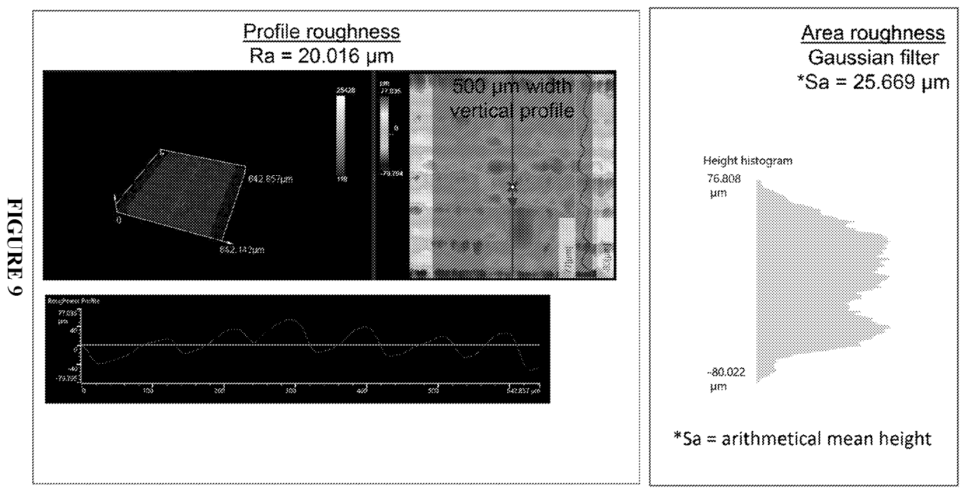

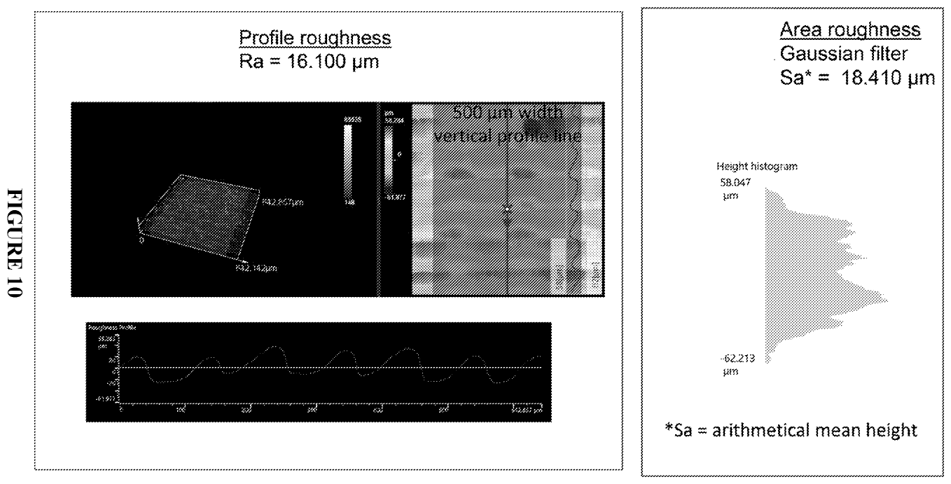

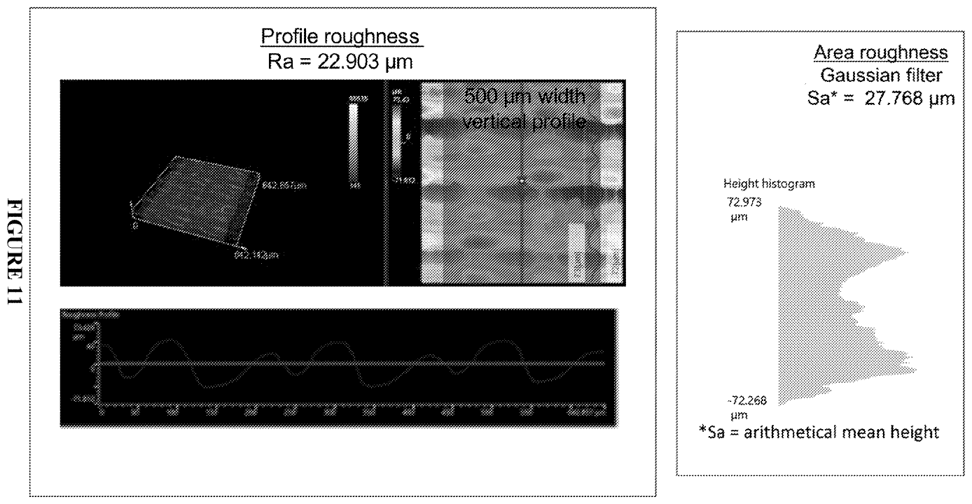

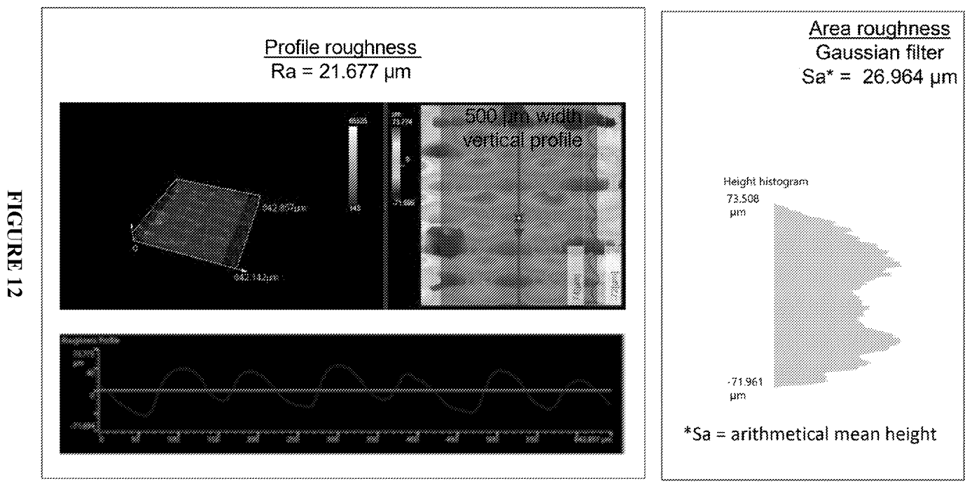

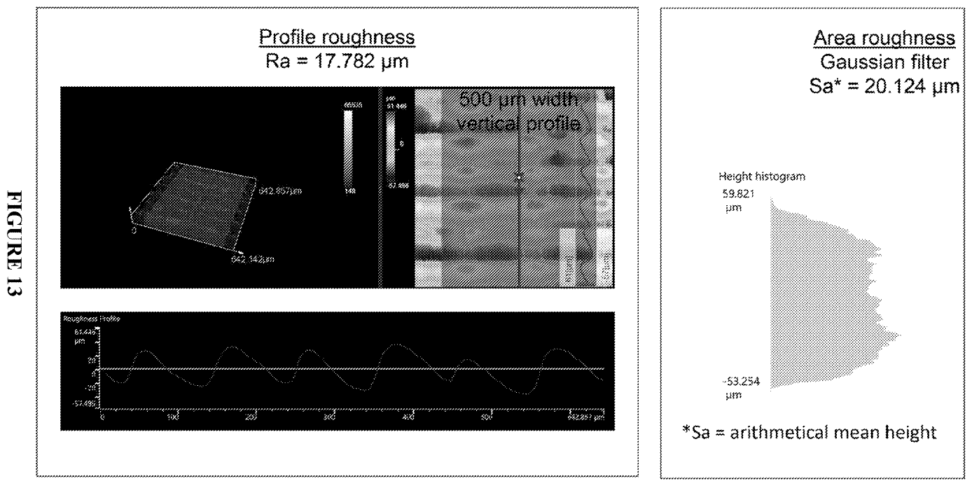

FIGS. 9-13 show the applicator profiles including roughness and height histogram. FIG. 9 shows the default profile for an applicator head (scanned area). When the applicator head was interacted with ethanol and UDA, the head roughness was decreased slightly (FIGS. 10 and 13) however, with DMSO and Tween-80, the head roughness was increased slightly (FIGS. 11 and 12). Table 12 shows the summary of roughness in each solvent in quantitative manner.

TABLE 12
Summary of roughness comparison

| Applicator | Roughness | | | | | |
|---|---|---|---|---|---|---|
| Type | (μm) | NT | Eth | DMSO | T80 | UDA |
| Pen | Surface (Sa) | 25.669 | 18.410 | 27.768 | 26.964 | 20.124 |
| | Profile, 500 μm | 20.016 | 16.100 | 22.903 | 21.677 | 17.782 |
| | (Ra) | | | | | |

### Transepidermal Water Loss (TEWL) Measurement

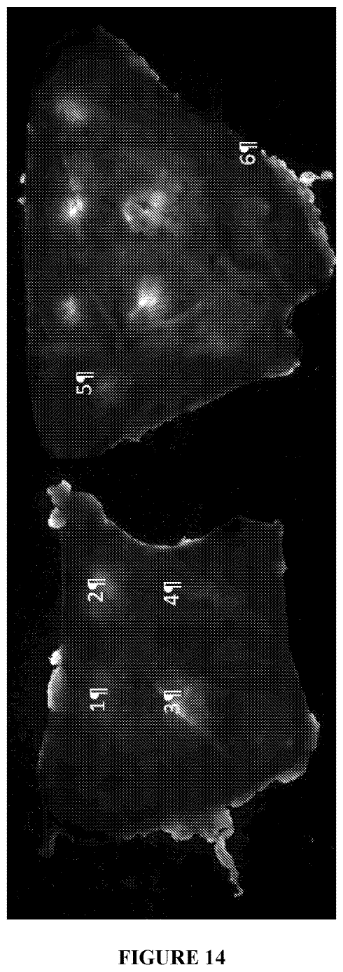

These applicators were used to apply Foroderm with Rhodamine 6G (Rh6G) (for visualisation). 5 mg of Foroderm was massaged into the skin with an applicator for 30 seconds with 800 g of application force. TEWL was measured immediately after the application (Table 13). The area 2, 3 and 5 showed the disruptive surface skin with Foroderm plus applicator, which resulted in delivering Rh6G below the surface of skin (FIG. 14).

TABLE 13
Transdermal Water Loss (TEWL) in treated area.

| | | | Std. | | | Int. | Raw Int. | |
|---|---|---|---|---|---|---|---|---|
| | Area | Mean | Dev. | Min | Max | Density | Density | Mean± |
| 1 | 2025 | 26.013 | 8.663 | 11 | 58 | 52677 | 52677 | 34.073 ± |
| | | | | | | | | 11.886 |
| 2 | 2025 | 35.818 | 21.993 | 8 | 106 | 72531 | 72531 | |
| 3 | 2025 | 50.190 | 30.949 | 9 | 200 | 101634 | 101634 | |
| 4 | 2025 | 24.271 | 12.953 | 7 | 84 | 49148 | 49148 | |
| 5 | 2025 | 19.564 | 5.732 | 8 | 38 | 39618 | 39618 | 22.231 ± |
| | | | | | | | | 3.772 |
| 6 | 2025 | 24.898 | 9.822 | 7 | 79 | 50419 | 50419 | |

Area 1 = Rh 6G only, Area 2 = Foroderm + applicator + Rh6G, Area 3 = Foroderm + applicator + Rh6G, Area 4 = Rh 6G only, Area 5 = Foroderm + applicator + Rh6G, Area 6 = Rh 6G only.

### Outcomes

Quantitative data was generated to show the applicator head profiles and effects of surface roughness by interacting with few commonly used transdermal chemical penetration enhancers. Based on TEWL values and delivery profile, the defined protocol for use of 3D printed applicator was to use 5 mg of Foroderm with applicator for 30 seconds by using 800 g of weight on the applicator during application.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A composition comprising:
a plurality of microparticles;
at least one bioactive compound, wherein the at least one bioactive compound is optionally a coating on at least a portion of the plurality of microparticles; and
a penetration enhancer,
wherein the composition is formulated as a topical composition.

2. A composition comprising:
a plurality of microparticles;
at least one bioactive compound, wherein the at least one bioactive compound is optionally a coating on at least a portion of the plurality of microparticles; and
a penetration enhancer,
wherein the composition is formulated as a topical composition, and with the proviso that no bioactive compound, or substantially no bioactive compound, is delivered to the bloodstream following application of the composition to a portion of skin on a subject.

3. A composition that is applied topically, comprising: a plurality of microparticles; at least one bioactive compound that is a ligand of the nuclear receptor superfamily; and a penetration enhancer.

4. The composition according to any one of the preceding claims, wherein at least a portion, substantially all, or all of the microparticles are elongate.

5. The composition according to any one of the preceding claims, wherein the composition is applied to the stratum corneum of the skin.

6. The composition according to any one of the preceding claims, wherein the at least one bioactive compound is selected from the group consisting of: a therapeutic compound, a cosmeceutical, a pharmaceutical, a nutraceutical, a diagnostic agent, and mixtures thereof.

7. The composition according to any one of the preceding claims, wherein the at least one bioactive compound is: a steroid or a corticosteroid, optionally selected from the group consisting of: estradiol, estrone, estriol, 5α-dihydrotestosterone, finasteride, cortisone, spironolactone, or a mixture thereof, minoxidil, minoxidil sulphate, or a derivative or a mixture thereof, a vitamin, optionally selected from vitamin A, B, C, D3, and/or E; or a retinoid, optionally selected from the group consisting of: retinoic acid, tretinoin; and mixtures thereof.

8. The composition according to any one of the preceding claims, wherein the composition comprises a penetration enhancer selected from the group selected from: non-polar solvents, polar protic solvents, aprotic polar solvents, alcohols, polyols, ionic surfactants, non-ionic surfactants solvents, aliphatic hydrocarbons, fatty acids, and mixtures thereof, wherein the penetration enhancer is optionally selected from the group consisting of: ethanol, dimethyl sulfoxide, propylene glycol, isopropanol, polyoxyethylene (20) sorbitan monooleate, urea, sodium docecyl sulfate, menthol, limonene, oleic acid, isopropyl myristate, undecanoic acid, and mixtures thereof.

9. The composition according to any one of the preceding claims, wherein the microparticles comprise or consist of: silicon, silica, and mixtures thereof.

10. The composition according to any one of the preceding claims, wherein the microparticles are coated with a material, wherein the material is optionally a dry coating and is optionally selected from the group consisting of: alginate, cross-linked alginate, carboxymethyl cellulose, and mixtures thereof.

11. The composition according to any one of the preceding claims, wherein the microparticles possess at least one of the following characteristics:
the microparticles are cylindrical, or substantially cylindrical;
the microparticles are elongate, optionally with an aspect ratio of from about 5:1 to about 50:1, optionally from about 10:1 to about 20:1;
the microparticles are etched;
the microparticles are hollow, solid or a mixture thereof,
at least a portion of the microparticles have one or more substantially flat end;
at least a portion of the microparticles have one or more substantially convex ends;
the strength of the microparticles is such as to withstand applied forces of at least 0.1 Newtons; and/or
the microparticles are substantially uniform in size.

12. The composition according to any one of the preceding claims, wherein the composition is in the form of a liquid, gel, ointment, cream, lotion, paste, salve, spray, or any mixture thereof.

13. The composition according to any one of the preceding claims, wherein the composition further comprises:
at least one solvent, optionally selected from the group consisting of: water, ethanol, and mixtures thereof, and/or
gelling and/or crosslinking agent, optionally selected from the group consisting of: carboxymethylcellulose, a salt, and mixtures thereof, and wherein the salt of optionally selected from the group consisting of: calcium chloride, and mixtures thereof.

14. The composition according to any one of the preceding claims with the proviso that no bioactive compound, or substantially no bioactive compound, is delivered to the bloodstream following application of the composition to a portion of skin on a subject.

15. A pharmaceutical composition comprising the composition according to any one of claims 1 to 14, and at least one pharmaceutically acceptable excipient.

16. A cosmeceutical composition comprising the composition according to any one of claims 1 to 14, and at least one excipient, optionally at least one pharmaceutically acceptable excipient.

17. An applicator that administers a composition according to any one of claims 1 to 14, a pharmaceutical composition according to claim 15, or a cosmeceutical composition according to claim 16.

18. The applicator according to claim 17, wherein the applicator comprises a microtextured surface.

19. The applicator according to claim 18, wherein the microtextured surface includes angled faces that define spaces to receive the microparticles during use of the applicator.

20. A method of treating and/or preventing at least one menopause symptom in a subject, the method comprising administering a composition according to any one of claims 1 to 14, a pharmaceutical composition according to claim 15, or a cosmeceutical composition according to claim 16, to the subject.

21. Use of a composition according to any one of claims 1 to 14, a pharmaceutical composition according to claim 15, or a cosmeceutical composition according to claim 16, in the manufacture of a medicament for the treatment and/or prevention of at least one menopause symptom in a subject.

22. A composition according to any one of claims 1 to 14, a pharmaceutical composition according to claim 15, or a cosmeceutical composition according to claim 16, when used in the treatment and/or prevention of at least one menopause symptom in a subject.

23. The method according to claim 20, the use according to claim 21, or the composition when used according to claim 22, wherein the at least one menopause symptom is selected from the group consisting of: hot flushes, night sweats, crawling or itching sensations under the skin, headache, tiredness, urinary frequency, vaginal dryness, and mixtures thereof.

24. A method of hydrating and/or increasing collagen formation in at least a portion of skin on a subject, the method comprising administering a composition according to any one of claims 1 to 14, a pharmaceutical composition according to claim 15, or a cosmeceutical composition according to claim 16, to the subject.

25. Use of a composition according to any one of claims 1 to 14, a pharmaceutical composition according to claim 15, or a cosmeceutical composition according to claim 16, to hydrate and/or increase collagen formation in at least a portion of skin on a subject.

26. A composition according to any one of claims 1 to 14, a pharmaceutical composition according to claim 15, or a cosmeceutical composition according to claim 16, when used to hydrate and/or increase collagen formation in at least a portion of skin on a subject.

27. A method of improving or treating the appearance of skin on a subject comprising administering to the skin an effective amount of a composition according to any one of claims 1 to 14, a pharmaceutical composition according to claim 15, or a cosmeceutical composition according to claim 16, to the subject.

28. Use of a composition according to any one of claims 1 to 14, a pharmaceutical composition according to claim 15, or a cosmeceutical composition according to claim 16, to improve or treat the appearance of skin on a subject.

29. A composition according to any one of claims 1 to 14, a pharmaceutical composition according to claim 15, or a cosmeceutical composition according to claim 16, when used to improve or treat the appearance of skin on a subject.

30. A method of treating and/or preventing a hair loss on a subject, the method comprising administering a composition according to any one of claims 1 to 14, a pharmaceutical composition according to claim 15, or a cosmeceutical composition according to claim 16 to the subject, wherein the composition is a topical composition, and wherein the at least one bioactive compound optionally comprises minoxidil or a derivative thereof, minoxidil sulphate or a derivative thereof, finasteride or a derivative thereof, or a mixture thereof.

31. Use of a composition according to any one of claims 1 to 14, a pharmaceutical composition according to claim 15, or a cosmeceutical composition according to claim 16, in the manufacture of a medicament to treat and/or prevent hair loss on a subject, wherein the composition is a topical composition, and wherein the at least one bioactive compound optionally comprises minoxidil or a derivative thereof, minoxidil sulphate or a derivative thereof, finasteride or a derivative thereof, or a mixture thereof.

32. A composition according to any one of claims 1 to 14, a pharmaceutical composition according to claim 15, or a cosmeceutical composition according to claim 16, when used to treat and/or prevent a subject, wherein the composition is a topical composition, and wherein the at least one bioactive compound optionally comprises minoxidil or a derivative thereof, minoxidil sulphate or a derivative thereof, finasteride or a derivative thereof, or a mixture thereof.

33. The method according to claim 30, the use according to claim 31, or the composition when used according to claim 32, wherein the at least one bioactive compound comprises minoxidil or a derivative thereof, minoxidil sulphate or a derivative thereof, or a mixture thereof.

34. The method or use according to any one of claims 30 to 33, wherein the hair loss is male balding.

35. A method of treating and/or preventing a hirsutism in a subject, the method comprising administering a composition according to any one of claims 1 to 14, a pharmaceutical composition according to claim 15, or a cosmeceutical composition according to claim 16, to the subject, wherein the composition is a topical composition, and wherein the at least one bioactive compound optionally comprises spironolactone or a derivative thereof.

36. Use of a composition according to any one of claims 1 to 14, a pharmaceutical composition according to claim 15, or a cosmeceutical composition according to claim 16, in the manufacture of a medicament for treating and/or preventing a hirsutism in a subject, wherein the composition is a topical composition, and wherein the at least one bioactive compound optionally comprises spironolactone or a derivative thereof.

37. A composition according to any one of claims 1 to 14, a pharmaceutical composition according to claim 15, or a cosmeceutical composition according to claim 16, when used to treat and/or prevent hirsutism in a subject, wherein the composition is a topical composition, and wherein the at least one bioactive compound optionally comprises spironolactone or a derivative thereof.

38. The method according to claim 35, the use according to claim 36, or the composition when used according to claim 37, wherein the at least one bioactive compound comprises spironolactone or a derivative thereof.

39. A method of treating and/or preventing a dermatological condition or disease in a subject, the method comprising administering a composition according to any one of claims 1 to 14, a pharmaceutical composition according to claim 15, or a cosmeceutical composition according to claim 16, to the subject.

40. Use of a composition according to any one of claims 1 to 14, a pharmaceutical composition according to claim 15, or a cosmeceutical composition according to claim 16, in the manufacture of a medicament for treating and/or preventing a dermatological condition or disease in a subject.

41. A composition according to any one of claims 1 to 14, a pharmaceutical composition according to claim 15, or a cosmeceutical composition according to claim 16, when used to treat and/or prevent a dermatological condition or disease in a subject.

42. The method according to claim 39, the use according to claim 40, or the composition when used according to claim 41, wherein the dermatological condition or disease is acne.

43. A method of delivering at least one bioactive compound through the stratum corneum of skin on a subject for cosmeceutical purposes, excluding skin malignancies, the method comprising administering a composition according to any one of claims 1 to 14, a pharmaceutical composition according to claim 15, or a cosmeceutical composition according to claim 16, wherein the bioactive compound is optionally estradiol, estrone, estriol or a retinoid, optionally in the presence of a further hormonal modulator.

44. Use of a composition according to any one of claims 1 to 14, a pharmaceutical composition according to claim 15, or a cosmeceutical composition according to claim 16, to deliver at least one bioactive compound through the stratum corneum of skin on a subject for cosmeceutical purposes, excluding skin malignancies, wherein the bioactive compound is optionally estradiol, estrone, estriol or a retinoid, optionally in the presence of a further hormonal modulator.

45. A composition according to any one of claims 1 to 14, a pharmaceutical composition according to claim 15, or a cosmeceutical composition according to claim 16, when used to deliver at least one bioactive compound through the stratum corneum of skin on a subject for cosmeceutical purposes, excluding skin malignancies, wherein the bioactive compound is optionally estradiol, estrone, estriol or a retinoid, optionally in the presence of a further hormonal modulator.

46. The method or use according to any one of claims 20 to 45, wherein a composition according to any one of claims 1 to 14, a pharmaceutical composition according to claim 15, or a cosmeceutical composition according to claim 16, is administered at least once daily to a subject, for a period of at least 1 month.

47. The method or use according to any one of claims 20 to 46, wherein a composition according to any one of claims 1 to 14, a pharmaceutical composition according to claim 15, or a cosmeceutical composition according to claim 16, is administered with the aid of an applicator according to any one of claims 17 to 19.

48. The method or use according to any one of claims 20 to 47, wherein the subject is a human.
